(19) United States
(12) Patent Application Publication
(10) Pub. No.: US 2026/0166169 A1
(43) Pub. Date: Jun. 18, 2026

(54) ANTIBODY-DRUG CONJUGATE, PREPARATION METHOD AND USE THEREOF

(71) Applicant: MULTITUDE THERAPEUTICS INC., Shanghai (CN)

(72) Inventors: Xun MENG, Shanghai (CN); Shu-Hui LIU, Shanghai (CN); Jing SHI, Shanghai (CN); Weining WENG, Shanghai (CN)

(73) Assignee: MULTITUDE THERAPEUTICS INC., Shanghai (CN)

(21) Appl. No.: 19/119,037

(22) PCT Filed: Oct. 18, 2023
(86) PCT No.: PCT/CN2023/125253

§ 371 Date: Apr. 7, 2025

(51) Int. Cl. A61K 47/68; A61P 35/00; C07K 16/28; C07K 16/30

(52) U.S. Cl. CPC A61K 47/68037; A61K 47/6849; A61K 47/6851; A61K 47/6889; A61P 35/00; C07K 16/2884; C07K 16/3092

(57) ABSTRACT

An antibody-drug conjugate, preparation method and use thereof are provided. The antibody-drug conjugate includes an antibody, a cytotoxic payload and a linker of formula I, wherein a succinimidyl group of the linker of formula I forms a thioether bond with a thiol group obtained by a reduction of an interchain disulfide chain of the antibody, a carbonyl group in an ester group of the linker is connected to an amino group of the payload. The antibody-drug conjugate has use in the manufacture of a therapeutic agent for diagnosis, prevention and treatment of neoplastic disease.

### CROSS-REFERENCE TO RELATED APPLICATIONS

This application is the national phase entry of International Application No. PCT/CN2023/125253, filed on Oct. 18, 2023, which is based upon and claim priority to PCT International Application No. PCT/CN2022/126273, filed on Oct. 19, 2022, and PCT International Application No. PCT/CN2023/118363, filed on Sep. 12, 2023, the entire contents of which are incorporated herein by reference.

### SEQUENCE LISTING STATEMENT

A sequence listing in compliance with WIPO Standard ST.26 is submitted herewith as a separate electronic file entitled “DCF221353WO.xml,” created on 2023 Sep. 28, and having a size of 93.1 kilobytes. The sequence listing contained in the XML file complies with the requirements of WIPO Standard ST.26 and forms part of the disclosure of this application. The content of the sequence listing is incorporated by reference in its entirety.

### TECHNICAL FIELD

The present disclosure relates to an antibody-drug conjugate that links an antibody to a drug or toxin with antitumor activity by a linker and plays its anti-tumor role against tumor cells.

### BACKGROUND

The statements in this section merely provide background information related to the present disclosure and do not necessarily constitute prior art.

Antibody-drug conjugate (ADC) is a vectorized chemotherapy, and selectively deliver the cytotoxic drugs in the tumor/cancer cell (Antibody-drug conjugates: The Last Decade, Nicolas Joubert, et al., Pharmaceuticals (Basel). 2020 Sep. 14; 13 (9): 245). With the development of drug delivery technology, ADC targeted delivery technology can effectively overcome the side effects caused by poor water solubility and insufficient tissue distribution of camptothecins. The marketed ADC drugs Enhertu and Sacituzumab govitecan have superior effects in the treatment of tumors, especially malignant tumors. Both of Enhertu and Sacituzumab govitecan use DNA topoisomerase inhibitor of camptothecin derivatives that are more hydrophobic than tubulin inhibitors (e.g., MMAE and MMAF) as cytotoxic drugs. Sacituzumab govitecan uses MCC-triazole spacer-PEG7-lysine-PABC as a linker to decompose and release camptothecin SN38 in cell lysosomes (U.S. Ser. No. 13/948,732). Enhertu developed by AstraZeneca/Daiichi sankyo uses cathepsin B-activated GGFG (an amino acid sequence composed of glycine-glycine-phenylalanine-glycine linked by peptide bonds) tetrapeptide as a linker and introduces self-cleavage structure to release Exatecan derivative Dxd (Yusuke Ogitani et al., Clin Cancer Res (2016) 22 (20): 5097-5108). However, the above cytotoxic drugs MMAE, SN38, and Dxd are all substrates of P-glycoprotein (P-gp) (Front Pharmacol 2019; 10:749), and may have drug resistance to some tumors with high expression of P-gp.

Therefore, there remains a need for new antibody-drug conjugates for delivering cytotoxic drugs within tumor/cancer cells.

### SUMMARY

The present disclosure provides an antibody-drug conjugate, or an isomer, an isotopic variant, a pharmaceutically acceptable salt, prodrug, solvate thereof, or combinations thereof, which includes an anti-MUC18 or anti-CD44v7/8 antibody or antigen-binding fragment thereof, a payload and a linker of formula I,

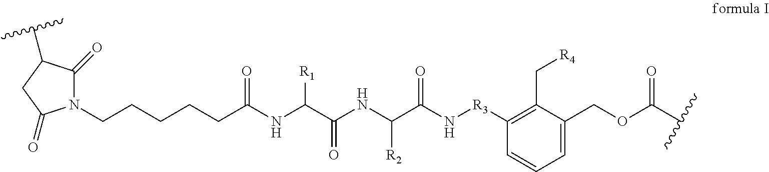

A succinimidyl group of the linker of formula I forms a thioether bond with a thiol group obtained by a reduction of an interchain disulfide chain of the antibody or antigen-binding fragment thereof;
A carbonyl group in an ester group of the linker of formula I is connected to an amino group of the payload;
R₁ and R₂ independently are hydrogen, methyl or isopropyl group;
R₃ represents —(CR₅HCONH)n¹-(CH₂CONH)n²- or a single bond, R₅ is hydrogen or benzyl, n¹ represents an integer of 0 to 2, and n² represents an integer of 0 to 2;
R₄ represents a methylamino group or —(NCH₃COCH₂)n³-NCH₃COCH₃, and n³ represents an integer of 1 to 20.

In some embodiments, R₄ represents —(NCH₃COCH₂)n³-NCH₃COCH₃, and n³ represents an integer of 8 to 15.

In some embodiments, R₄ represents —(NCH₃COCH₂)n³-NCH₃COCH₃, n³ represents an integer of 10 to 12.

In some embodiments, R₃ represents —(CR₅HCONH)n¹-(CH₂CONH)n²- or a single bond, R₅ is benzyl, n¹ represents 1 or 2, and n² represents 1 or 2, R₄ represents —(NCH₃COCH₂)n³-NCH₃COCH₃, n³ represents an integer of 8 to 15.

In some embodiments, R₃ represents a single bond, R₄ represents —(NCH₃COCH₂)n³-NCH₃COCH₃, and n³ represents an integer of 8 to 15.

In some embodiments, R₃ represents a single bond, and R₄ represents a methylamino group.

In some embodiments, the linker is selected from any one of the following:

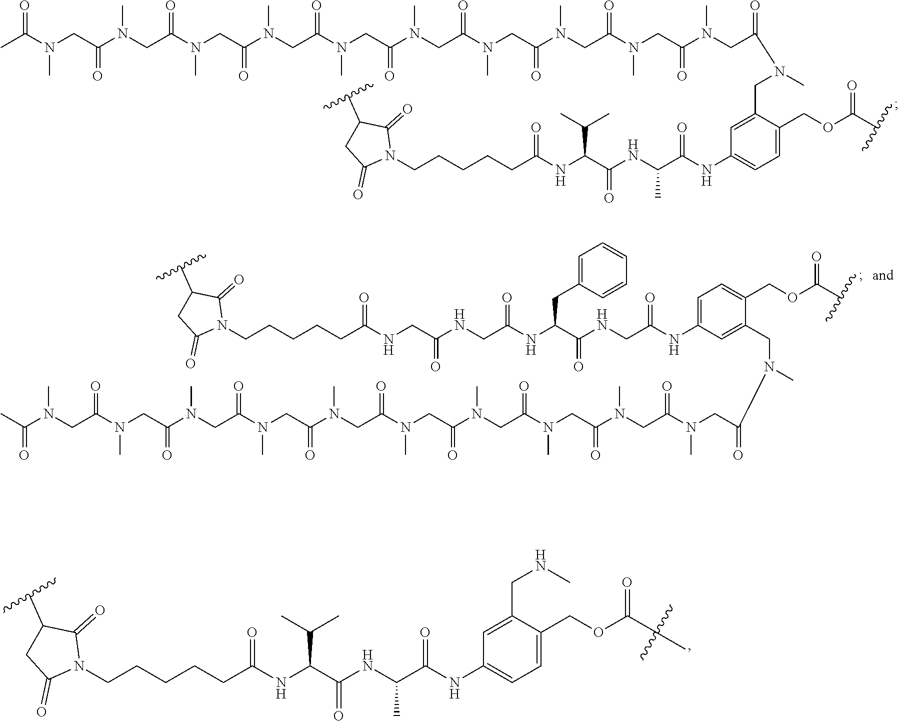 [SMILES: *C(=O)OCc1ccc(NC(=O)[C@H](C)NC(=O)[C@@H](NC(=O)CCCCCN2C(=O)CC(*)C2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *C(=O)OCc1ccc(NC(=O)CNC(=O)[C@H](Cc2ccccc2)NC(=O)CNC(=O)CNC(=O)CCCCCN2C(=O)CC(*)C2=O)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *C(=O)OCc1ccc(NC(=O)[C@H](C)NC(=O)[C@@H](NC(=O)CCCCCN2C(=O)CC(*)C2=O)C(C)C)cc1CNC]

or combinations thereof.

In some embodiments, the payload is at least one selected from the group consisting of cytotoxic agents, labels, nucleic acids, radionuclides, hormones, immunomodulators, prodrug converting enzymes, ribonucleases, agonistic antibodies, antagonistic antibodies and fragments thereof, fusion proteins or derivatives thereof.

In some embodiments, the cytotoxic agent includes a tubulin inhibitor and/or a topoisomerase inhibitor, the tubulin inhibitor includes auristatin or derivatives thereof, maytansine or derivatives thereof, the topoisomerase inhibitor includes camptothecin and derivatives thereof.

In some embodiments, the payload is exatecan of formula II, which is connected to the linker by a nitrogen atom of an amino group on a cyclohexane ring thereof,

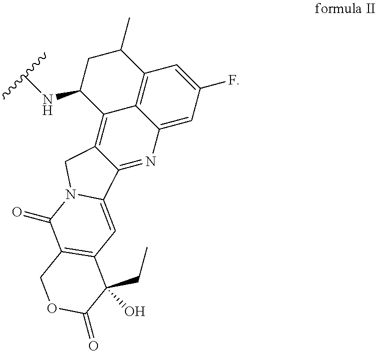 [SMILES: *N[C@H]1CC(C)c2cc(F)cc3nc4c(c1c23)Cn1c-4cc2c(c1=O)COC(=O)[C@]2(O)CC]

In some embodiments, the anti-MUC18 antibody or antigen-binding fragment thereof includes any one of the following:
HCDR1 with amino acid sequence as shown in SEQ ID NO: 4, HCDR2 with amino acid sequence as shown in SEQ ID NO: 16, HCDR3 with amino acid sequence as shown in SEQ ID NO: 28, and LCDR1 with amino acid sequence as shown in SEQ ID NO: 38, LCDR2 with amino acid sequence STS, LCDR3 with amino acid sequence as shown in SEQ ID NO: 52;
HCDR1 with amino acid sequence as shown in SEQ ID NO: 1, HCDR2 with amino acid sequence as shown in SEQ ID NO: 11, HCDR3 with amino acid sequence as shown in SEQ ID NO: 23, and LCDR1 with amino acid sequence as shown in SEQ ID NO: 34, LCDR2 with amino acid sequence LAS, LCDR3 with amino acid sequence as shown in SEQ ID NO: 46;
HCDR1 with amino acid sequence as shown in SEQ ID NO: 1, HCDR2 with amino acid sequence as shown in SEQ ID NO: 12, HCDR3 with amino acid sequence as shown in SEQ ID NO: 23, and LCDR1 with amino acid sequence as shown in SEQ ID NO: 35, LCDR2 with amino acid sequence LAS, LCDR3 with amino acid sequence as shown in SEQ ID NO: 47;
HCDR1 with amino acid sequence as shown in SEQ ID NO: 2, HCDR2 with amino acid sequence as shown in SEQ ID NO: 13, HCDR3 with amino acid sequence as shown in SEQ ID NO: 24, and LCDR1 with amino acid sequence as shown in SEQ ID NO: 36, LCDR2 with amino acid sequence NAK, LCDR3 with amino acid sequence as shown in SEQ ID NO: 48;
HCDR1 with amino acid sequence as shown in SEQ ID NO: 3, HCDR2 with amino acid sequence as shown in SEQ ID NO: 14, HCDR3 with amino acid sequence as shown in SEQ ID NO: 25, and LCDR1 with amino acid sequence as shown in SEQ ID NO: 37, LCDR2 with amino acid sequence FAS, LCDR3 with amino acid sequence as shown in SEQ ID NO: 49;
HCDR1 with amino acid sequence as shown in SEQ ID NO: 4, HCDR2 with amino acid sequence as shown in SEQ ID NO: 15, HCDR3 with amino acid sequence as shown in SEQ ID NO: 26, and LCDR1 with amino acid sequence as shown in SEQ ID NO: 38, LCDR2 with amino acid sequence STS, LCDR3 with amino acid sequence as shown in SEQ ID NO: 50;
HCDR1 with amino acid sequence as shown in SEQ ID NO: 4, HCDR2 with amino acid sequence as shown in SEQ ID NO: 15, HCDR3 with amino acid sequence as shown in SEQ ID NO: 27, and LCDR1 with amino acid sequence as shown in SEQ ID NO: 39, LCDR2 with amino acid sequence STS, LCDR3 with amino acid sequence as shown in SEQ ID NO: 51;
HCDR1 with amino acid sequence as shown in SEQ ID NO: 5, HCDR2 with amino acid sequence as shown in SEQ ID NO: 17, HCDR3 with amino acid sequence as shown in SEQ ID NO: 29, and LCDR1 with amino acid sequence as shown in SEQ ID NO: 40, LCDR2 with amino acid sequence WAS, LCDR3 with amino acid sequence as shown in SEQ ID NO: 53;
HCDR1 with amino acid sequence as shown in SEQ ID NO: 6, HCDR2 with amino acid sequence as shown in SEQ ID NO: 18, HCDR3 with amino acid sequence as shown in SEQ ID NO: 29, and LCDR1 with amino acid sequence as shown in SEQ ID NO: 41, LCDR2 with amino acid sequence WAS, LCDR3 with amino acid sequence as shown in SEQ ID NO: 53;
HCDR1 with amino acid sequence as shown in SEQ ID NO: 7, HCDR2 with amino acid sequence as shown in SEQ ID NO: 19, HCDR3 with amino acid sequence as shown in SEQ ID NO: 30, and LCDR1 with amino acid sequence as shown in SEQ ID NO: 42, LCDR2 with amino acid sequence RTS, LCDR3 with amino acid sequence as shown in SEQ ID NO: 54;
HCDR1 with amino acid sequence as shown in SEQ ID NO: 8, HCDR2 with amino acid sequence as shown in SEQ ID NO: 20, HCDR3 with amino acid sequence as shown in SEQ ID NO: 31, and LCDR1 with amino acid sequence as shown in SEQ ID NO: 43, LCDR2 with amino acid sequence WAS, LCDR3 with amino acid sequence as shown in SEQ ID NO: 55;
HCDR1 with amino acid sequence as shown in SEQ ID NO: 9, HCDR2 with amino acid sequence as shown in SEQ ID NO: 21, HCDR3 with amino acid sequence as shown in SEQ ID NO: 32, and LCDR1 with amino acid sequence as shown in SEQ ID NO: 44, LCDR2 with amino acid sequence WAS, LCDR3 with amino acid sequence as shown in SEQ ID NO: 56; and
HCDR1 with amino acid sequence as shown in SEQ ID NO: 10, HCDR2 with amino acid sequence as shown in SEQ ID NO: 22, HCDR3 with amino acid sequence as shown in SEQ ID NO: 33, and LCDR1 with amino acid sequence as shown in SEQ ID NO: 45, LCDR2 with amino acid sequence LMS, LCDR3 with amino acid sequence as shown in SEQ ID NO: 57.

In some embodiments, the anti-MUC18 antibody or antigen-binding fragment thereof includes:
heavy chain variable region with amino acid sequence as shown in SEQ ID NO: 84, and light chain variable region with amino acid sequence as shown in SEQ ID NO: 85;
heavy chain variable region with amino acid sequence as shown in SEQ ID NO: 86, and light chain variable region with amino acid sequence as shown in SEQ ID NO: 87; or
heavy chain variable region with amino acid sequence as shown in SEQ ID NO: 88, and light chain variable region with amino acid sequence as shown in SEQ ID NO: 89, or conservative variants thereof.

In some embodiments, the anti-CD44 v7/8 antibody or antigen-binding fragment specifically binds to a binding peptide in human CD44 v7/8, wherein the binding peptide includes an amino acid sequence as shown in SEQ ID NO: 90.

In some embodiments, the anti-CD44 v7/8 antibody or antigen-binding fragment does not bind to a binding peptide with an amino acid sequence as shown in SEQ ID NO: 91 and/or SEQ ID NO: 92 in human CD44 v7/8.

In some embodiments, the anti-CD44 v7/8 antibody or antigen-binding fragment includes HCDR1 with amino acid sequence as shown in SEQ ID NO: 93, HCDR2 with amino acid sequence as shown in SEQ ID NO: 94, HCDR3 with amino acid sequence as shown in SEQ ID NO: 95; and the LCDR1 with amino acid sequence as shown in SEQ ID NO: 96, LCDR2 with amino acid sequence RAN, LCDR3 with amino acid sequence as shown in SEQ ID NO: 97.

In some embodiments, the anti-CD44v7/8 antibody or antigen-binding fragment thereof includes:
heavy chain variable region with amino acid sequence as shown in SEQ ID NO: 100, and light chain variable region with amino acid sequence as shown in SEQ ID NO: 101; or
   heavy chain variable region with amino acid sequence as shown in SEQ ID NO: 100, and light chain variable region with amino acid sequence as shown in SEQ ID NO: 104, or conservative variants thereof.

In some embodiments, a DAR is 1 to 10. In some embodiments, a DAR is 4 to 10.

The present disclosure provides a preparation method of the above antibody-drug conjugate, or an isomer, an isotopic variant, a pharmaceutically acceptable salt, prodrug, solvate thereof, or combinations thereof, which includes the following steps: reducing an anti-MUC18 or anti-CD44v7/8 antibody or antigen-binding fragment thereof such that interchain disulfide bonds thereof are at least partially reduced, and then reacting with a carbon atom at position 3 of maleimide-N-yl of the linker of formula III,

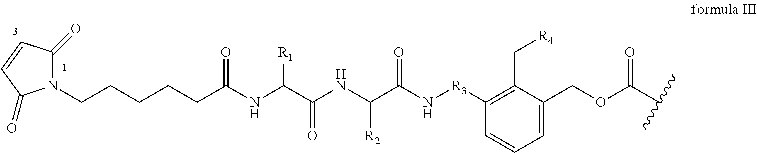

in the linker-payload, a carbonyl group in an ester group of the linker of formula III is connected to an amino group of the payload;
R₁ and R₂ independently are hydrogen, methyl or isopropyl group;
R₃ represents —(CR₅HCONH)n¹-(CH₂CONH)n²- or a single bond, R₅ is hydrogen or benzyl, n¹ represents an integer of 0 to 2, n² represents an integer of 0 to 2;
R₄ represents a methylamino group or —(NCH₃COCH₂)n³-NCH₃COCH₃, n³ represents an integer of 1 to 20.

In some embodiments, the method further includes: reacting the antibody or antigen-binding fragment thereof with a reducing agent in a buffer solution containing a chelating agent, and then adding a solution of the linker-payload, the linker having a structure of formula III, and adjusting pH of a reaction solution.

In some embodiments, the payload is exatecan of formula II, which is connected to a carbonyl group of an ester group in formula III by a nitrogen atom of an amino group on a cyclohexane ring thereof,

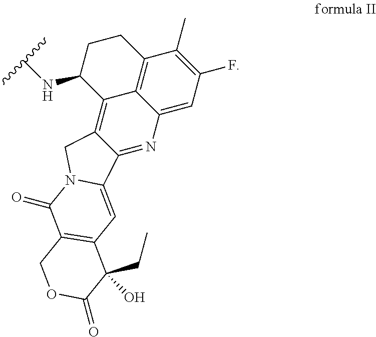 [SMILES: *N[C@H]1CCc2c(C)c(F)cc3nc4c(c1c23)Cn1c-4cc2c(c1=O)COC(=O)[C@]2(O)CC]

In some embodiments, a DAR is 1 to 10, optionally, 4 to 10.

The present disclosure provides a pharmaceutical composition, which includes the above antibody-drug conjugate, or an isomer, an isotopic variant, a pharmaceutically acceptable salt, prodrug, solvate thereof, or combinations thereof, and a pharmaceutically acceptable excipient.

The present disclosure provides a kit, which includes the antibody-drug conjugates described above, or an isomer, an isotopic variant, a pharmaceutically acceptable salt, prodrug, solvate thereof, or combinations thereof.

The present disclosure provides use of the above antibody-drug conjugate, the antibody-drug conjugate prepared by the above method, a pharmaceutical composition containing the above antibody-drug conjugate, or the kit in the manufacture of a therapeutic agent for diagnosis, prevention and treatment of neoplastic disease. In some embodiments, the tumor disease includes benign tumors and malignant tumors expressing MUC18 and/or CD44v7/8.

In some embodiments, the tumor disease includes melanoma, pharyngeal cancer, triple-negative breast cancer, esophageal adenocarcinoma, esophagus squamous cell carcinoma, liver cancer, ovarian cancer, cervical cancer, urothelial cancer, bladder neuroendocrine tumor, small cell lung cancer, non-small cell lung cancer, cutaneous squamous cell carcinoma, cholangiocarcinoma, metastatic pancreatic carcinoma, lung squamous cell carcinoma, head and neck squamous cell carcinoma and/or esophageal squamous cell carcinoma.

The present disclosure provides use of the above antibody-drug conjugate, the antibody-drug conjugate prepared by the above method, a pharmaceutical composition containing the above antibody-drug conjugate, or the kit in the manufacture of a therapeutic agent targeting MUC18 and/or CD44v7/8.

The present disclosure provides a method of diagnosing, preventing, and treating neoplastic diseases, which includes administering to a subject a therapeutically effective amount of a therapeutic agent, wherein the therapeutic agent includes the above antibody-drug conjugate, the antibody-drug conjugate prepared by the above method, or the above pharmaceutical composition.

### BRIEF DESCRIPTION OF THE DRAWINGS

FIG. 1A shows a graph of size exclusion chromatography of 02-1 naked antibody; FIG. 1B shows a graph of size exclusion chromatography of Hu1H2-2 naked antibody.

FIG. 2A shows a graph of hydrophobic interaction chromatography of 02-1 naked antibody;

FIG. 2B shows a graph of hydrophobic interaction chromatography of Hu1H2-2 naked antibody.

FIG. 3A shows a graph of size exclusion chromatography of antibody-drug conjugate 02-1-LP1 prepared in Example 5; FIG. 3B shows a detection graph of hydrophobic interaction chromatography of the antibody-drug conjugate 02-1-LP1.

FIG. 4A shows a graph of size exclusion chromatography of antibody-drug conjugate Hu1H2-2-LP1 prepared in Example 6; FIG. 4B shows a detection graph of hydrophobic interaction chromatography of the antibody-drug conjugate Hu1H2-2-LP1.

FIG. 5A shows a graph of size exclusion chromatography of antibody-drug conjugate Hu1H2-2-LP1 prepared in Example 7; FIG. 5B shows a detection graph of hydrophobic interaction chromatography of the antibody-drug conjugate Hu1H2-2-LP1.

FIG. 6A shows a graph of size exclusion chromatography of antibody-drug conjugate Hu1H2-2-LP2 prepared in Example 8; FIG. 6B shows a detection graph of hydrophobic interaction chromatography of the antibody-drug conjugate Hu1H2-2-LP2.

FIG. 7A shows a graph of size exclusion chromatography of antibody-drug conjugate Hu1H2-2-LP3 prepared in Example 9; FIG. 7B shows a detection graph of hydrophobic interaction chromatography of the antibody-drug conjugate Hu1H2-2-LP3.

FIG. 8A shows a size exclusion chromatography graph of antibody-drug conjugate 02-1-vc-MMAE prepared in Comparative Example 1; FIG. 8B shows a detection graph of hydrophobic interaction chromatography of the antibody-drug conjugate 02-1-vc-MMAE.

FIG. 9A shows a graph of size exclusion chromatography of antibody-drug conjugate Rituximab-vc-MMAE prepared in Comparative Example 2; FIG. 9B shows a detection graph of hydrophobic interaction chromatography of the antibody-drug conjugate Rituximab-vc-MMAE.

FIG. 10A shows a graph of size exclusion chromatography of antibody-drug conjugate Rituximab-LP1 prepared in Comparative Example 3; FIG. 10B shows a detection graph of hydrophobic interaction chromatography of the antibody-drug conjugate Rituximab-LP1.

FIG. 11A shows a graph of size exclusion chromatography of antibody-drug conjugate Human IgG-DXD prepared in Comparative Example 4; FIG. 11B shows a detection graph of hydrophobic interaction chromatography of the antibody-drug conjugate Human IgG-DXD.

FIG. 12A shows a graph of size exclusion chromatography of antibody-drug conjugate Human IgG-LP1 prepared in Comparative Example 5; FIG. 12B shows a detection graph of hydrophobic interaction chromatography of the antibody-drug conjugate Human IgG-LP1.

FIG. 13A shows a graph of size exclusion chromatography of antibody-drug conjugate Human IgG-LP1 prepared in Comparative Example 6; FIG. 13B shows a detection graph of hydrophobic interaction chromatography of the antibody-drug conjugate Human IgG-LP1.

FIG. 14A shows a graph of size exclusion chromatography of antibody-drug conjugate Human IgG-LP2 prepared in Comparative Example 7; FIG. 14B shows a detection graph of hydrophobic interaction chromatography of the antibody-drug conjugate Human IgG-LP2.

FIG. 15A shows a graph of size exclusion chromatography of antibody-drug conjugate Human IgG-LP3 prepared in Comparative Example 8; FIG. 15B shows a detection graph of hydrophobic interaction chromatography of the antibody-drug conjugate Human IgG-LP3.

FIG. 16A shows a graph of size exclusion chromatography of antibody-drug conjugate Hu1H2-2-DXD prepared in Comparative Example 9; FIG. 16B shows a detection graph of hydrophobic interaction chromatography of the antibody-drug conjugate Hu1H2-2-DXD.

FIG. 17 shows a time-fluorescence intensity statistics graph of endocytosis of A375 cells, HMVII cells, SK-MEL-2 cells and GAK cells to antibody-drug conjugate 02-1-LP1.

FIG. 18A1 shows a tumor volume-time changing curve of melanoma model mice treated with antibody-drug conjugates Rituximab-vc-MMAE, 02-1-vc-MMAE, Rituximab-LP1, and 02-1-LP1;

FIG. 18A2 is a partial view of FIG. 18A1, showing a tumor volume-time changing curve of 02-1-LP1, 02-1-vc-MMAE, and Vehicle groups.

FIG. 18B1 shows a weight-time changing curve of melanoma model mice treated with antibody-drug conjugates Rituximab-vc-MMAE, 02-1-vc-MMAE, Rituximab-LP1, and 02-1-LP1;

FIG. 18B2 is a partial view of FIG. 18B1, showing a tumor weight-time changing curve of 02-1-LP1, 02-1-vc-MMAE, and Vehicle groups.

FIG. 19 shows in vitro killing curves of antibody-drug conjugates against head and neck squamous cell carcinoma cell line Detroit562; FIG. 19A1 is a partial view of FIG. 19, showing in vitro killing curves of Hu1H2-2-LP2, Hu1H2-2-LP1, and Hu1H2-2-DXD; FIG. 19A2 is a partial view of FIG. 19, showing in vitro killing curves of Human IgG-LP2, Human IgG-LP1, and Human IgG-DXD.

FIG. 20 shows tumor volume-time changing curves of head and neck squamous cell carcinoma model mice treated with antibody-drug conjugates Hu1H2-2-LP1, Hu1H2-2-LP2, and Human IgG-LP1.

FIG. 21 shows weight-time changing curves of head and neck squamous cell carcinoma model mice treated with antibody-drug conjugates Hu1H2-2-LP1, Hu1H2-2-LP2, and Human IgG-LP1.

FIG. 22 shows tumor volume-time changing curves of lung cancer model mice treated with antibody-drug conjugates Hu1H2-2-LP1, Hu1H2-2-LP1 (DAR4), Hu1H2-2-LP3, Human IgG-LP1, Human IgG-LP1 (DAR4), and Human IgG-LP3.

FIG. 23 shows weight-time curves of lung cancer model mice treated with antibody-drug conjugates Hu1H2-2-LP1, Hu1H2-2-LP1 (DAR4), Hu1H2-2-LP3, Human IgG-LP1, Human IgG-LP1 (DAR4), and Human IgG-LP3.

FIG. 24A shows tumor volume-time changing curves of SCC-9 human head and neck squamous cell carcinoma CDX model treated with antibody-drug conjugates Rituximab-LP1 and 02-1-LP1. FIG. 24B shows weight-time curves of SCC-9 human head and neck squamous cell carcinoma CDX model treated with antibody-drug conjugates Rituximab-LP1 and 02-1-LP1.

FIG. 25A shows tumor volume-time changing curves of Huh-7 human liver cancer CDX model treated with antibody-drug conjugates Human IgG-LP1 and 02-1-LP1. FIG. 25B shows weight-time curves of Huh-7 human liver cancer CDX model treated with antibody-drug conjugates Human IgG-LP1 and 02-1-LP1.

FIG. 26A shows tumor volume-time changing curves of LD1-0015-200617 human esophagus squamous cell carcinoma PDX model treated with antibody-drug conjugates Human IgG-LP1 and 02-1-LP1. FIG. 26B shows weight-time curves of LD1-0015-200617 human esophagus squamous cell carcinoma PDX model treated with antibody-drug conjugates Human IgG-LP1 and 02-1-LP1.

FIG. 27A shows tumor volume-time changing curves of LD1-0016-390730 human esophagus adenocarcinoma PDX model treated with antibody-drug conjugate 02-1-LP1. FIG. 27B shows weight-time curves of LD1-0016-390730 human esophagus adenocarcinoma PDX model treated with antibody-drug conjugate 02-1-LP1.

FIG. 28A shows tumor volume-time changing curves of LD1-2025-362797 human small cell lung cancer PDX model treated with antibody-drug conjugate 02-1-LP1. FIG. 28B shows weight-time curves of LD1-2025-362797 human small cell lung cancer PDX model treated with antibody-drug conjugate 02-1-LP1.

FIG. 29A shows tumor volume-time changing curves of LD1-2009-362263 human triple negative breast cancer PDX model treated with antibody-drug conjugate 02-1-LP1. FIG. 29B shows weight-time curves of LD1-2009-362263 human triple negative breast cancer PDX model treated with antibody-drug conjugate 02-1-LP1.

FIG. 30A shows tumor volume-time changing curves of LD1-0060-200770 human cholangiocarcinoma PDX model treated with antibody-drug conjugate 02-1-LP1. FIG. 30B shows weight-time curves of LD1-0060-200770 human cholangiocarcinoma PDX model treated with antibody-drug conjugate 02-1-LP1.

FIG. 31A shows tumor volume-time changing curves of OV-10-0073 human ovarian cancer PDX model treated with antibody-drug conjugate Human IgG-LP1 and 02-1-LP1. FIG. 31B shows weight-time curves of OV-10-0073 human ovarian cancer PDX model treated with antibody-drug conjugate Human IgG-LP1 and 02-1-LP1.

### DETAILED DESCRIPTION

Unless defined otherwise, all numbers used in this specification and claim to indicate content, concentration, ratio, mass, volume, time, temperature, thickness, technical effect, etc., should in any case be understood to be modified by terms “about” or “approximately”. Accordingly, unless indicated to the contrary, the numerical parameters set forth in the following specification and appended claim are approximations. To those skilled in the art, the numerical parameters may vary depending upon the desired properties and effects sought to be obtained by the present disclosure, and each numerical parameter should be interpreted in accordance with the number of significant digits and conventional rounding methods, or as understood by those skilled in the art.

Although the numerical ranges and parameters setting forth the broad scope of the disclosure are approximations, the numerical values set forth in the specific examples are provided as precisely as possible. Any numerical value, however, inherently include certain errors necessarily resulting from the standard deviation found in the respective testing measurements. Every numerical range given throughout this specification will include every narrower numerical range that falls within the broader numerical range, as if the narrower numerical ranges were all expressly written herein.

In ADCs, a linker, as a connecting structure connecting an antibody and a payload, is the key factor for the successful construction of ADCs. Its molecular design and properties are critical determinant factors for ADC efficacy in terms of pharmacokinetics (PK)/pharmaco-dynamics (PD) and therapeutic window. For optimal efficacy, an ideal linker should have the following properties: (1) The linker needs to possess sufficient stability in plasma so that ADCs can circulate in the bloodstream and localize to the tumor site without premature cleavage. Instability of the linker causes premature liberation of the toxic payload and undesired damage to non-target healthy cells, leading to systemic toxicity and adverse effects. (2) The linker needs to possess the ability to be rapidly cleaved and to release free and toxic payload once the ADC is internalized into the target tumor cell. (3) Another property to be considered in the linker design is hydrophobicity. Hydrophobic linkers coupled with hydrophobic payloads often promote aggregation of ADCs. Such molecules are unfavorable in the pursuit of therapeutically useful ADCs and may cause hepatotoxicity or provoke undesired immune response (Kyoji Tsuchikama et al., Antibody-drug conjugates: recent advances in conjugation and linker chemistrie, Protein Cell. 2018 January; 9 (1): 33-46).

### Antibody-Drug Conjugate

The present disclosure provides an antibody-drug conjugate (ADC), or an isomer, an isotopic variant, a pharmaceutically acceptable salt, prodrug, solvate thereof, or combinations thereof, which includes an antibody or antigen-binding fragment thereof, a payload, and a linker of formula I,

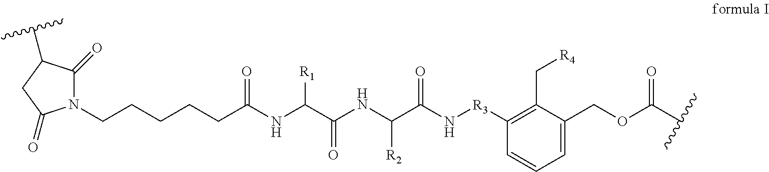

a succinimidyl group of the linker of formula I forms a thioether bond with a thiol group obtained by a reduction of an interchain disulfide chain of the antibody or antigen-binding fragment thereof;
a carbonyl in an ester group of the linker of formula I is connected to an amino group of the payload;
R₁ and R₂ independently are hydrogen, methyl, or isopropyl group;
R₃ represents —(CR₅HCONH)n¹-(CH₂CONH)n²- or a single bond, R₅ is hydrogen or benzyl, n¹ represents an integer of 0 to 2, and n² represents an integer of 0 to 2;
R₄ represents a methylamino group or —(NCH₃COCH₂)n³-NCH₃COCH₃, and n³ represents an integer of 1 to 20.

The term “antibody-drug conjugate” or “ADC” refers to a conjugate of an anti-MUC18/CD44v7/8 antibody or antigen-binding fragment thereof described herein covalently linked to a payload. Generally, the antibody-drug conjugate may include an antibody or antigen-binding fragment thereof, a payload, and optionally a linker between the antibody or antigen-binding fragment thereof and the payload. ADCs may provide therapeutic effects by delivering payloads to MUC18 and/or CD44v7/8 cells targeted by the antibody or antigen-binding fragment thereof, especially MUC18 and/or CD44v7/8 tumor cells. Antibody-drug conjugates can be prepared by various methods known in the art for preparing antibody-drug conjugates.

As used herein, “antibody” refers to a polypeptide of the immunoglobulin (Ig) family that binds with an antigen. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region (abbreviated herein as CL). The light chain constant region is comprised of one domain. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

As used herein, the term “antigen-binding fragment” refers to an antibody fragment including a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multi-specific antibody formed from a portion of an antibody including one or more CDRs, a camelized single domain antibody, a nanobody, a domain antibody, a bivalent domain antibody, or any other antibody fragment that binds to an antigen but does not include a complete antibody structure. An antigen-binding fragment is capable of binding to the same antigen to which the parent antibody or a parent antibody fragment (e.g., a parent scFv) binds.

The term “isomer” refers to compounds that have the same molecular formula but differ in structure, which is also called structural isomer, usually including structural isomers and stereoisomers. Structural isomers refer to isomers caused by differences in the connecting order of atoms in the molecule or different bonding properties, preferably including tautomer. Tautomer refers to functional group isomer resulting from the rapid movement of an atom in two positions in a molecule. Stereoisomers refer to isomers caused by atoms or atomic groups in a molecule that are connected to each other in the same order and bond, but differ in spatial arrangements, preferably including optical isomers. Optical isomers refer to stereoisomers with different optical properties due to the absence of anti-axial symmetry in the molecule, such as enantiomers, diastereomers, racemates and mesomers.

The term “prodrug” refers to a compound obtained by modifying the chemical structure of a drug, which is inactive or less active in vitro, and releases the active drug through enzymatic or non-enzymatic transformation in vivo to exert pharmacological effects. In the present disclosure, a prodrug can be ADC molecule or payload.

The term “payload” includes compounds that are cytotoxic or capable of killing cells upon release from the antibody-drug conjugate, compounds, radionuclides or polypeptides with radiolabels, fluorophores, chromophores, imaging agents and/or metal ions as detection labels or having cell killing effects, compounds, nucleic acids, polypeptides or proteins, enzymes, hormones or nucleic acids that can modulate immune activity in the body (including effects of activation or inhibition).

Under some ideal conditions, in the antibody-drug conjugate, the conjugated payload has little cytotoxicity, or the cytotoxicity thereof is so low that administration of a therapeutically effective dose of the ADC will not cause systemic toxicity in the subject due to the conjugated payload. The payload can be a clinically validated drug for the treatment of a specific disease, or compound, radionuclide, nucleic acid, protein, or polypeptide with acceptable pharmacological activity under conditions of clinical use.

In the present disclosure, a terminal succinimidyl group of formula I and a thiol group obtained by a reduction of an interchain disulfide chain of the antibody or antigen-binding fragment thereof are linked together to form a thioether bond. The succinimidyl group is

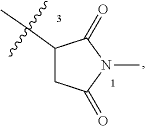 [SMILES: *C1CC(=O)N(C)C1=O]

which forms a thioether bond at position 3 with a thiol moiety after a reduction of an interchain disulfide chain of the antibody or antigen-binding fragment thereof. In the present disclosure, a bond with [symbol] represents a chemical bond connected to other groups.

In the present disclosure, the disulfide bond of the antibody or antigen-binding fragment thereof including interchain disulfide bond and intrachain disulfide bond, preferably, an interchain disulfide chain that is processed, for example, activated to thiol and then bonded to linkers. The amino acid in the antibody or antigen-binding fragment thereof that are chemically bonded to the succinimidyl group in the linker includes one of lysine, histidine, tyrosine and cysteine, or combinations thereof. Optionally, the chemically bonded amino acid in the antibody or antigen-binding fragment thereof is cysteine. In some embodiments, the linker of formula I may be linked to the hinge, variable and/or constant regions of the antibody.

In some embodiments, in the linker of formula I, R₄ represents —(NCH₃COCH₂)n³-NCH₃COCH₃, and n³ represents an integer of 1 to 20. n³ may be, for example, any integer of 1, 2, 3, 4, 5, 6, 7, 8, 9, 10, 11, 12, 13, 14, 15, 16, 17, 18, 19, and 20.

In some embodiments, R₄ represents —(NCH₃COCH₂)n³-NCH₃COCH₃, n³ represents an integer of 8 to 15. In some embodiments, R₄ represents —(NCH₃COCH₂)n³-NCH₃COCH₃, n³ represents an integer of 10 to 12. In some embodiments, R₄ represents a methylamino group.

In the present disclosure, the linker of formula I contains a hydrophilic amino group R₄, which contains a polysarcosine group or a methylamino group, increasing the hydrophilicity of the antibody-drug conjugate. The introduction of hydrophilic amino group is benefit for improvement on hydrophilicity of the ADC conjugated with a hydrophobic payload. The increased hydrophilicity of the ADC molecules is helpful to reduce aggregation of ADC molecules in a preparation process, thereby improving the stability, the uniformity, and the purity of antibody-drug conjugate.

In some embodiments, in the linker of formula I, R₃ represents a single bond.

In some embodiments, R₃ represents —(CR₅HCONH)n¹-(CH₂CONH)n²-, R₅ is benzyl, n¹ represents 1 or 2, and n² represents 1 or 2.

In some embodiments, R₃ represents —CR₅HCONH—, —CH₂CONH—, —CR₅HCONH—CH₂CONH—, —(CR₅HCONH)₂—CH₂CONH—, —CR₅HCONH—(CH₂CONH)₂—, or —(CR₅HCONH)₂—(CH₂CONH)₂—, R₅ is benzyl.

In some embodiments, R₁ is hydrogen. In some embodiments, R₁ is isopropyl.

In some embodiments, R₂ is hydrogen. In some embodiments, R₂ is methyl.

In some embodiments, the linker in the antibody-drug conjugate is selected from the group consisting of

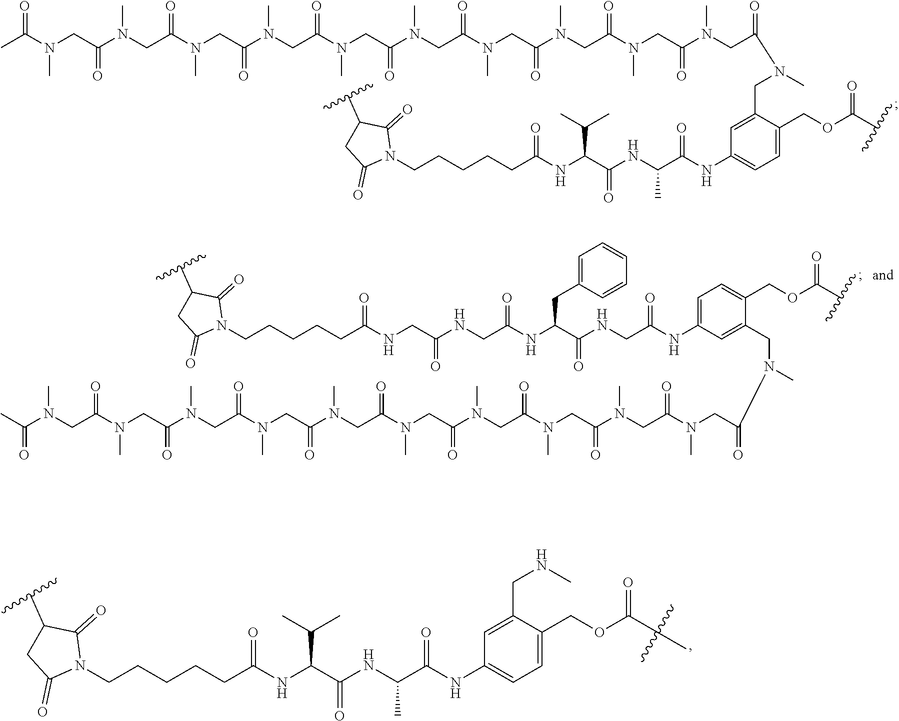 [SMILES: *C(=O)OCc1ccc(NC(=O)[C@H](C)NC(=O)[C@@H](NC(=O)CCCCCN2C(=O)CC(*)C2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *C(=O)OCc1ccc(NC(=O)CNC(=O)[C@H](Cc2ccccc2)NC(=O)CNC(=O)CNC(=O)CCCCCN2C(=O)CC(*)C2=O)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *C(=O)OCc1ccc(NC(=O)[C@H](C)NC(=O)[C@@H](NC(=O)CCCCCN2C(=O)CC(*)C2=O)C(C)C)cc1CNC]

In some embodiments, the payload in the antibody-drug conjugate is a label containing radiolabels, fluorophores, chromophores, imaging agents and/or metal ions as detection labels. The label includes, but is not limited to, chemically synthesized organic compounds, radionuclides, metal complexes or polypeptides. Wherein the radiolabel refers to a labeled compound in which one or several kinds of atoms of the compound molecule are replaced with a radionuclide so that the compound can be identified and used as a tracer, and the radiolabel includes amino acids, polypeptides, proteins, carbohydrates, nucleotides, nucleosides, purines, pyrimidines, steroids, lipid compounds, as well as tumor antigens, hormones, receptors, vitamins and drugs used in medical research. The radionuclide is usually nuclide capable of spontaneously emitting radiation, including but not limited to tritium, iodine 125, iodine 131, sulfur 35, phosphorus 32 and carbon 14. The fluorophore is usually a group including a conjugated double bond, and the fluorophore emits fluorescence when a molecule falls back to a ground state from an excited state. The chromophore refers to an unsaturated group and associated chemical bonds thereof which are contained in a molecule, capable of absorbing light radiation and have transitions. The imaging agent usually refer to radiopharmaceuticals capable of imaging organs, tissues or molecules when introduced into the body in nuclear medicine.

In some embodiments, the payload in the antibody-drug conjugate is nucleic acid which can be ribonucleic acid and/or deoxyribonucleic acid.

In some embodiments, the payload in the antibody-drug conjugate is hormone, growth factor, coagulation factor, and plasminase (e.g., prodrug converting enzymes capable of converting prodrugs to active drugs, ribonucleases).

In some embodiments, the payload in the antibody-drug conjugate is immunomodulator (including cytokines and chemokines that can affect immunity), or agonistic or antagonistic antibodies with biological activity.

In some embodiments, the payload in the antibody-drug conjugate is cytotoxic compound. In some embodiments, the payload in the antibody-drug conjugate has an anti-tumor activity or is an anti-tumor drug. The payload is selected from DNA topoisomerase inhibitor or tubulin inhibitor. The DNA topoisomerase inhibitor can be a topoisomerase I inhibitor or a topoisomerase II inhibitor.

In the present application, the term “topoisomerase inhibitor” usually refers to a compound that inhibits topoiosmerase activity. Compounds known as topoisomerase I inhibitors have activity against topoisomerase I, and topoiosmerase II inhibitors have activity against topoisomerase II. Some compounds have activity against both topoisomerase I and topoisomerase II and are known as topoisomerase I/II inhibitors.

The term “tubulin inhibitor” usually refers to compounds that inhibit the microtubule system of eukaryotic cells, interfere with cell division, and inhibit cell proliferation.

In some embodiments, the payload is camptothecin or derivatives thereof having topoisomerase inhibitory effect. The term “derivative” refers to a compound formed by replacing atoms or atomic groups in the molecule of the parent compound with other atoms or atomic groups and is called a derivative of the parent compound. The term “camptothecin and derivatives thereof” generally includes camptothecin and camptothecin derivatives. Camptothecin exerts its pharmacological effects by irreversibly inhibiting topoisomerase I. The camptothecin derivatives include exatecan, irinotecan, topotecan, lurtotecan, silatecan, etirinotecan pegol, TAS 103, 9-aminocamptothecin, 7-ethylcamptothecin, 10-hydroxycamptothecin, 9-nitrocamptothecin, 10,11-methylenedioxycamptothecin, 9-amino-10,11-methylenedioxycamptothecin, 9-chloro-10,11-methylenedioxycamptothecin, (7-(4-methylpiperazinomethylene)-10,11-ethylenedioxy-20(S)-camptothecin, 7-(4-methylpiperazinomethylene)-10,11-methylenedioxy-20(S)-camptothecin, and 7-(2-N-isopropylamino)ethyl)-(20S)-camptothecin, and stereoisomers, salts and esters thereof. Synthetic methods for camptothecin and analogs or derivatives thereof are known and are summarized and set forth in U.S. Pat. No. 5,244,903, which is herein incorporated by reference in its entirety.

In some embodiments, the payload is auristatin or derivatives thereof, maytansine or derivatives thereof, which have a tubulin inhibitory effect. The term “auristatin or derivatives thereof” usually includes auristatin F and auristatin F derivatives. The auristatin F derivatives include monomethyl auristatin E (MMAE) and monomethyl auristatin F (MMAF). The term “maytansine or derivatives thereof” usually includes maytansine and maytansine derivatives. The maytansine derivatives include maytansine DM1, maytansine DM2, and maytansine DM4.

In some embodiments, the payload is exatecan, a camptothecin derivative, which as a topoisomerase inhibitor, can act throughout the cell cycle and has strong penetration and good therapeutic effect on slow-growing solid tumors. In addition, the number of intracellular targets is much less than that of targets of tubulin inhibitors, thus a better killing effect can be achieved when ADC molecules carry the same amount of payload into cells. Exatecan molecules are not substrates of P-gp, which is beneficial to reduce or alleviate the problem of drug resistance.

In some embodiments, the payload is exatecan of formula II, which is connected to the linker by a nitrogen atom of an amino group on a cyclohexane ring thereof,

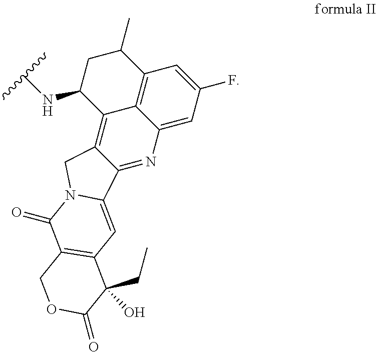 [SMILES: *N[C@H]1CC(C)c2cc(F)cc3nc4c(c1c23)Cn1c-4cc2c(c1=O)COC(=O)[C@]2(O)CC]

The exatecan molecule is rigid in structure and has a poor hydrophilicity, thus it is easy to cause polymerization between ADC molecules when it is connected to GGFG tetrapeptide linker commonly used in the prior art to prepare ADC, which does not meet the development requirements of ADC drugs (Bioorg. Med Chem. Lett. 26 (2016) 1542-1545). Therefore, the selection and matching of the linker and the payload have impacts on the safety and stability of ADC drugs.

Without being bound by any theory, in the antibody-drug conjugates provided by the present disclosure, due to the multiple hydrophilic groups in the linker, the hydrophilicity of the linker-payload structure is improved, and the aggregation and precipitation of ADC molecules caused by the hydrophobic payload can be reduced to a certain extent.

After ADC molecule is endocytosed into a cell, compounds of the payload or the linker (or part of the linker)-payload structure are released depending on whether the linker is degraded or not. In some embodiments, an amino group on a cyclohexane ring of exatecan of formula II is bonded to the carbonyl group in the ester group of the linker of formula I and forms a linker-payload structure including carbamate. Without being bound by any theory, in the linker-payload structure provided by the present disclosure, after the ADC molecule is endocytosed into the cell, the linker is cleaved by cathepsin (for example, Cathepsin B) to form an intermediate or active metabolite of formula IV below,

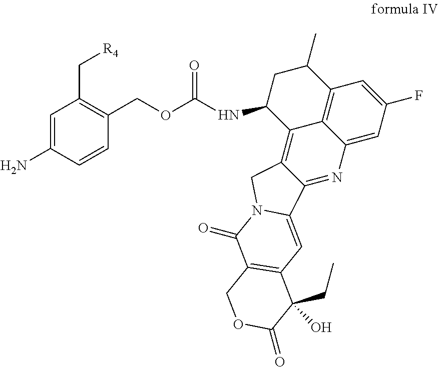

wherein, R₄ represents a methylamino group or —(NCH₃COCH₂)n³-NCH₃COCH₃, and n³ represents an integer of 1 to 20.

The PABC (para-amino benzyloxycarbonyl) group in the intermediate or the active metabolite of formula IV then undergoes 1,6-elimination to release exatecan (see, Angew. Chem. Int. Ed. 2015, 54, 7492-7509). Therefore, the linker-payload structure in the ADC molecule provided by the present disclosure has good in vivo stability and biological activity.

Without being bound by any theory, the cleavage site in the linker-payload structure may be an amide bond in the linker, for example, the amide bond between a carbon atom where the substituent represented by R₂ is located and a group represented by R₃, or an amide bond in the group represented by R₃.

In some embodiments, in the antibody-drug conjugate, a ratio of the molecule number of the conjugated payload to each molecule of antibody or antigen-binding fragment, i.e., drug-to-antibody ratio, or DAR, is 1 to 10. In some embodiments, DAR is 1˜2, 2˜4, 4˜6, 2˜8, 4˜8, 4˜10, 6˜10, 7˜10, or 8˜10, an exemplary value of DAR is 4, 6, 7.8, 9.2, or 9.92.

DAR represents an average value of the number of the conjugated payload or drug molecules per antibody molecule, that is, an average number of conjugated drug molecules. In the antibody-drug conjugate, DAR is a key factor having an influence on the efficacy and safety thereof. The production of the antibody-drug conjugate is carried out by specifying reaction conditions such as the amounts of starting materials and reagents used for reaction, so as to attain a constant number of conjugated payload molecules. A mixture containing different numbers of conjugated payload molecules is usually obtained when the antibody-drug conjugate is prepared. Unless otherwise specified, in the present disclosure, the DAR is defined as an average value, i.e., the average number of conjugated payload or drug molecules.

In some embodiments, the antibody-drug conjugate includes any one of the following structures:

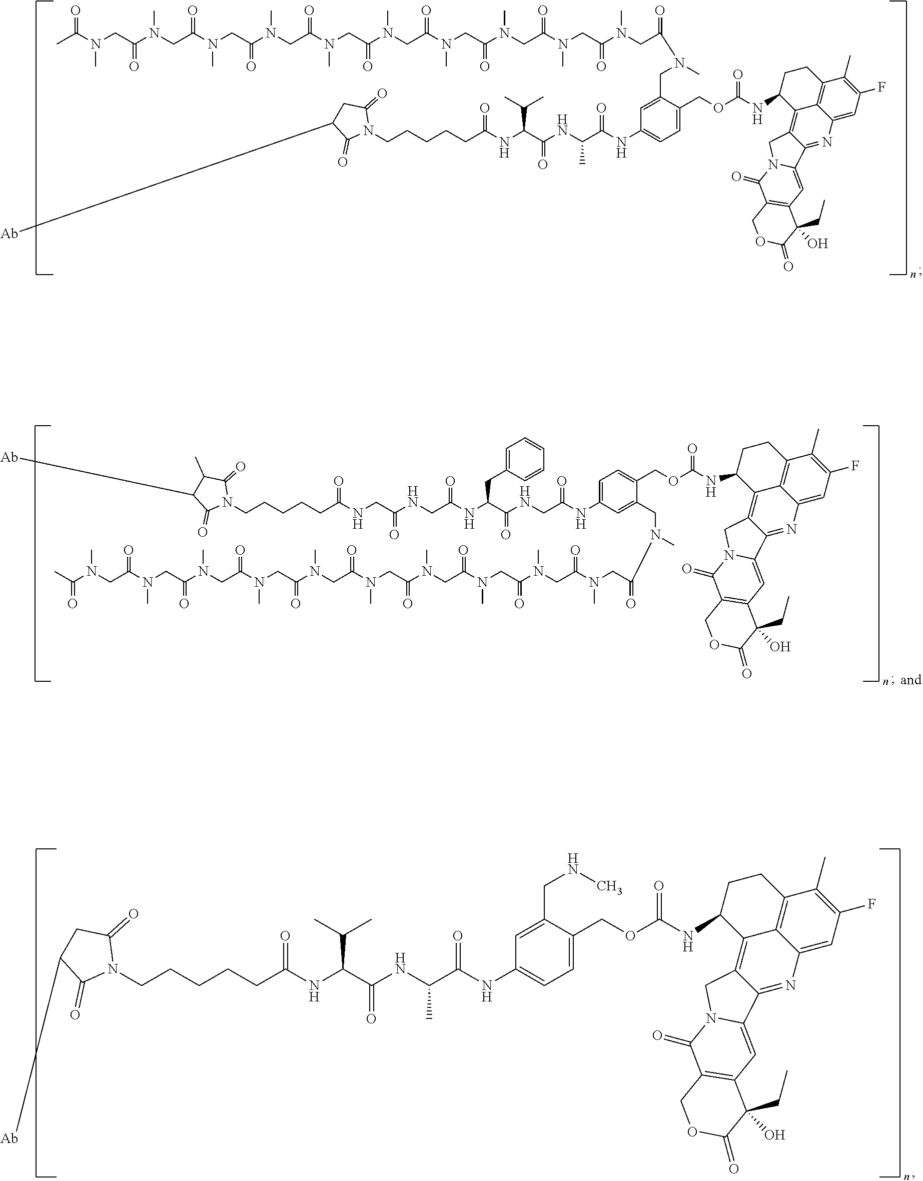

Ab represents an antibody or antigen-binding fragment thereof; n is the same as the DAR.

The antibody or antigen-binding fragment thereof is connected to the linker by a reduced reactive thiol group, optionally, the disulfide bond in a hinge region of the antibody or antigen-binding fragment thereof is reduced to a reactive thiol group and then is connected to a linker.

In some embodiments, the antibody or antigen-binding fragment thereof in the antibody-drug conjugate is targeted to MUC18. MUC18, also known as CD146 or melanoma cell adhesion molecule (MCAM), is a transmembrane glycoprotein that functions primarily in cell adhesion. It is expressed at detectable levels in endothelial cells within vascular tissue, including vascular smooth muscle. Notably, MUC18 is overexpressed in human malignant melanoma, particularly in metastatic lesions and advanced primary tumors.

In the present disclosure, the anti-MUC18 antibody or antigen-binding fragment thereof includes a heavy chain (H) and a light chain (L), the CDRs of the heavy chain and the light chain are shown in Table 1.

TABLE 1
CDR sequence (numbered by IMGT scheme)

| Antibody | | | | |
|---|---|---|---|---|
| Code | H/L | CDR1 | CDR2 | CDR3 |
| CL070336 | H | GYSITSGYS | MHYSATP | SRSLNWDVLDY |
| | | (SEQ ID NO: 1) | (SEQ ID NO: 11) | (SEQ ID NO: 23) |
| | L | KSVRTSGYNY | LAS | QHSREFPFT |
| | | (SEQ ID NO: 34) | | (SEQ ID NO: 46) |
| CL070335 | H | GYSITSGYS | IHYSTTT | SRSLNWDVLDY |
| | | (SEQ ID NO: 1) | (SEQ ID NO: 12) | (SEQ ID NO:23) |
| | L | KSVRTSGYSY | LAS | QHTREFPFT |
| | | (SEQ ID NO: 35) | | (SEQ ID NO: 47) |
| CL070333 | H | GFTFTGYY | IYPGNVNT | ARSGYFASSYEFAY |
| | | (SEQ ID NO: 2) | (SEQ ID NO: 13) | (SEQ ID NO: 24) |
| | L | ENIYSY | NAK | QHHYGTPLT |
| | | (SEQ ID NO: 36) | | (SEQ ID NO: 48) |
| CL070319 | H | GYTFTEYA | ITAYSGDA | ARGLGRPNYGMDY |
| | | (SEQ ID NO: 3) | (SEQ ID NO: 14) | (SEQ ID NO: 25) |
| | L | QSLLNSRYQKNY | FAS | QQHYGTPFT |
| | | (SEQ ID NO: 37) | | (SEQ ID NO: 49) |
| CL070321 | H | GYTFTDYN | INPNNGGT | ARSGYDDYYAFDY |
| | | (SEQ ID NO: 4) | (SEQ ID NO: 15) | (SEQ ID NO: 26) |
| | L | SSVGY | STS | HQWSSYPST |
| | | (SEQ ID NO: 38) | | (SEQ ID NO: 50) |
| CL070320 | H | GYTFTDYN | INPNNGGT | ARSGYDNYYALDY |
| | | (SEQ ID NO: 4) | (SEQ ID NO: 15) | (SEQ ID NO: 27) |
| | L | STVGY | STS | HQWSSYPCT |
| | | (SEQ ID NO: 39) | | (SEQ ID NO: 51) |
| CL070324 | H | GYTFTDYN | INPNNGAT | TRSGYDDYYALDY |
| | | (SEQ ID NO: 4) | (SEQ ID NO: 16) | (SEQ ID NO: 28) |
| | L | SSVGY | STS | HQWSNYPST |
| | | (SEQ ID NO: 38) | | (SEQ ID NO: 52) |
| CL070341 | H | GFTFSTYG | INSRASYT | ARHRRDWDSYYAMDY |
| | | (SEQ ID NO: 5) | (SEQ ID NO: 17) | (SEQ ID NO: 29) |
| | L | QSVLYNSNQKNY | WAS | HQYLSSWT |
| | | (SEQ ID NO: 40) | | (SEQ ID NO: 53) |
| CL070350 | H | GFTFNNYG | INRGGSYT | ARHRRDWDSYYAMDY |
| | | (SEQ ID NO: 6) | (SEQ ID NO: 18) | (SEQ ID NO: 29) |
| | L | RSVLYNSNQKNY | WAS | HQYLSSWT |
| | | (SEQ ID NO: 41) | | (SEQ ID NO: 53) |
| CL070349 | H | GYSITSGFY | INFGGTN | ARGGNDGGFTY |
| | | (SEQ ID NO: 7) | (SEQ ID NO: 19) | (SEQ ID NO: 30) |
| | L | SSVSSTY | RTS | HQWSGYPYT |
| | | (SEQ ID NO: 42) | | (SEQ ID NO: 54) |
| CL070348 | H | GYSITSDYA | ITYSGST | ARRIRRRTGDWYFDV |
| | | (SEQ ID NO: 8) | (SEQ ID NO: 20) | (SEQ ID NO: 31) |
| | L | QNLLNSVNQKNF | WAS | QNDYIYPYT |
| | | (SEQ ID NO: 43) | | (SEQ ID NO: 55) |
| CL070370 | H | GYTFTDYS | INTETGEP | VSYDYDAVSY |
| | | (SEQ ID NO: 9) | (SEQ ID NO: 21) | (SEQ ID NO: 32) |
| | L | QYVGAA | WAS | QQYGNYRT |
| | | (SEQ ID NO: 44) | | (SEQ ID NO: 56) |
| J253 | H | GYTFTSYD | IFPGDGST | ARYHFFDN |
| | | (SEQ ID NO: 10) | (SEQ ID NO: 22) | (SEQ ID NO: 33) |
| | L | KSLLYKDGKTY | LMS | QQLVEYPYT |
| | | (SEQ ID NO: 45) | | (SEQ ID NO: 57) |

As used herein, the term “CDR” or “complementarity determining region” is intended to mean the non-contiguous antigen combining sites within the variable region of a heavy chain and/or a light chain. CDR residue numbering follows the nomenclature of IMGT, see, Lefranc M. P. et al., Dev. Comp. Immunol., 27:55-77 (2003), where the definitions include overlapping or subsets of amino acid residues when compared against each other. Nevertheless, application of any one of the definitions to refer to a CDR of an antibody or antigen-binding fragment thereof or grafted antibodies or variants thereof is intended to be within the scope of the term as defined and used herein.

In some embodiments, the anti-MUC18 antibody or antigen-binding fragment thereof includes heavy chain CDRs and light chain CDRs in antibodies coded CL070336, CL070335, CL070333, CL070319, CL070321, CL070320, CL070324, CL070341, CL070350, CL070349, CL070348, CL070370, or J253.

In some embodiments, the anti-MUC18 antibody or antigen-binding fragment thereof includes HCDR1 (heavy chain CDR1) with amino acid sequence as shown in SEQ ID NO: 4, HCDR2 (heavy chain CDR2) with amino acid sequence as shown in SEQ ID NO: 16, HCDR3 (heavy chain CDR3) with amino acid sequence as shown in SEQ ID NO: 28; and LCDR1 (light chain CDR1) with amino acid sequence as shown in SEQ ID NO: 38, LCDR2 (light chain CDR2) with amino acid sequence STS, LCDR3 (light chain CDR3) with amino acid sequence as shown in SEQ ID NO: 52.

In some embodiments, the anti-MUC18 antibody or antigen-binding fragment thereof includes a heavy chain variable region (VH) that is at least 85% or 90% identical to any of the VHs of reference antibody sequences shown in Table 2, and/or, and a light chain variable region (VL) that is at least 85% or 90% identical to any of the VLs of the reference antibody sequences shown in Table 2.

The identity can be determined using an algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into NBLAST and XBLAST programs (version 2.0) of Altschul, et al., J. Mol. Biol.215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul, et al., Nucleic Acids Res. 25 (17): 3389-3402, 1997. When utilizing BLAST and Gapped BLAST programs, the default parameters of the respective programs (e.g., XBLAST and NBLAST) can be used.

In some embodiments, in framework regions, no mutations exist or mutations that do not affect the binding of the antibody variable region to the antigen exist. The mutations that do not affect the binding of the antibody variable region to the antigen may increase the binding affinity of the antibody to the antigen or keep it approximately unchanged. In some embodiments, anti-MUC18 or CD44v7/8 antibodies or antigen-binding fragments thereof further include conservatively modified variants. The conservatively modified variants include individual substitutions, deletions or additions to the polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

TABLE 2
Reference antibody sequences

| Antibody Code | VH | VL | Antibody Code | VH | VL |
|---|---|---|---|---|---|
| CL070336 | SEQ ID NO: 58 | SEQ ID NO: 59 | CL070341 | SEQ ID NO: 72 | SEQ ID NO: 73 |
| CL070335 | SEQ ID NO: 60 | SEQ ID NO: 61 | CL070350 | SEQ ID NO: 74 | SEQ ID NO: 75 |
| CL070333 | SEQ ID NO: 62 | SEQ ID NO: 63 | CL070349 | SEQ ID NO: 76 | SEQ ID NO: 77 |
| CL070319 | SEQ ID NO: 64 | SEQ ID NO: 65 | CL070348 | SEQ ID NO: 78 | SEQ ID NO: 79 |
| CL070321 | SEQ ID NO: 66 | SEQ ID NO: 67 | CL070370 | SEQ ID NO: 80 | SEQ ID NO: 81 |
| CL070320 | SEQ ID NO: 68 | SEQ ID NO: 69 | J253 | SEQ ID NO: 82 | SEQ ID NO: 83 |
| CL070324 | SEQ ID NO: 70 | SEQ ID NO: 71 | / | / | / |

In some embodiments, the anti-MUC18 antibody or antigen-binding fragment thereof includes the VH that is at least 85%, 90%, 92%, 94%, 95%, 96%, 97%, 98%, 99% identical to the VH shown in Table 3; and the VL that is at least 85%, 90%, 92%, 94%, 95%, 96%, 97%, 98%, 99% identical to the VL shown in Table 3. The antibodies shown in Table 3 are humanized sequences of CL070324 and J253, respectively.

TABLE 3
Humanized antibody sequences

| Antibody Code | VH | VL |
|---|---|---|
| Humanized CL070324 (02-1) | SEQ ID NO: 84 | SEQ ID NO: 85 |
| Humanized hJ253-03-1 | SEQ ID NO: 86 | SEQ ID NO: 87 |
| Humanized hJ253-03-7 | SEQ ID NO: 88 | SEQ ID NO: 89 |

In some embodiments, the anti-MUC18 antibody or antigen-binding fragment thereof includes heavy chain variable region with amino acid sequence as shown in SEQ ID NO: 84, and light chain variable region with amino acid sequence as shown in SEQ ID NO: 85.

In some embodiments, the anti-MUC18 antibody or antigen-binding fragment thereof is Humanized CL070324, Humanized hJ253-03-1, or Humanized hJ253-03-7, or conservative variants thereof.

Wherein the CDR sequences and antibody variable region sequences of the anti-MUC18 antibodies shown in SEQ ID NO: 1˜89 have been recorded in U.S. Patent US2022041749 (SEQ ID NOs: 8˜90, SEQ ID NOs: 1˜6), which is incorporated herein by reference in its entirety.

In some embodiments, the antibody or antigen-binding fragment thereof is targeted to CD44v7/8. CD44, also known as HCAM (homing cell adhesion molecule), Pgp-1 (phagocytic glycoprotein-1), Hermes antigen, lymphocyte homing receptor, ECM-III, or HUTCH-1, is a cell-surface glycoprotein that functions primarily in cell adhesion and cell-cell interactions. Several splice variants of CD44 are known, including CD44 v7/8, which is a variant comprising exon 7 and exon 8. CD44 is a protein receptor for hyaluronic acid and has further been shown to bind or otherwise interact with osteopontin, collagens, matrix metalloproteinases, and other similar ligands.

In some embodiments, the anti-CD44 v7/8 antibody or antigen-binding fragment thereof specifically binds to a binding peptide in human CD44 v7/8, wherein the binding peptide includes an amino acid sequence shown as QAGRRMDMDSSHSIT (SEQ ID NO: 90).

In some embodiments, the anti-CD44 v7/8 antibody or antigen-binding fragment thereof does not bind to a binding peptide with an amino acid sequence of PISHPMGRGHQAGRR (SEQ ID NO: 91) and/or SHSITLQPTANPNTG (SEQ ID NO: 92) in human CD44 v7/8.

In some embodiments, the anti-CD44 v7/8 antibody or antigen-binding fragment thereof includes a heavy chain containing HCDR1 with amino acid sequence as shown in SEQ ID NO: 93, HCDR2 with amino acid sequence as shown in SEQ ID NO: 94, HCDR3 with amino acid sequence as shown in SEQ ID NO: 95; and a light chain containing LCDR1 with amino acid sequence as shown in SEQ ID NO: 96, LCDR2 with amino acid sequence RAN, LCDR3 with amino acid sequence as shown in SEQ ID NO: 97. The CDR sequences of anti-CD44 v7/8 antibody or antigen-binding fragment thereof are shown in Table 4:

TABLE 4
CDR sequences (numbered by IMGT scheme)

| Antibody | | CDR1 | CDR2 | CDR3 |
|---|---|---|---|---|
| HIS1H2-2 | Heavy | GYTFTNYW | IYPSDSYT | TKWGDSDY |
| | chain | (SEQ ID | (SEQ ID | (SEQ ID |
| | | NO: 93) | NO: 94) | NO: 95) |
| | Light | QDINRY | RAN | LQYDEFPFT |
| | chain | (SEQ ID | | (SEQ ID |
| | | NO: 96) | | NO: 97) |

In some embodiments, the anti-CD44 v7/8 antibody or antigen-binding fragment thereof includes the VH that is at least 85% or 90% identical to the VH of the reference antibody sequences shown in Table 5, and/or, the VL that is at least 85% or 90% identical to the VL of the reference antibody shown in Table 5.

In some embodiments, the anti-CD44 v7/8 antibody or antigen-binding fragment thereof includes the VH that is at least 85%, 90%, 92%, 94%, 95%, 96%, 97%, 98%, 99% identical to the VH of HIS1H2-2a or HIS1H2-2; and the VL that is at least 85%, 90%, 92%, 94%, 95%, 96%, 97%, 98%, 99% identical to the VL of HIS1H2-2a or HIS1H2-2, which are humanized sequence of the reference antibody shown in Table 5.

TABLE 5
Heavy/light chain variable region sequences
of anti-CD44 v7/8 antibody

| Antibody Code | VH | VL |
|---|---|---|
| Reference antibody HIS1H2-2 | SEQ ID NO: 98 | SEQ ID NO: 99 |
| Humanized antibody HIS1H2-2a | SEQ ID NO: 100 | SEQ ID NO: 101 |
| Humanized antibody HIS1H2-2 | SEQ ID NO: 100 | SEQ ID NO: 104 |

In some embodiments, the anti-CD44 v7/8 antibody or antigen-binding fragment thereof includes heavy chain variable region with amino acid sequence as shown in SEQ ID NO: 100, and light chain variable region with amino acid sequence as shown in SEQ ID NO: 101 or SEQ ID NO: 104, or conservative variants thereof.

The binding peptide sequences, the unbound peptide sequences, the CDR sequences and the variable region sequences of the anti-CD44 v7/8 antibodies or antigen-binding fragment thereof shown in SEQ ID NO: 90˜101 have been recorded in PCT Patent Application WO2020159754 (SEQ ID Nos: 2˜14), which is incorporated by reference in its entirety.

The antibody-drug conjugates provided herein show excellent killing activity on tumor cells, and extremely high killing activity on tumor cells with high expression of MUC18 or CD44v7/8.

The antibody-drug conjugates provided by the present disclosure show excellent anti-tumor activity. In some embodiments, the antibody-drug conjugates show excellent anti-tumor activity on subjects suffering neoplastic diseases with different expression levels (low, moderate and high) of MUC18. The expression level of MUC18 may be measured by H-Score approach known to those skilled in the art. The score ranges from 0 to 300, and the higher the score, the higher the expression. For example, low (H-Score=10-99), moderate (H-Score=100-199), high (H-Score=200-300). In addition, the antibody-drug conjugates show no obvious adverse effects in vivo.

It should be noted that the antibody-drug conjugate of the present disclosure may absorb water, retain the adsorbed water, or become a hydrate due to being left in the atmosphere or recrystallized, and such a water-containing compound and salts thereof are also included in the present disclosure. In addition, compounds of isotope variants labeled with various radioactive or non-radioactive isotopes are also included in the present disclosure. More than one of the atoms constituting the antibody-drug conjugate of the present disclosure may also contain atomic isotopes in unnatural proportions. As an atomic isotope, for example, deuterium (2H), tritium (3H), iodine-125 (125I), and carbon-14 (14C), etc., may be exemplified. In addition, the compounds of the present disclosure may be radiolabeled with radioisotope such as tritium (3H), iodine-125 (125I), or carbon-14 (14C). Radiolabeled compounds can be used as therapeutic or prophylactic agents, research reagents such as test reagents, and diagnostic agents, e.g., in vivo imaging diagnostics. All isotopic variants of the antibody-drug conjugate of the present disclosure, whether radioactive or not, are included within the scope of the present disclosure.

### Preparation Method

The present disclosure provides a preparation method of antibody-drug conjugate, or an isomer, an isotopic variant, a pharmaceutically acceptable salt, prodrug, solvate thereof, or combinations thereof, and a pharmaceutically acceptable excipient, which includes the following steps: reducing the antibody or antigen-binding fragment thereof such that interchain disulfide bonds thereof are at least partially reduced, and reacting with a carbon atom at position 3 of maleimide-N-yl of the linker of formula III in linker-payload,

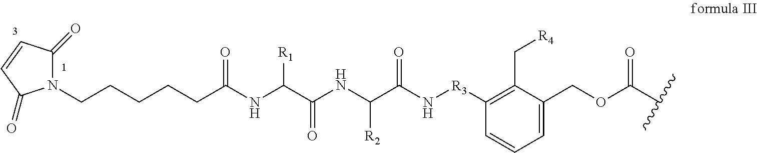

A carbonyl group in an ester group of the linker of formula III is connected to an amino group of the payload in the antibody-drug conjugate;
R₁ and R₂ are independently selected from hydrogen, methyl and isopropyl group;
R₃ represents —(CR₅HCONH)n¹-(CH₂CONH)n²- or a single bond, R₅ is hydrogen or benzyl, n¹ represents an integer of 0 to 2, and n² represents an integer of 0 to 2;
R₄ represents a methylamino group or —(NCH₃COCH₂)n³-NCH₃COCH₃, and n³ represents an integer of 1 to 20.

In antibody-drug conjugate, the interchain disulfide bond is reduced to a thiol group and then reacts with the reactive group of the linker represented by formula III in the linker-payload. In many practical cases, linkers of formula III with a payload are connected to the same antibody or antigen-binding fragment thereof having a reactive thiol group. In some embodiments, the antibody or antigen-binding fragment thereof reacts with a reducing agent such as dithiothreitol (DTT), 2-mercaptoethanol, or tris(2-carboxyethyl) phosphine hydrochloride (TCEP), causing disulfide bond of the antibody or antigen-binding fragment thereof to form a reactive thiol group. The amount of the reducing agent can be 0.3-10 times the molar equivalent of the antibody or antigen-binding fragment thereof, for example, 1-10, 3-10, 5-10, 7-10 times the molar equivalent of the antibody or antigen-binding fragment thereof.

In some embodiments, the method further includes: reacting the antibody or antigen-binding fragment thereof with a reducing agent in a buffer solution containing a chelating agent, followed by an addition of a linker-payload solution to carry out the reaction. The linker-payload is particularly a compound formed by the bonding of the linker of formula III and the payload, wherein the amino group (primary amino group) in the payload is connected to the carbonyl group in the ester group of the linker of formula III. In the linker-payload, the payload is selected from the payloads described in the [Antibody-Drug Conjugates] section. The term “chelating agent” refers to a complex capable of forming a complex with a cyclic structure by coordinative bonding with a metal atom or ion.

In some embodiments, the reducing agent reacts with the antibody or antigen-binding fragment thereof in a buffered solution containing a chelating agent, resulting in an antibody or antigen-binding fragment thereof with partially or fully reduced interchain disulfide bonds. The chelating agent includes, but is not limited to, ethylenediaminetetraacetic acid (EDTA) and diethylenetriaminepentaacetic acid (DTPA). The chelating agent is used at a concentration of 1 mM˜20 mM, for example, 2 mM˜20 mM, 5 mM˜20 mM, 8 mM˜20 mM, 1 mM˜15 mM, or 1 mM˜10 mM. The components of the buffer solution may be buffer salts commonly used in the art, such as sodium phosphate, sodium borate, sodium acetate, or similar buffer salts.

The reaction of the antibody or antigen-binding fragment thereof with the reducing agent is carried out under adjusted pH value. In some embodiments, the antibody or antigen-binding fragment thereof reacts with the reducing agent under pH 5˜9, optionally, 6˜8, 6˜7, 6.5˜7.5, or 7˜8. For example, the reaction is carried out under pH about 7. The pH of the reaction solution can be adjusted using either acidic or basic chemicals, and exemplary acidic or basic chemicals include acetic acid, hydrochloric acid, phosphoric acid, sulfuric acid, sodium bicarbonate, sodium carbonate, sodium hydroxide, and triethylamine.

The reaction of antibody or antigen-binding fragment thereof and the reducing agent is carried out under regulated temperature, and an exemplary reaction temperature is −10˜40° C., −10˜10° C., 5˜40° C., 10˜40° C., 25˜40° C., 30˜40° C., or 35˜38° C., for example, about 37° C.

The linker-payload may be dissolved in an organic solvent selected from any one of dimethyl sulfoxide (DMSO), dimethylformamide (DMF), dimethylacetamide (DMA), and N-methyl-2-pyrrolidone (NMP), or combinations thereof.

In some embodiments, a solution of the linker-payload is added to a buffer solution of the antibody or antigen-binding fragment thereof that has been reduced or has reactive thiol groups in an amount of a volume ratio of 1% to 20%, based on the volume of the buffer solution of the antibody or antigen-binding fragment thereof. In some embodiments, the volume ratio of the added solution of the linker-payload is 1˜20%, 2˜20%, 5˜20%, 10˜20%, 15˜20%, 1˜18%, 1˜15%, 1˜13%, 1˜10%, or 5˜15%, based on the volume of buffer solution of the antibody or antigen-binding fragment thereof.

In some embodiments, a molar ratio of the linker-payload to the antibody or antigen-binding fragment thereof is 4˜20, optionally, 8˜20. In some embodiments, the molar ratio of the linker-payload to the antibody or antigen-binding fragment thereof is 10˜20, 14˜20, 16˜20, or 18˜20.

In some embodiments, a temperature at which the antibody or antigen-binding fragment thereof reacts with the linker-payload is −10˜40° C. or 0˜37° C. In some embodiments, a reaction temperature is −10˜10° C., 5˜40° C., 5˜37° C., 10˜37° C., 10˜25° C., or 15˜30° C.

In some embodiments, a time for the antibody or antigen-binding fragment thereof to react with linker-payload is 0.5˜2 hours. In some embodiments, the time for the antibody or antigen-binding fragment thereof to react with linker-payload is 0.5˜1.75 hours, 0.5˜1.5 hours, 0.5˜1.25 hours, 0.75˜2 hours, or 1˜2 hours.

The reaction can be terminated by inactivating the unreacted linker-payload using a thiol-containing reagent. The thiol-containing reagent includes, but is not limited to, cysteine or N-acetyl-(L)-cysteine (NAC). More specifically, a thiol-containing reagent with a molar equivalent of 1-2 times that of the linker-payload is added to the reaction solution and incubated at room temperature (10-30° C.) for 10-30 minutes, as such, the reaction can be terminated.

In the case where the antibody or antigen-binding fragment thereof has a thiol group, the antibody-drug conjugate may also be obtained by reaction of compounds using known methods (e.g., available by the method described in patent publication US2016/297890 (e.g., available by the methods described in paragraphs [0336] to [0374]). The antibody or antigen-binding fragment thereof having a thiol group can be obtained by methods well known to those skilled in the art (Hermanson, G. T, Bioconjugate Techniques, pp. 56-136, pp. 456-493, Academic Press (1996)).

The antibody-drug conjugate provided by the present disclosure can be obtained by the above preparation method. In some embodiments, the prepared antibody-drug conjugate is subjected to a purification process including, but not limited to, gel filtration, e.g., a purification using a gel column.

### Kit

The present disclosure provides a kit including the antibody-drug conjugates described above, or an isomer, an isotopic variant, a pharmaceutically acceptable salt, prodrug, solvate thereof, or combinations thereof. The kit may further include instructions for use of the antibody-drug conjugates described herein in the methods of the disclosure, such as, in the methods for treating neoplastic diseases.

The kit may further include a container. Suitable containers include, for example, bottles, vials (e.g., dual chamber vials), syringes (such as single or dual chamber syringes) and test tubes. The container may be formed from a variety of materials such as glass or plastic. The container holds the formulation. The container holding the formulation may be a single-use vial or a multi-use vial, which allows for repeat administrations of the reconstituted formulation.

The kit may further include a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating neoplastic diseases (e.g., cancer) in a subject. The kit may further include other materials desirable from a commercial, therapeutic, and user standpoint, including other buffers, diluents, filters, needles, syringes, and package inserts with instructions for use.

### Pharmaceutical Composition

The present disclosure provides a pharmaceutical composition which includes the antibody-drug conjugate described above, or an isomer, an isotopic variant, a pharmaceutically acceptable salt, prodrug, solvate thereof, or combinations thereof, and a pharmaceutically acceptable excipient.

The antibody-drug conjugate or the pharmaceutical composition of the present disclosure can be administered in a suitable manner according to the specific applicable form, physicochemical characteristics, etc. of the pharmaceutically acceptable excipients. In some embodiments, the pharmaceutical composition may be formulated in a form of a freeze-dried formulation or a liquid formulation, which may contain appropriate formulation additives in the art. For example, the above-mentioned pharmaceutical composition typically contains more than one pharmaceutical carrier, for example, sterile liquid such as water and oil (including petroleum, oils of animal, vegetable, or synthetic origin (e.g., peanut oil, soybean oil, mineral oil, and sesame oil, etc.)). In the case of intravenous administration of the above pharmaceutical composition, water is a more representative carrier. In addition, saline solution, aqueous glucose, and glycerol solution may also be used as a liquid carrier, especially for an injectable solution. Suitable pharmaceutical excipients are known in the art. The above pharmaceutical composition may also contain a trace amount of wetting agent, emulsifier, or pH buffering agent as required. The administration mode of the pharmaceutical composition is usually parenteral administration, which can be intradermal, intramuscular, intraperitoneal, intravenous or subcutaneous injection, but is not limited thereto, for example, the pharmaceutical composition may be administered by infusion or bolus injection. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The pharmaceutical composition of the present disclosure may be a pharmaceutical composition containing only the antibody-drug conjugate of the present application, or a pharmaceutical composition containing the antibody-drug conjugate and at least one other therapeutic agent (e.g., cancer therapeutic agent). In some embodiments, the antibody-drug conjugate of the present disclosure may also be administered together with other cancer therapeutic agents to enhance the anticancer effect. Other anticancer agents used for this purpose may be administered to the individual concurrently, separately or sequentially with the antibody-drug conjugate, or may be administered at varying intervals. Exemplary other cancer therapeutic agents may be, for example, paclitaxel, cisplatin, vinblastine, etc., but not limited thereto, as long as they have antitumor activity.

With regard to the present disclosure, the active agent, or pharmaceutical composition comprising the same, can be administered to the subject via any suitable route of administration. For example, the active agent can be administered to a subject via parenteral, nasal, oral, pulmonary, topical, vaginal, or rectal administration. The following discussion on routes of administration is merely provided to illustrate various embodiments and should not be construed as limiting the scope in any way.

In some embodiments, the antibody-drug conjugate of the present disclosure is administered to the subjects. As used herein, the terms “subject” refers to human and non-human animals. Non-human animals include all vertebrates, such as mice, rats, rabbits, cats, dogs, pig, monkey, chimpanzee, gorilla, and the like. In some embodiments, the subject is human.

### Methods of Use

The present disclosure provides use of the antibody-drug conjugate, the antibody-drug conjugate prepared by the above method, pharmaceutical composition including the antibody-drug conjugate, or the kit provided herein in the manufacture of a therapeutic agent for diagnosis, prevention, and treatment of neoplastic disease.

The neoplastic disease includes benign tumors and malignant tumors (e.g., cancer). In some embodiments, the benign tumors and malignant tumors express MUC18 and/or CD44v7/8.

The type of neoplastic disease to which the antibody-drug conjugate is applied is not limited to the above-mentioned cancer cells, as long as it is a cancer cell that expresses a protein recognizable by the antibody or antigen-binding fragment thereof in the antibody-drug conjugate. In some embodiments, the neoplastic disease is a solid tumor expressing MUC18 and/or CD44v7/8.

In some embodiments, the neoplastic disease includes melanoma, pharyngeal cancer, triple-negative breast cancer, esophageal adenocarcinoma, esophagus squamous cell carcinoma, liver cancer, ovarian cancer, cervical cancer, urothelial cancer, bladder neuroendocrine tumor, small cell lung cancer, non-small cell lung cancer, cutaneous squamous cell carcinoma, cholangiocarcinoma, metastatic pancreatic carcinoma, lung squamous cell carcinoma, head and neck squamous cell carcinoma and/or esophageal squamous cell carcinoma.

The present disclosure provides use of the antibody-drug conjugate, the antibody-drug conjugate prepared by the above method, pharmaceutical composition including the antibody-drug conjugate, or the kit in the manufacture of a therapeutic agent targeting MUC18 and/or CD44v7/8.

The present disclosure provides a method of diagnosing, preventing, and treating neoplastic diseases, comprising administering to a subject a therapeutically effective amount of a therapeutic agent described above.

As used herein, the term “therapeutically effective amount” refers to that amount of active ingredient, i.e., ADCs described herein that is sufficient to induce the intended effect, including but not limited to disease treatment, as defined herein. Therapeutic doses of antibody-drug conjugate vary depending on the particular condition being treated, severity of the condition, parameters of individual patient (including age, physical condition, physique, sex, and weight), duration of treatment, nature of the concomitant therapy (if any), the particular route of administration, and the knowledge of the health practitioner. In some embodiments, the dosage of the above-mentioned antibody-drug conjugate can be determined empirically in individuals who have been given one or more administrations of the antibody or antigen-binding fragment thereof. In some embodiments, an acceptable therapeutic dose of the antibody-drug conjugate is 0.1˜30 mg/kg, 0.5˜30 mg/kg, 1˜30 mg/kg, 1˜25 mg/kg, 0.1˜25 mg/kg, 0.1˜20 mg/kg, 1˜20 mg/kg, or 0.5˜20 mg/kg. In some embodiments, a dosing frequency is once every 12 hours, once every day, once every week, once every 2 weeks, once every 4 weeks, once every 5 weeks, once every 6 weeks, once every 7 weeks, once every 8 weeks, once every 9 weeks or once every 10 weeks; or once every month, once every 2 months or once every 3 months, or longer. The therapeutic dose and frequency of administration may vary with the therapeutic regimen.

Various embodiments and preferences of the present disclosure may be combined with each other as long as they are not inherently inconsistent with each other, and the various embodiments formed by the combination are considered to be part of the disclosure of the present application.

The technical solutions of the present disclosure will be more clearly and specifically described below with reference to the embodiments by way of illustration. It should be understood that these embodiments are for illustrative purposes only and are in no way intended to limit the scope of protection of the present disclosure. The scope of protection of the present disclosure is limited only by the claims.

### Examples

The present disclosure is specifically explained by the following examples but is not limited thereto. In addition, these examples are not to be interpretated as limiting in any way. In addition, in the present specification, reagents, solvents, and starting materials that are not particularly described can be easily obtained from commercially available sources.

### Example 1: Preparation of Compound LP-1

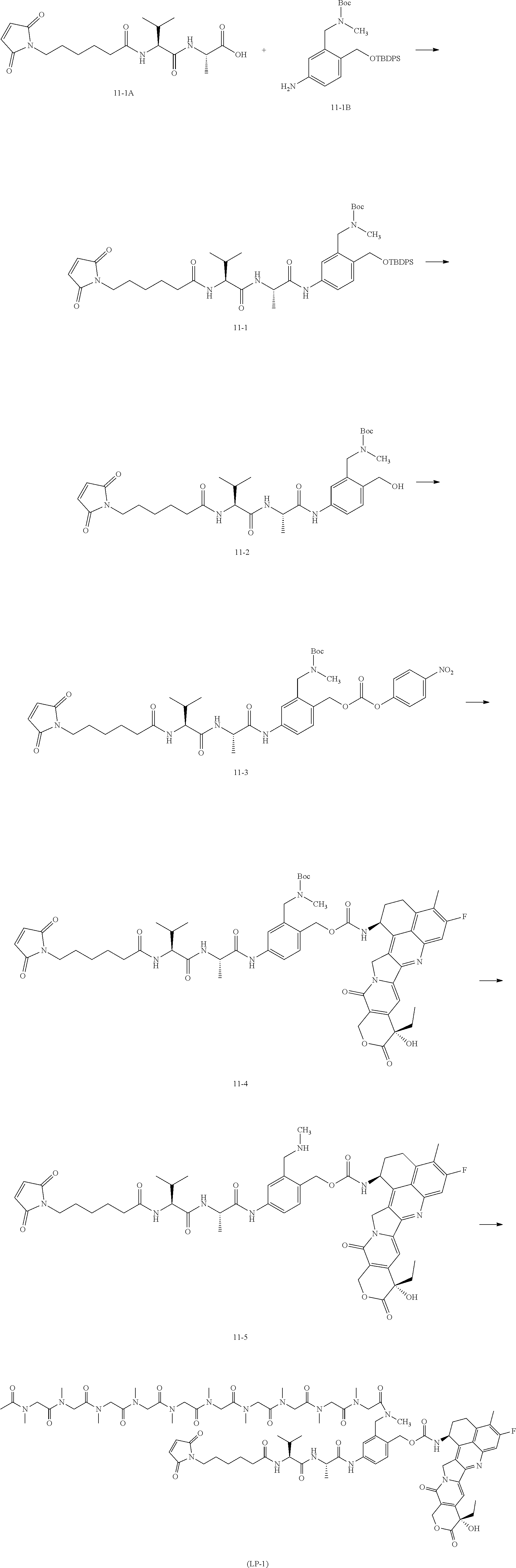

Step 1: Synthesis of intermediate 11-1

DCM (dichloromethane): MeOH (methanol) (v:v=2:1, 90 mL), and EEDQ (2-ethoxy-1-ethoxycarbonyl-1,2-dihydroquinoline) (1.86 g, 7.55 mmol) were added to a mixed solution of compound 11-1A (Mc-Val-Ala-OH, purchased from MedChemExpress Shanghai, 2.4 g, 6.29 mmol) and compound 11-1B (3.18 g, 6.29 mmol) at room temperature. The reaction solution was stirred at room temperature for 24 hours, and solvent therein was removed in vacuo. Then, a crude residue was further purified by flash chromatography to obtain compound 11-1 (3.9 g, 71%). LC-MS (ESI, m/z): 868.49 (M+H).

### Step 2: Synthesis of Intermediate 11-2

Compound 11-1 (2 g, 2.3 mmol) was dissolved in anhydrous THF (tetrahydrofuran) (50 mL), and hydrogen fluoride-pyridine (4.6 g, 46 mmol) was added thereto under an argon atmosphere at 0° C. Then, the reaction mixture was stirred at 0° C. for 2 hours. The reaction was quenched by addition of water. A resultant mixture was extracted with DCM, and the organic phase therein was dried and concentrated. The residue was purified by silica gel chromatography to obtain compound 11-2 (1.1 g, 76%). LC-MS (ESI, m/z): 630.31 (M+H).

### Step 3: Synthesis of Intermediate 11-3

Compound 11-2 (700 mg, 1.11 mmol) was dissolved in anhydrous DMF (N,N-dimethylformamide) (4 mL), and DIPEA (N,N-diisopropylethylamine) (0.39 ml, 2.23 mmol) and 4,4′-dinitrodiphenyl carbonate (406 mg, 1.33 mmol) were added thereto under an argon atmosphere at room temperature. Then, the reaction mixture was stirred at ambient temperature for overnight. Solvent in the reaction mixture was removed by concentration, and the product thus obtained was precipitated using MTBE (tert-butyl ether). A yellow solid was collected by filtration, washed with dietheyl ether, and dried to obtain compound 11-3. LC-MS (ESI, m/z): 795.41 (M+H).

### Step 4: Synthesis of Intermediate 11-4

Compound 11-3 (300 mg, 0.44 mmol) was dissolved in anhydrous DMF (4 mL), and dried pyridine (1 mL) was added thereto, followed by addition of exatecan mesylate (purchased from MedChemExpress Shanghai, 234 mg, 0.44 mmol) and HOBt (1-hydroxybenztriazole) (60 mg, 0.44 mmol). The reaction mixture was stirred at room temperature under argon overnight. The product thus obtained was purified by pre-HPLC to obtain intermediate 11-4 (230 mg, 48%). LC-MS (ESI, m/z): 1091.53 (M+H).

### Step 5: Synthesis of Intermediate 11-5

Compound 11-4 (200 mg, 0.183 mmol) was dissolved in 1 mL anhydrous DCM, and 300 μL TFA (trifluoroacetic acid) was added thereto at 0° C. The reaction mixture was stirred at room temperature for 30 min, and solvent therein was removed by concentration to obtain TFA salt of intermediate 11-5, which may be used in next step without further purification. LC-MS (ESI, m/z): 991.47 (M+H).

### Step 6: Synthesis of Compound LP-1

Compound 11-5 (120 mg, 0.109 mmol) was dissolved in 1 mL anhydrous DMF, and Ac-Sar10-COOH (N-Acetyl Decasarcosine, 84 mg, 0.109 mmol) was added thereto, followed by addition of HATU (2-(7-azabenzotriazole)-N,N,N′,N′-tetramethyluronium hexafluorophosphate, 50 mg, 0.130 mmol) and DIPEA (38 μL, 0.22 mmol). The reaction mixture was stirred at room temperature overnight, and the solvent therein was removed by concentration. The crude product was purified by preparative high-performance liquid chromatography (pre-HPLC) to obtain compound LP-1 (74 mg, 38%). LC-MS (ESI, m/z): 1743.85 (M+H).

### Example 2: Preparation of Compound LP-2

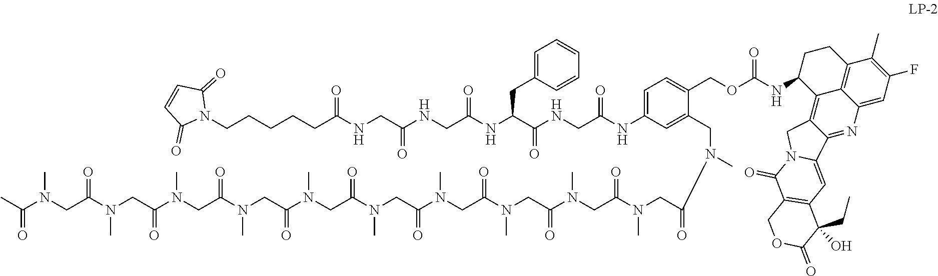 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)CNC(=O)[C@H](Cc2ccccc2)NC(=O)CNC(=O)CNC(=O)CCCCCN2C(=O)C=CC2=O)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)CC3]

The synthesis of compound LP-2 was conducted according to the synthesis steps of compound LP-1. The starting material 11-1A was replaced by Mc-GGFG-OH (purchased from MedChemExpress Shanghai, wherein GGFG represents an amino acid sequence composed of glycine-glycine-phenylalanine-glycine linked by peptide bonds) to obtain compound LP-2 which was beige amorphous solid. LC-MS (ESI, m/z): 1891.90 (M+H).

### Example 3: Preparation of Compound LP-3

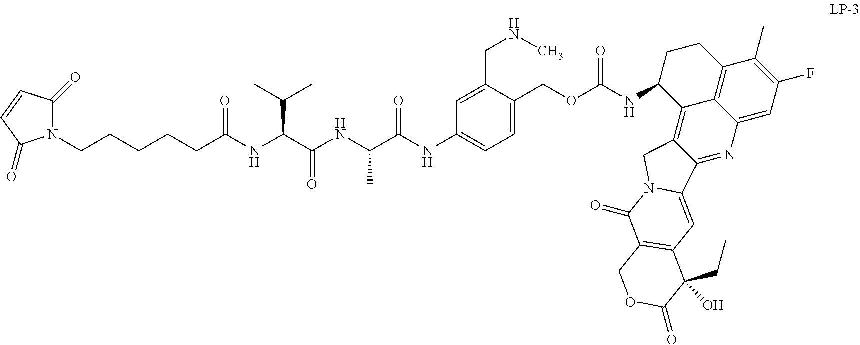 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCCCCN2C(=O)C=CC2=O)C(C)C)cc1CNC)CC3]

Compound LP-3 was intermediate of LP-1. Removing Step 6, intermediate 11-5 would be LP-3.

### Example 4: Detection Method of Antibody-Drug Conjugate

According to the following method, antibody-drug conjugates were concentrated, medium-exchanged, and purified, and concentration of antibodies was measured, and an average number of drug molecules carried by each antibody was calculated, so as to identify antibody-drug conjugates.

### Operation A: Concentration of Antibody or Antibody-Drug Conjugate

An ultrafiltration tube (Amicon Ultra, 50000 MWCO, Millipore Corporation) was taken out, antibody or antibody-drug conjugate solution to be concentrated was add thereto. The ultrafiltration tube was centrifuged until the antibody or antibody-drug conjugate solution therein reached to a required volume, and then taken out.

### Operation B: Measurement of Antibody Concentration

An absorbance of the antibody was measured using a microplate reader (Multiskan GO, Thermo Fisher Scientific) according to the method defined by the manufacturer. The concentration of the antibody is the ratio of the absorption value to the absorption coefficient of the antibody at the detection wavelength.

### Operation C: Medium Exchange of Antibody

According to the instructions provided by the manufacturer (Thermo Fisher Scientific), a Zeba spin desalting column (5 mL, 40K MWCO) was previously equilibrated with phosphate buffered saline (referred to as “PBS7.0/EDTA”, 50 mM, pH 7.0) containing sodium chloride (50 mM) and EDTA (2 mM). 2 mL of sample was loaded on each Zeba spin desalting column, and centrifuged (1000 g, 4 min). Thereafter, a flow-through fraction was collected and concentrated by Operation A, the antibody concentration was determined by Operation B, and the antibody concentration was adjusted with PBS7.0/EDTA.

### Operation D: Purification of Antibody-Drug Conjugate

According to the instructions provided by the manufacturer (Thermo Fisher Scientific), a Zeba spin desalting column (5 mL, 40K MWCO) was previously equilibrated with storage buffer. Histidine-acetate buffer (20 mM histidine, pH 5.5) containing 150 mM NaCl or phosphate buffer (50 mM, pH 7.0) containing 50 mM NaCl was used as storage buffer. The reaction solution containing the antibody-drug conjugate (approximately, 2 mL) was applied to the Zeba spin desalting column, and centrifuged (1000 g, 4 min). Thereafter, a flow-through fraction (approximately, 2 mL) was collected, and an elution process was repeated twice to remove the unbound linker-payload and low molecular weight compounds including reducing agents.

### Operation E: Measurement of Concentration of Antibody in the Antibody-Drug Conjugate and Average Number of Drug Molecules Connected to Each Antibody (DAR Value)-(1)

The concentration of the drug conjugated on the antibody-drug conjugate can be obtained by measuring the UV absorption values of the antibody-drug conjugate aqueous solution at 280 nm and 370 nm and calculating by the following formula.

At any given wavelength, a total absorbance of a system is equal to the sum of the absorbances of all light-absorbing chemicals present in the system (additivity of absorbance). Therefore, assuming that the molar absorption coefficient of the antibody and the drug remains unchanged before and after conjugation of the antibody and the drug, the concentration of antibody and that of the drug in the antibody-drug conjugate can be expressed by the following formulas.

$${A}_{280} = {A}_{D , 280} + {A}_{A , 280} = {ε}_{D , 280} {C}_{D} + {ε}_{A , 280} {C}_{A} \qquad Equation ( 1 )$$

$${A}_{370} = {A}_{D , 370} + {A}_{A , 370} = {ε}_{D , 370} , {C}_{D} + {ε}_{A , 370} {C}_{A} \qquad Equation ( 2 )$$

A₂₈₀ represents a total absorption value of the antibody-drug conjugate aqueous solution at 280 nm, A₃₇₀ represents a total absorption value of the antibody-drug conjugate aqueous solution at 370 nm. A_{A,280} represents a absorption value of the antibody at 280 nm, A_{A,370} represents a absorption value of the antibody at 370 nm, A_{D,280} represents a absorption value of the conjugate precursors (drug) at 280 nm, A_{D,370} represents a absorption value of the conjugate precursors at 370 nm, ε_{A,280} represents a molar extinction coefficient of the antibody at 280 nm, ε_{A,370} represents a molar extinction coefficient of the antibody at 370 nm, ε_{D,280} represents a molar extinction coefficient of the conjugate precursors at 280 nm, ε_{D,370} represents a molar extinction coefficient of the conjugate precursors at 370 nm, C_{A} represents concentration of antibody in antibody-drug conjugate, and C_{D} represents concentration of drug molecule in antibody-drug conjugate.

In this case, ε_{A,280}, ε_{A,370}, ε_{D,280}, and ε_{D,370} are all known values (calculated from the sequence of the antibody or measured by the UV absorption of the compound). For example, ε_{A,280} can be calculated from the amino acid sequence of the antibody using a known method (Protein Science, 1995, Vol. 4, pp. 2411-2423). The antibody usually has no absorbance at 370 nm, and thus ε_{A,370} is usually 0. The values of ε_{D,280} and ε_{D,370} can be calculated by measuring the change of absorbance of conjugate precursors with concentration at 280 nm and 370 nm, respectively, and using Lambert-Beer law (absorbance=molar concentration×molar extinction coefficient×optical path). C_{A} and C_{D} can be obtained by measuring the absorbance values A₂₈₀ and A₃₇₀ of antibody-drug conjugates at 280 nm and 370 nm, and then solving the simultaneous (1) and (2). In addition, the average number of drug molecules connected to each antibody (DAR value) can be obtained by dividing C_{D} by C_{A}.

### Operation F: Average Number of Drug Molecules Connected to Each Antibody (DAR Value)-(2)

The average number of drug molecules conjugated to each antibody molecule in the antibody-drug conjugate can be determined by liquid chromatography-mass spectrometry (LC-MS) analysis method in addition to the aforementioned “operation E”, which is explained as follows.

Preparation of sample for high performance liquid chromatography-mass spectrometry (LC-MS) analysis (reduction of antibody-drug conjugate) 3 μL 100 mM of a reducing agent, dithiothreitol (DTT) and 21 μL of deionized water were added to the antibody-drug conjugate (about 5 mg/mL, 6 μL). The reaction solution was incubated in a water bath of 37° C. for 30 minutes, and disulfide bonds between the light and heavy chains and between the heavy chains in the antibody-drug conjugate were completely cleaved, and a resultant sample could be used for LC-MS detection and analysis.

### High Performance Liquid Chromatography Parameters

Column type: Agilent PLRP-S, 1000 Å, 50×2.1 mm, 8 μm.
Detection wavelength: 280 nm
Band width: 4 nm
Column temperature: 80° C.
Autosampler thermostat: 5° C.
Flow rate: 0.5 mL/min
Injection volume: 5 μL
Mobile phase A: 0.05% TFA, H₂O
Mobile phase B: 0.05% TFA, ACN
Elution program: (B %: 25%-34% (0-0.7 minutes), 34%-45% (0.7-5 minutes), 45%-90% (5-6 minutes), 90% (6-7 minutes), 90%-25% (7-7.10 minutes), 25% (7.10-10 minutes)

### MS Parameters

Atomization gas temperature: 350° C.
Atomization gas flow rate: 13 L/min
Atomizer: 45 psig
Capillary voltage: 5000 V
Fragmentation voltage: 350 V
Mass-to-charge ratio range: 500-8000 m/z
Acquisition rate: 1 spectra/s

### Data Analysis

A light chain conjugated to i drug molecules is represented as Li, and a heavy chain conjugated to i small molecules is represented as Hi, which can be determined according to ESI scan of the mass spectrometer.

A corrected peak area ratio can be obtained by substituting the peak area percentage (%) of each chain into the following formula.

$$\begin{matrix}Peak area ratio of light chain conjugated to i drug molecules = 100 \% \times {A}_{Li} / ( {A}_{L 0} + {A}_{L 1} ) \\ Peak area ratio of heavy chain conjugated to i drug molecules = 100 \% \times {A}_{Hi} / ( {A}_{H 0} + {A}_{H 1} + {A}_{H 2} + {A}_{H 3} )\end{matrix}$$

Average number of drug molecules conjugated to each antibody can be calculated by the following formula.

$$Average number of conjugated drug molecules = ( L 0 peak area ratio \times 0 + L 1 peak area ratio \times 1 + H 0 peak area ratio \times 0 + H 1 peak area ratio \times 1 + H 2 peak area ratio \times 2 + H 3 peak area ratio \times 3 ) \times 2$$

### Operation G: Average Number of Drug Molecules Connected to Each Antibody (DAR Value)-(3)

In addition to the aforementioned “Operation E” and “Operation F”, the average number of drug molecules conjugated to each antibody molecule in the antibody-drug conjugate may also be determined using the hydrophobic interaction chromatography (HIC) analysis method described below.

The elution of antibody-drug conjugate on hydrophobic interaction chromatography column was based on differences in concentration of salt ions in the eluent—as the concentration of salt ions decreases, the number of small molecule drugs in the eluted antibody-drug conjugates increases, that is, antibody-drug conjugate with low DAR value was preferentially eluted. The peak sequence of each component was D0 (antibody unconjugated to any linker-payload), D2 (antibody conjugated to 2 linkers-payloads), D4 (antibody conjugated to 4 linkers-payloads), D6 (antibody conjugated to 6 linkers-payloads), D8 (antibody conjugated to 8 linkers-payloads). The percentage content of each component can be obtained by measuring the peak area ratio of each peak. Then the HIC-DAR of corresponding samples are calculated as follows:

$$Average number of conjugated drug molecules = D 0 peak area ratio \times 0 + D 2 peak area ratio \times 2 + D 4 peak area ratio \times 4 + D 6 peak area ratio \times 6 + D 8 peak area ratio \times 8$$

### Operation H: Measurement of Aggregates in Antibody-Drug Conjugate

Aggregates in the antibody-drug conjugate were detected using size exclusion chromatography in high performance liquid chromatography. The method was as follows.
High performance liquid chromatography system: Agilent 1260 Infinity II HPLC system
Detector: Ultraviolet absorption spectrometer (Detection wavelength: 280 nm)
Column type: TOSOH TSKgel G3000SWXL (7.8×300 mm, 5 μm)
Mobile phase: 200 mmol/L KHPO₄, 150 mmol/L NaCl, 15% (v/v) isopropyl alcohol, pH7.0
Flow rate: 0.75 mL/min
Analysis time: 18 min
Column temperature: Room temperature
Injection volume: 50 μg

### Data Analysis:

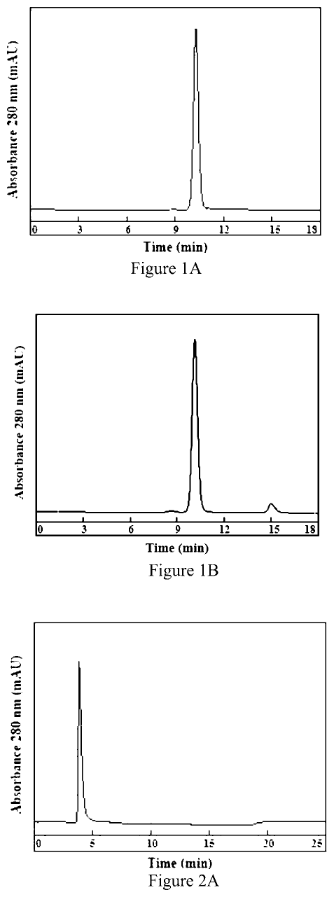

A size exclusion chromatogram of a quality control material (QC, 02-1 naked antibody, i.e., antibody numbered 02-1 which was not conjugated to a linker-payload) was shown in FIG. 1A; a size exclusion chromatogram of a quality control material (QC, Hu1H2-2 naked antibody, i.e., the antibody numbered Hu1H2-2 which was not conjugated to linker-payload) was shown in FIG. 1B, and an aggregate content of Hu1H2-2 naked antibody was 1.17%. A retention time of the main peak (single peak) of quality control material of 150 kDa was between 9.5 and 10.5 minutes. The retention time of the aggregates should be earlier than that of the above-mentioned monomers.

### Operation I: Comparison of Hydrophobicity of Antibody-Drug Conjugates

The hydrophobicity of antibody-drug conjugates was analyzed using high performance liquid chromatography hydrophobic interaction chromatography (HIC). The method was follows:
High performance liquid chromatography system: Agilent 1260 Infinity II HPLC system
Detector: Ultraviolet absorption spectrometer (Detection wavelength: 280 nm)
Column type: TOSOH TSKgel Butyl-NPR (4.6 mm I.D.×3.5 cm, 2.5 μm)
Mobile phase A: mol/L (NH₄)₂SO₄, 50 mmol/L KHPO₄, pH7.0
Mobile phase B: 50 mmol/L KHPO₄, 25% (v/v) isopropanol, pH7.0
Analysis time: 25 min
Column temperature: Room temperature
Elution procedure (B %): 0%-25% (0-1 minutes), 25% (1-3 minutes), 25%-80% (3-13 minutes), 80% (13-17 minutes), 80%-0% (17-17.10 minutes), 0% (17.10-25 minutes)
Injection volume: 10 μL

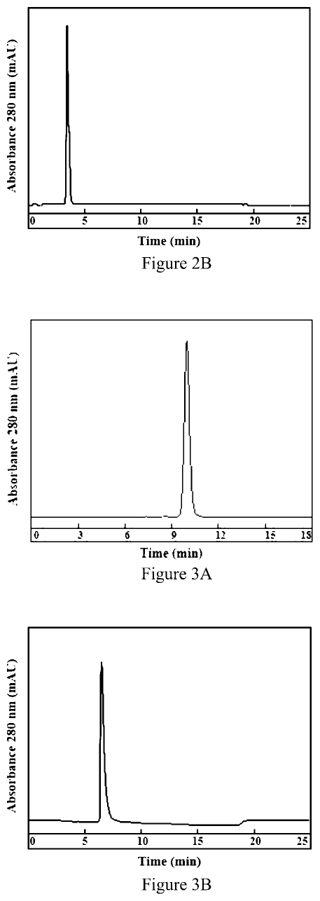

Data analysis: A hydrophobic interaction chromatography chromatogram of a quality control material (QC, 02-1 naked antibody) was shown in FIG. 2A. A hydrophobic chromatogram of a quality control material (QC, Hu1H2-2 naked antibody) was shown in FIG. 2B, and a retention time thereof was 3.387 minutes. The sample with a shorter retention time was less hydrophobic. The antibody-drug conjugate was more hydrophobic than the unconjugated naked antibody, thus a retention time thereof was longer.

### Example 5: Preparation of Antibody-Drug Conjugate 02-1-LP1 (DAR 8)

Reduction of the antibody: a medium of the antibody was exchanged with PBS7.0/EDTA by operation B (an extinction coefficient of the antibody at 280 nm was 1.523 mL mg⁻¹ cm⁻¹) and operation C in Example 4, and a concentration of the antibody after medium exchange was 23.2 mg/mL. 0.482 mL of 5 mM TCEP solution (corresponding to 7 times equivalent of the content of the antibody) was added to 2.155 mL of 02-1 antibody aqueous solution, and 4.31 mL of 50 mM phosphate buffer (pH7.0, PBS7.0) and 69.47 μL of 200 mM EDTA solution were added thereto at the same time. After confirming that the pH of the solution was 7.0±0.1, the mixed solution was placed in an environment of 37° C. to react for 2 hours.

VH sequence of 02-1 antibody was the amino acid sequence shown in SEQ ID NO: 84, VL sequence thereof was the amino acid sequence shown in SEQ ID NO: 85. For the purpose of preparation and/or detection in the examples, the constant region sequence of human IgG1 was used as the constant region of 02-1 antibody, the amino acid sequence shown in SEQ ID NO: 102 was selected as the heavy chain constant region sequence of 02-1 antibody; and the amino acid sequence shown in SEQ ID NO: 103 was selected as the light chain constant region sequence of 02-1 antibody.

Conjugation of antibody and linker-payload: the above mixed solution was incubated at 4° C. for 10 minutes. The linker-payload LP-1 prepared in Example 1 was dissolved in N,N-dimethylacetamide (DMA), and 0.489 mL (corresponding to 14.2 times equivalent of the content of the antibody) of the solution was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate 02-1-LP1.

Characterization of the antibody-drug conjugate: the obtained antibody-drug conjugate was characterized using operation E (ε_{D,280}=6384 and ε_{D,370}=16180), operation F, operation H, and operation I in Example 4.

The concentration of the antibody-drug conjugate measured and calculated by operation E was 12.13 mg/mL, and an average number of payloads conjugated per antibody measured and calculated by operation E was 7.78. The average number of payloads conjugated per antibody measured by operation F was 7.80. FIG. 3A showed a detection graph of aggregates, and a content of the aggregates in the antibody-drug conjugate 02-1-LP1 measured by operation H was 1.18%. FIG. 3B showed a detection graph of the hydrophobic interaction chromatography of the antibody-drug conjugate 02-1-LP1, and a retention time of the antibody-drug conjugate 02-1-LP1 measured by operation I was 6.499 min.

### Example 6: Preparation of Antibody-Drug Conjugate Hu1H2-2-LP1 (DAR 8)

Reduction of the antibody: a medium of the antibody was exchanged with PBS7.0/EDTA by operation B (an extinction coefficient of the antibody at 280 nm was 1.54 mLmg⁻¹ cm⁻¹) and operation C in Example 4, and a concentration of the antibody after the medium exchange was 9.37 mg/mL. 80 μL of 5 mM TCEP solution (corresponding to 10 times equivalent of the content of the antibody) was added to 640.34 μL of Hu1H2-2 (HIS1H2-2) antibody aqueous solution, and 0.2 mL of 50 mM PBS7.0 and 79.66 μL of deionized water were added thereto at the same time. After confirming that the pH of the solution was 7.0±0.1, the mixture was placed in an environment of 37° C. to react for 2 hours.

VH sequence of Hu1H2-2 antibody was the amino acid sequence shown in SEQ ID NO: 100, VL sequence thereof was the amino acid sequence shown in SEQ ID NO: 104. For the purpose of preparation and/or detection in the examples, the amino acid sequence shown in SEQ ID NO: 102 was selected as the heavy chain constant region of Hu1H2-2 antibody; and the amino acid sequence shown in SEQ ID NO: 103 was selected as the light chain constant region of Hu1H2-2 antibody.

Conjugation of antibody and linker-payload: the above mixed solution was incubated at 4° C. for 10 minutes. The linker-payload LP-1 prepared in Example 1 was dissolved in DMA, and 72 μL (corresponding to 18 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Hu1H2-2-LP1.

Characterization of the antibody-drug conjugate: the obtained antibody-drug conjugate was characterized using operation E (ε_{D,280}=6384 and ε_{D,370}=16180), operation H, and operation I in Example 4.

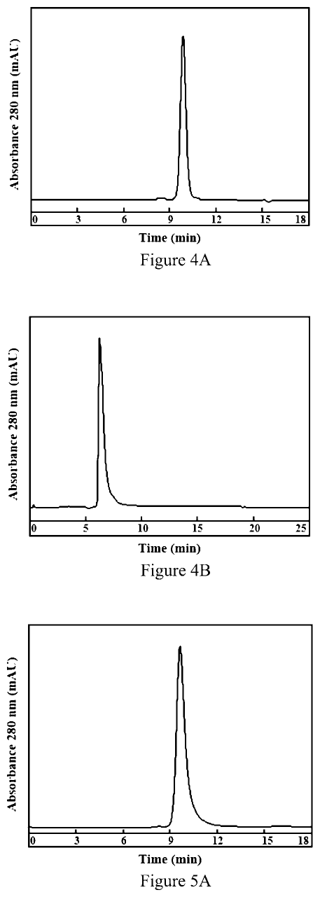

The concentration of the antibody-drug conjugate measured and calculated by operation E was 6.74 mg/mL, and an average number of payloads conjugated per antibody measured and calculated by operation E was 9.20. FIG. 4A showed a detection graph of aggregates, and a content of the aggregates in the antibody-drug conjugate Hu1H2-2-LP1 measured by operation H was 1.24%. FIG. 4B showed a detection graph of the hydrophobic interaction chromatography of the antibody-drug conjugate Hu1H2-2-LP1, and a retention time of the antibody-drug conjugate Hu1H2-2-LP1 measured by operation I was 6.239 min.

### Example 7: Preparation of Antibody-Drug Conjugate Hu1H2-2-LP1 (DAR 4)

Reduction of the antibody: a medium of the antibody was exchanged with PBS7.0/EDTA by operation B (an extinction coefficient of the antibody at 280 nm was 1.54 mL mg⁻¹ cm⁻¹) and operation C in Example 4, and a concentration of the antibody after medium exchange was 23 mg/mL. 23.52 μL of 5 mM TCEP solution was added to 456.52 μL of Hu1H2-2 antibody aqueous solution, and 0.28 mL of 50 mM PBS7.0 and 639.96 μL of deionized water were added thereto at the same time. After confirming that the pH of the solution was 7.0±0.1, the mixture was placed in an environment of 37° C. to react for 2 hours.

Conjugation of antibody and linker-payload: the above mixed solution was incubated at 4° C. for 10 minutes. The linker-payload LP-1 prepared in Example 1 was dissolved in DMA, and 112 μL (corresponding to 8 times equivalent of the content of the antibody) of the solution then was added to the mixed solution. The reaction of the mixed solution continued at 22° C. for 30 minutes.

Characterization of the antibody-drug conjugate: the obtained antibody-drug conjugate was characterized using operation E (ε_{D,280}=6384 and ε_{D,370}=16180) and operation H in Example 4.

For the prepared antibody-drug conjugate Hu1H2-2-LP1 (DAR4), the concentration of the antibody-drug conjugate measured and calculated by operation E was 6.14 mg/mL, and an average number of payloads conjugated per antibody measured and calculated by operation E was 3.59. FIG. 5A showed a detection graph of aggregates, and a content of the aggregates in the antibody-drug conjugate Hu1H2-2-LP1 (DAR4) measured by operation H was 0.44%. FIG. 5B showed a detection graph of the hydrophobic interaction chromatography of the antibody-drug conjugate Hu1H2-2-LP1.

### Example 8: Preparation of Antibody-Drug Conjugate Hu1H2-2-LP2 (DAR 8)

Reduction of the antibody: a reduction method in Example 8 is the same as that of the antibody in Example 6. The amino acid sequence of Hu1H2-2 antibody in Example 8 is the same as that of the Hu1H2-2 antibody in Example 6.

Conjugation of antibody and linker-payload: the mixed solution in the reduction step of the antibody was incubated at an environment of 4° C. for 10 minutes. The linker-payload LP-2 prepared in Example 2 was dissolved in DMA, and 72 μL (corresponding to 18 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Hu1H2-2-LP2.

Characterization of the antibody-drug conjugate: the obtained antibody-drug conjugate was characterized using the above operation E (ε_{D,280}=5814 and ε_{D,370}=14742), operation F, operation H, and operation I.

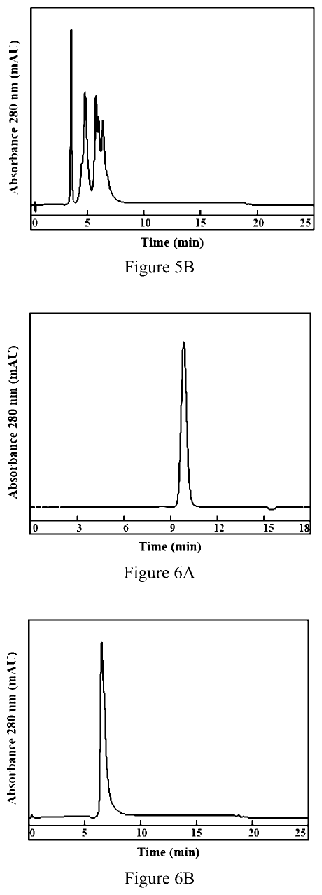

The concentration of the antibody-drug conjugate measured and calculated by operation E was 6.64 mg/mL, and an average number of payloads conjugated per antibody measured and calculated by operation E was 9.92. FIG. 6A showed a detection graph of aggregates, and a content of the aggregates in the antibody-drug conjugate Hu1H2-2-LP2 measured by operation H was 1.37%. FIG. 6B showed a detection graph of the hydrophobic interaction chromatography of the antibody-drug conjugate Hu1H2-2-LP2, and a retention time of the antibody-drug conjugate Hu1H2-2-LP2 measured by operation I was 6.561 min.

### Example 9: Preparation of Antibody-Drug Conjugate Hu1H2-2-LP3 (DAR 8)

Reduction of the antibody: a medium of the antibody was exchanged with PBS7.0/EDTA by operation B (an extinction coefficient of the antibody at 280 nm was 1.54 mLmg⁻¹ cm⁻¹) and operation C in Example 4, and a concentration of the antibody after medium exchange was 23.07 mg/mL. 70 μL of 5 mM TCEP solution (corresponding to 7 times equivalent of the content of the antibody) was added to 325.10 μL of Hu1H2-2 antibody aqueous solution, and 0.3 mL of 50 mM PBS7.0 and 804.90 μL of deionized water were added thereto at the same time. After confirming that the pH of the solution was 7.0±0.1, the mixture was placed in an environment of 37° C. to react for 2 hours.

The amino acid sequence of Hu1H2-2 antibody is the same as that of Hu1H2-2 antibody in Example 6.

Conjugation of antibody and linker-payload: the above mixed solution was incubated at 4° C. for 10 minutes. The linker-payload LP-3 prepared in Example 3 was dissolved in DMA, and 90 μL (corresponding to 18 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D to finally obtain antibody-drug conjugate Hu1H2-2-LP3.

Characterization of the antibody-drug conjugate: the obtained antibody-drug conjugate was characterized using the above operation E (ε_{D,280}=5186 and ε_{D,370}=13688), operation F, operation H, and operation I.

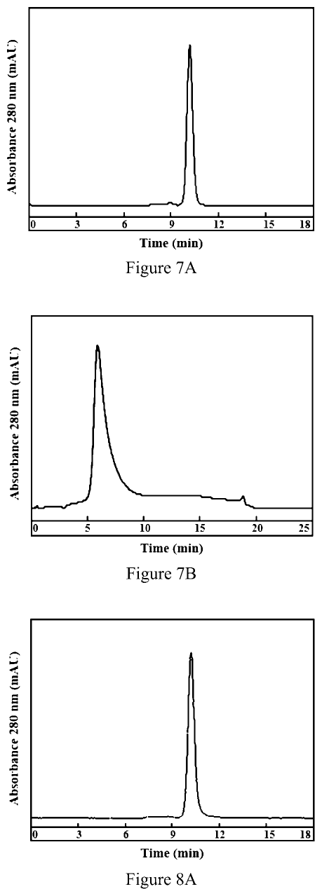

The concentration of the antibody-drug conjugate measured and calculated by operation E was 4.10 mg/mL, and an average number of payloads conjugated per antibody measured and calculated by operation E was 8.50. FIG. 7A showed a detection graph of aggregates, and a content of the aggregates in the antibody-drug conjugate Hu1H2-2-LP3 measured by operation H was 3.37%. FIG. 7B showed a detection graph of the hydrophobic interaction chromatography of the antibody-drug conjugate Hu1H2-2-LP3, and a retention time of the antibody-drug conjugate Hu1H2-2-LP3 measured by operation I was 5.878 min.

### Comparative Example 1: Preparation of Antibody-Drug Conjugate 02-1-Vc-MMAE (DAR 4)

Reduction of the antibody: a medium of the antibody was exchanged with PBS7.0/EDTA by operation B (an extinction coefficient of the antibody at 280 nm was 1.523 mLmg⁻¹ cm⁻¹) and operation C in Example 4, and a concentration of the antibody after medium exchange was 31.6 mg/mL. 0.076 mL of 5 mM TCEP solution (corresponding to 2.3 times equivalent of the content of the antibody) was added to 0.759 mL of 02-1 antibody aqueous solution, and 0.48 mL of 50 mM PBS7.0 and 1.084 mL of deionized water were added thereto at the same time. After confirming that the pH of the solution was 7.0±0.1, the mixture was placed in an environment of 37° C. to react for 2 hours. The sequence of 02-1 antibody was the same as that of 02-1 antibody in Example 5.

Conjugation of antibody and linker-payload: the above mixed solution was incubated at 4° C. for 10 minutes. The linker-payload vc-MMAE (DC Chemicals, DC7556) was dissolved in DMA, 0.322 mL (corresponding to 7 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate 02-1-vc-MMAE.

Characterization of the antibody-drug conjugate: the obtained antibody-drug conjugate was characterized using operation B, operation G, operation H and operation I in Example 4.

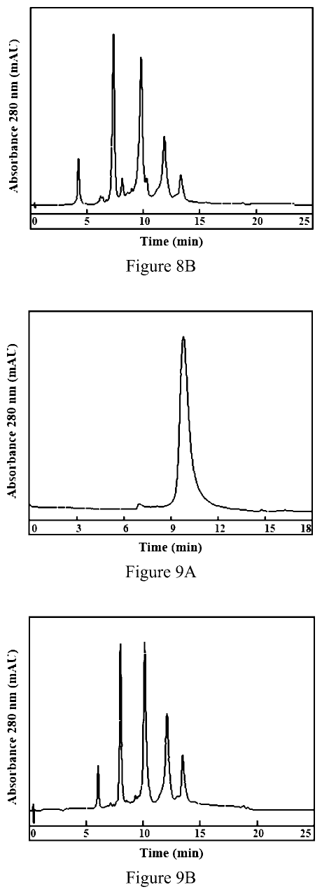

The concentration of antibody-drug conjugate calculated by operation B measurement was 9.71 mg/mL. FIG. 8A showed a detection graph of aggregates, and a content of the aggregates in the antibody-drug conjugate 02-1-vc-MMAE measured by operation H was 2.74%. FIG. 8B showed a detection graph of the hydrophobic interaction chromatography of the antibody-drug conjugate 02-1-vc-MMAE. An average number of payloads conjugated per antibody in the antibody-drug conjugate 02-1-vc-MMAE measured and calculated by operation G and operation I was 3.99 (HIC-DAR).

### Comparative Example 2: Preparation of Antibody-Drug Conjugate Rituximab-Vc-MMAE (DAR 4)

Reduction of the antibody: a medium of the antibody was exchanged with PBS7.0/EDTA by operation B (an extinction coefficient of the antibody at 280 nm was 1.5 mLmg⁻¹ cm⁻¹) and operation C in Example 4, and a concentration of the antibody after medium exchange was 12 mg/mL. 0.015 mL of 5 mM TCEP solution (corresponding to 2.2 times equivalent of the content of the antibody) was added to 0.438 mL aqueous solution of Rituximab antibody (purchased from Shanghai Minbiotech Co., Ltd.), and 0.14 mL of 50 mM PBS7.0 and 0.107 mL of deionized water were added thereto at the same time. After confirming that the pH of the solution was 7.0±0.1, the mixture was placed in an environment of 37° C. to react for 2 hours.

Conjugation of antibody and linker-payload: the above mixed solution was incubated at 4° C. for 10 minutes. The linker-payload vc-MMAE was dissolved in DMA, 0.049 mL (corresponding to 7 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Rituximab-vc-MMAE.

Characterization of the antibody-drug conjugate: the obtained antibody-drug conjugate was characterized using operation B, operation G, operation H, and operation I in Example 4.

The concentration of the antibody-drug conjugate measured and calculated by operation B was 3.78 mg/mL. FIG. 9A showed a detection graph of aggregates, and a content of the aggregates in the antibody-drug conjugate Rituximab-vc-MMAE measured by operation H was 3.78%. FIG. 9B showed a detection graph of the hydrophobic interaction chromatography of the antibody-drug conjugate Rituximab-vc-MMAE, and an average number of payloads conjugated per antibody in the antibody-drug conjugate Rituximab-vc-MMAE measured and calculated by operation G and operation I was 4.39 (HIC-DAR).

### Comparative Example 3: Preparation of Antibody-Drug Conjugate Rituximab-LP1 (DAR 8)

Reduction of the antibody: a medium of the antibody was exchanged with PBS7.0/EDTA by operation B (an extinction coefficient of the antibody at 280 nm was 1.5 mLmg⁻¹ cm⁻¹) and operation C in Example 4, and a concentration of the antibody after medium exchange was 11.8 mg/mL. 0.667 mL of 5 mM TCEP solution (corresponding to 7 times equivalent of the content of the antibody) was added to 6.05 mL of Rituximab antibody aqueous solution, and 1.02 mL of 100 mM PBS7.0 and 2.46 mL of deionized water were added thereto at the same time. After confirming that the pH of the solution was 7.0±0.1, the mixture was placed in an environment of 37° C. to react for 2 hours.

Conjugation of antibody and linker-payload: the above mixed solution was incubated at 4° C. for 10 minutes. The linker-payload LP-1 was dissolved in DMA, and 0.667 mL (corresponding to 14 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Rituximab-LP1.

Characterization of the antibody-drug conjugate: the obtained antibody-drug conjugate was characterized using operation E (ε_{D,280}=6384 and ε_{D,370}=16180), operation H, and operation I in Example 4.

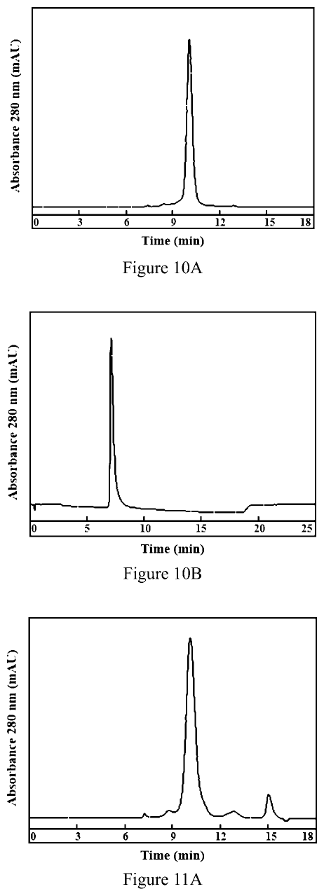

The concentration of the antibody-drug conjugate measured and calculated by operation E was 6.23 mg/mL, and an average number of payloads conjugated per antibody measured and calculated by operation E was 7.30. FIG. 10A showed a detection graph of aggregates, and a content of the aggregates in the antibody-drug conjugate Rituximab-LP1 measured by operation H was 4.70%. FIG. 10B showed a detection graph of the hydrophobic interaction chromatography of the antibody-drug conjugate Rituximab-LP1, and a retention time of the antibody-drug conjugate Rituximab-LP1 measured by operation I was 7.094 min.

### Comparative Example 4: Preparation of Antibody-Drug Conjugate Human IgG-GGFG-DXd (DAR 8)

Reduction of the antibody: a medium of the antibody was exchanged with PBS7.0/EDTA by operation B (an extinction coefficient of the antibody at 280 nm was 1.35 mLmg⁻¹ cm⁻¹) and operation C in Example 4, and a concentration of the antibody after medium exchange was 10 mg/mL. 0.327 mL of 5 mM TCEP solution (corresponding to 10 times equivalent of the content of the antibody) was added to 2.45 mL of human IgG protein aqueous solution (purchased from Beijing Solarbio Science & Technology Co., Ltd., product No. SP001), and 0.7 mL of 50 mM PBS7.0 and 23 μL of deionized water were added thereto at the same time. After confirming that the pH of the solution was 7.0±0.1, the mixture was placed in an environment of 37° C. to react for 2 hours.

Conjugation of antibody and linker-payload: the above mixed solution was incubated at 4° C. for 10 minutes. The linker-payload GGFG-DXd (purchased from DC Chemicals, DC50025) was dissolved in DMA, and 0.294 mL (corresponding to 18 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Human IgG-GGFG-DXd (referred to as “Human IgG-DXD”). Human IgG and HuIgG are used interchangeably in the disclosure.

Characterization of the antibody-drug conjugate: the obtained antibody-drug conjugate was characterized using operation E (ε_{D,280}=5178 and ε_{D,370}=20217), operation H, and operation I in Example 4.

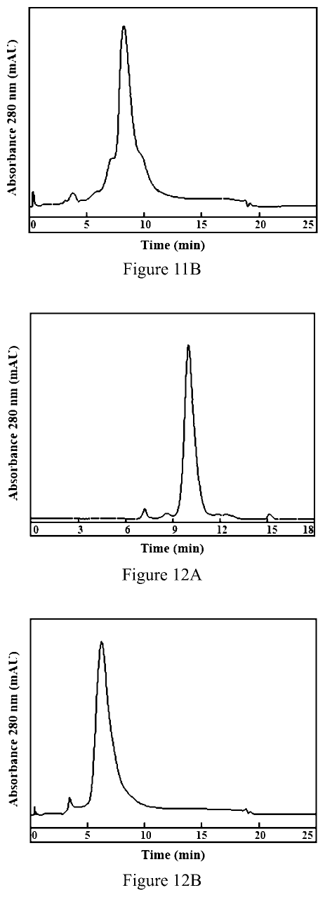

The concentration of the antibody-drug conjugate measured and calculated by operation E was 8.20 mg/mL, and an average number of payloads conjugated per antibody measured and calculated by operation E was 7.08. FIG. 11A showed a detection graph of aggregates, and a content of the aggregates in the antibody-drug conjugate Human IgG-DXD measured by operation H was 4.25%. FIG. 11B showed a detection graph of the hydrophobic interaction chromatography of the antibody-drug conjugate Human IgG-DXD, and a retention time of the antibody-drug conjugate Human IgG-DXD measured by operation I was 8.222 min.

### Comparative Example 5: Preparation of Antibody-Drug Conjugate Human IgG-LP1 (DAR 8)

Reduction of the antibody: a medium of the antibody was exchanged with PBS7.0/EDTA by operation B (an extinction coefficient of the antibody at 280 nm was 1.35 mLmg⁻¹ cm⁻¹) and operation C in Example 4, and a concentration of the antibody after medium exchange was 10 mg/mL. 0.267 mL of 5 mM TCEP solution (corresponding to 10 times equivalent of the content of the antibody) was added to 1.33 mL of human IgG protein aqueous solution (purchased from Beijing Solarbio Science & Technology Co., Ltd. product No. SP001), and 0.4 mL of 50 mM PBS7.0 was added thereto at the same time. After confirming that the pH of the solution was 7.0±0.1, the mixture was placed in an environment of 37° C. to react for 2 hours.

Conjugation of antibody and linker-payload: the above mixed solution was incubated at 4° C. for 10 minutes. The linker-payload LP-1 was dissolved in DMA, and 0.2 mL (corresponding to 15 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Human IgG-LP1.

Characterization of the antibody-drug conjugate: the obtained antibody-drug conjugate was characterized using operation E (ε_{D,280}=6384 and ε_{D,370}=16180), operation H, and operation I in Example 4.

The concentration of the antibody-drug conjugate measured and calculated by operation E was 11.08 mg/mL, and an average number of payloads conjugated per antibody measured and calculated by operation E was 9.15. FIG. 12A showed a detection graph of aggregates, and a content of the aggregates in the antibody-drug conjugate Human IgG-LP1 measured by operation H was 4.38%. FIG. 12B showed a detection graph of the hydrophobic interaction chromatography of the antibody-drug conjugate Human IgG-LP1, and a retention time of the antibody-drug conjugate Human IgG-LP1 measured by operation I was 6.213 min.

### Comparative Example 6: Preparation of Antibody-Drug Conjugate Human IgG-LP1 (DAR 4)

Reduction of the antibody: a medium of the antibody was exchanged with PBS7.0/EDTA by operation B (an extinction coefficient of the antibody at 280 nm was 1.35 mLmg⁻¹ cm⁻¹) and operation C in Example 4, and a concentration of the antibody after medium exchange was 15 mg/mL. 34.02 μL of 5 mM TCEP solution (corresponding to 2.43 times equivalent of the content of the antibody) was added to 0.7 mL of human IgG protein aqueous solution (purchased from Beijing Solarbio Science & Technology Co., Ltd., product No. SP001), and 0.28 mL of 50 mM PBS7.0 was added thereto at the same time. After confirming that the pH of the solution was 7.0±0.1, the mixture was placed in an environment of 37° C. to react for 2 hours.

Conjugation of antibody and linker-payload: the above mixed solution was incubated at 4° C. for 10 minutes. The linker-payload LP-1 was dissolved in DMA, and 0.112 mL (corresponding to 8 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Human IgG-LP1 (DAR4).

Characterization of the antibody-drug conjugate: the obtained antibody-drug conjugate was characterized using operation E (ε_{D,280}=6384 and ε_{D,370}=16180), operation H, and operation I in Example 4.

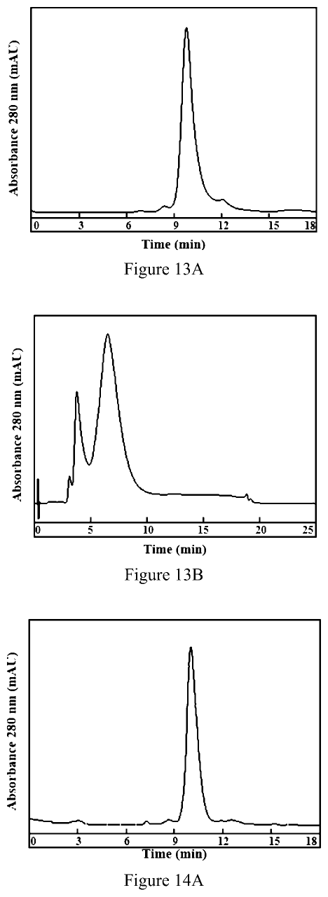

The concentration of the antibody-drug conjugate measured and calculated by operation E was 5.75 mg/mL, and an average number of payloads conjugated per antibody measured and calculated by operation E was 3.53. FIG. 13A showed a detection graph of aggregates, and a content of the aggregates in the antibody-drug conjugate Human IgG-LP1 (DAR4) measured by operation H was 1.90%. FIG. 13B showed a detection graph of the hydrophobic interaction chromatography of the antibody-drug conjugate Human IgG-LP1.

### Comparative Example 7: Preparation of Antibody-Drug Conjugate Human IgG-LP2 (DAR 8)

Reduction of the antibody: a medium of the antibody was exchanged with PBS7.0/EDTA by operation B (an extinction coefficient of the antibody at 280 nm was 1.35 mLmg⁻¹ cm⁻¹) and operation C in Example 4, and a concentration of the antibody after medium exchange was 10 mg/mL. 0.093 mL of 5 mM TCEP solution (corresponding to 10 times equivalent of the content of the antibody) was added to 0.467 mL of human IgG protein aqueous solution (purchased from Beijing Solarbio Science & Technology Co.Ltd., product No. SP001), and 0.2 mL of 50 mM PBS7.0 and 0.24 mL of deionized water were added thereto at the same time. After confirming that the pH of the solution was 7.0±0.1, the mixture was placed in an environment of 37° C. to react for 2 hours.

Conjugation of antibody and linker-payload: the above mixed solution was incubated at 4° C. for 10 minutes. The linker-payload LP-2 was dissolved in DMA, and 0.07 mL (corresponding to 15 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Human IgG-LP2.

Characterization of the antibody-drug conjugate: the obtained antibody-drug conjugate was characterized using operation E (ε_{D,280}=5814 and ε_{D,370}=14742), operation H, and operation I in Example 4.

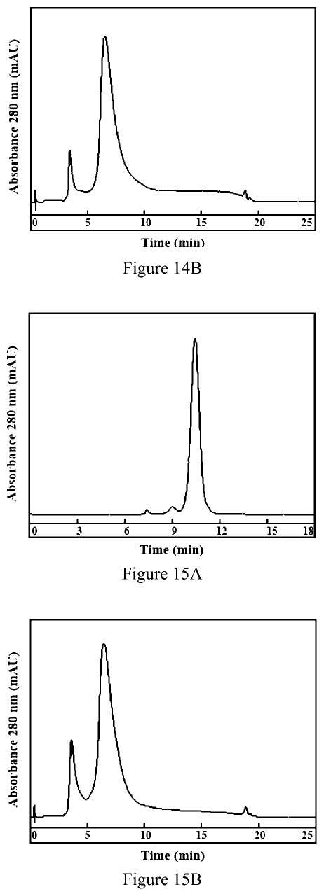

The concentration of the antibody-drug conjugate measured and calculated by operation E was 4.68 mg/mL, and an average number of payloads conjugated per antibody measured and calculated by operation E was 9.18. FIG. 14A showed a detection graph of aggregates, and a content of the aggregates in the antibody-drug conjugate Human IgG-LP2 measured by operation H was 2.92%. FIG. 14B showed a detection graph of the hydrophobic interaction chromatography of the antibody-drug conjugate Human IgG-LP2, and a retention time of the antibody-drug conjugate Human IgG-LP2 measured by operation I was 6.506 min.

### Comparative Example 8: Preparation of Antibody-Drug Conjugate Human IgG-LP3 (DAR 8)

Reduction of the antibody: a medium of the antibody was exchanged with PBS7.0/EDTA by operation B (an extinction coefficient of the antibody at 280 nm was 1.35 mLmg⁻¹ cm⁻¹) and operation C in Example 4, and a concentration of the antibody after medium exchange was 18 mg/mL. 0.672 mL of 5 mM TCEP solution (corresponding to 7 times equivalent of the content of the antibody) was added to 4 mL of human IgG protein aqueous solution (purchased from Beijing Solarbio Science & Technology Co., Ltd., product No. SP001), and 1.44 mL of 50 mM PBS7.0 and 1.088 mL of deionized water were added thereto at the same time. After confirming that the pH of the solution was 7.0±0.1, the mixture was placed in an environment of 37° C. to react for 2 hours. Resource of human IgG protein was the same as that of human IgG protein in Comparative Example 4.

Conjugation of antibody and linker-payload: the above mixed solution was incubated at 4° C. for 10 minutes. Linker-payload LP-3 was dissolved in DMA, and 0.672 mL (corresponding to 14 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D in Example 4 to obtain antibody-drug conjugate Human IgG-LP3.

Characterization of the antibody-drug conjugate: the obtained antibody-drug conjugate was characterized using operation E (ε_{D,280}=5186 and ε_{D,370}=13688), operation H, and operation I in Example 4.

The concentration of the antibody-drug conjugate measured and calculated by operation E was 7.42 mg/mL, and an average number of payloads conjugated per antibody measured and calculated by operation E was 7.28. FIG. 15A showed a detection graph of aggregates, and a content of the aggregates in antibody-drug conjugate Human IgG-LP3 measured by operation H was 5.37%. FIG. 15B showed a detection graph of the hydrophobic interaction chromatography of antibody-drug conjugate Human IgG-LP3, and a retention time of antibody-drug conjugate Human IgG-LP3 measured by operation I was 6.450 min.

Comparative Example 9: Preparation of Antibody-Drug Conjugate Hu1H2-2-GGFG-DXD (DAR 8)

Reduction of the antibody: reduction in Comparative Example 9 was the same as that of Example 6. The sequence of Hu1H2-2 antibody was the same as that of Hu1H2-2 antibody in Example 6.

Conjugation of antibody and linker-payload: the above mixed solution was incubated at 4° C. for 10 minutes. Linker-payload GGFG-DXd was dissolved in DMA, and 72 μL (corresponding to 18 times equivalent of the content of the antibody) of the solution then was added to the mixed solution. The reaction of the mixed solution continued at 22° C. for 30 minutes.

Purification of the antibody-drug conjugate: the above reaction solution was purified by the method of operation D to obtain antibody-drug conjugate Hu1H2-2-GGFG-DXd (referred to as “Hu1H2-2-DXD”).

Characterization of the antibody-drug conjugate: the obtained antibody-drug conjugate was characterized using the above operation E (ε_{D,280}=5178 and ε_{D,370}=20217), operation H, and operation I.

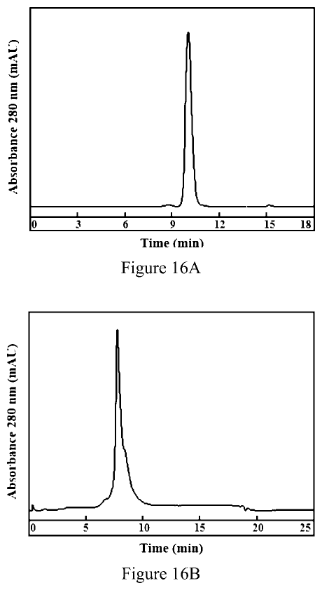

The concentration of the antibody-drug conjugate measured and calculated by operation E was 6.53 mg/mL, and an average number of payloads conjugated per antibody measured and calculated by operation E was 6.75. A content of the aggregates in the antibody-drug conjugate Hu1H2-2-DXD measured by operation H was 1.70%, as shown in FIG. 16A. A retention time of the antibody-drug conjugate Hu1H2-2-DXD measured by operation I was 7.764 min, as shown in FIG. 16B.

### Experimental Example 1: Endocytosis of 02-1-LP1 by Tumor Cells of Overexpressing MUC18

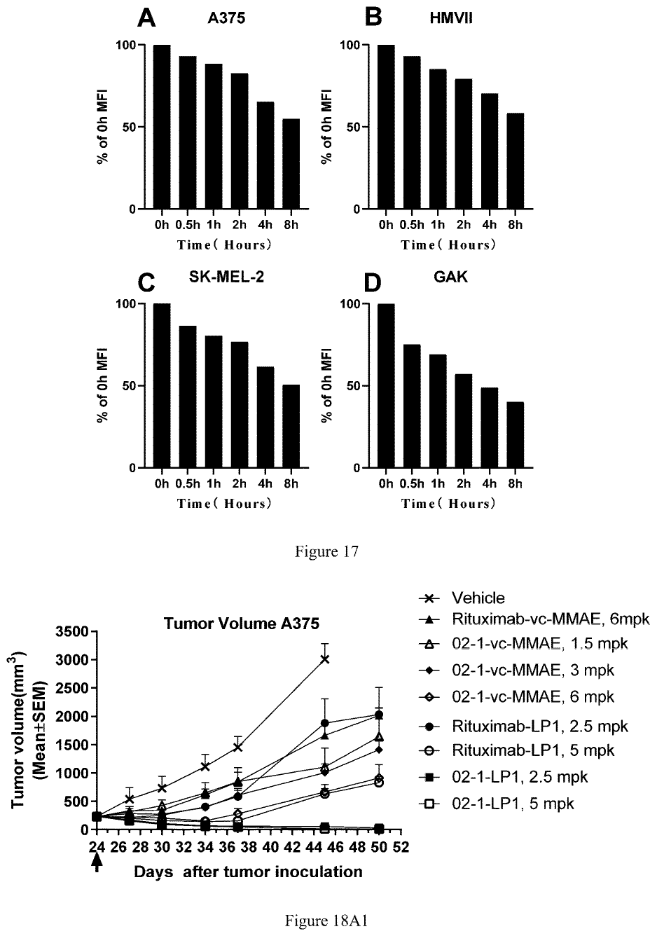

50 μg/mL 02-1-LP1 was combined with A375 cell (purchased from Shanghai Institute for Biological Sciences, Chinese Academy of Sciences), HMVII cell (purchased from Center for Type Culture Collection, Biovector NTCC), SK-MEL-2 cell (purchased from Shanghai Xunqing Biotechnology Co., Ltd.), and GAK cell (purchased from Center for Type Culture Collection, Biovector NTCC), respectively. After washing off excess ADC, cells were cultured in an incubator of 37° C. At 0, 0.5, 1, 2, 4, and 8 hours, a median fluorescence intensity (MFI) was measured by flow cytometry. As shown in FIG. 17, 02-1-LP1 antibody can be endocytosed by A375 (A) cell, HMVII (B) cell, SK-MEL-2 (C) cell, and GAK (D) cell.

### Experimental Example 2: In Vivo Inhibitory Effect of A375 Xenograft Tumor and Effect Thereof on Body Weight Change of Mice

6-week-old Balb/c mice were purchased from Jiangsu GemPharmatech Co. Ltd., and each mouse was subcutaneously inoculated with 4 million A375 cells to construct a MUC18-positive A375 melanoma mouse xenograft model. On the 24th day after inoculation (an average tumor volume was about 230 mm³), Rituximab-vc-MMAE (6 mpk (mg/kg)) prepared in Comparative Example 2, 02-1-vc-MMAE (1.5, 3, 6 mpk) prepared in Comparative Example 1, Rituximab-LP1 (2.5, 5 mpk) prepared in Comparative Example 3, and 02-1-LP1 (2.5, 5 mpk) prepared in Example 5, were intravenously injected, respectively; and a vehicle group was set as a negative control. After administration, a tumor volume was measured twice a week with a vernier caliper, and the tumor volume was calculated according to the following formula: TV=(length×width) 2/2. Since A375 cell hardly express CD20, Rituximab was used as an isotype control for 02-1 antibody at that time.

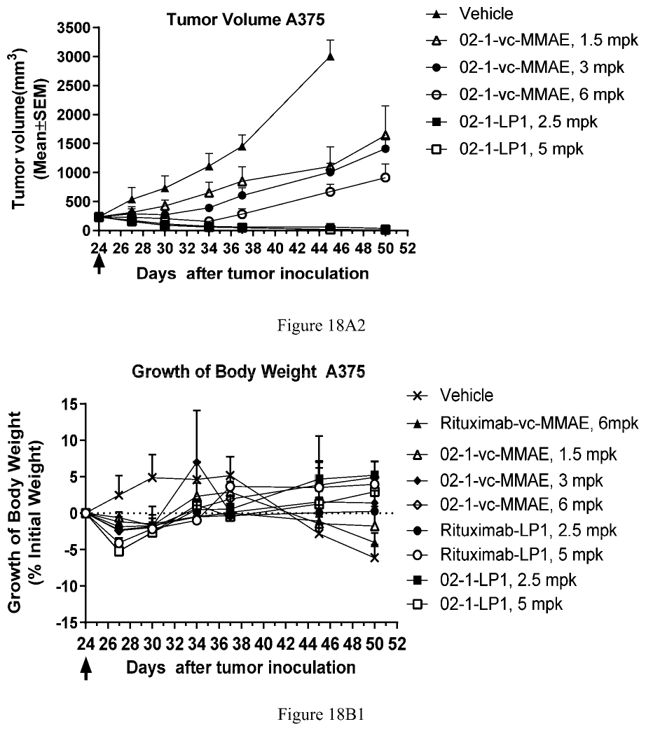

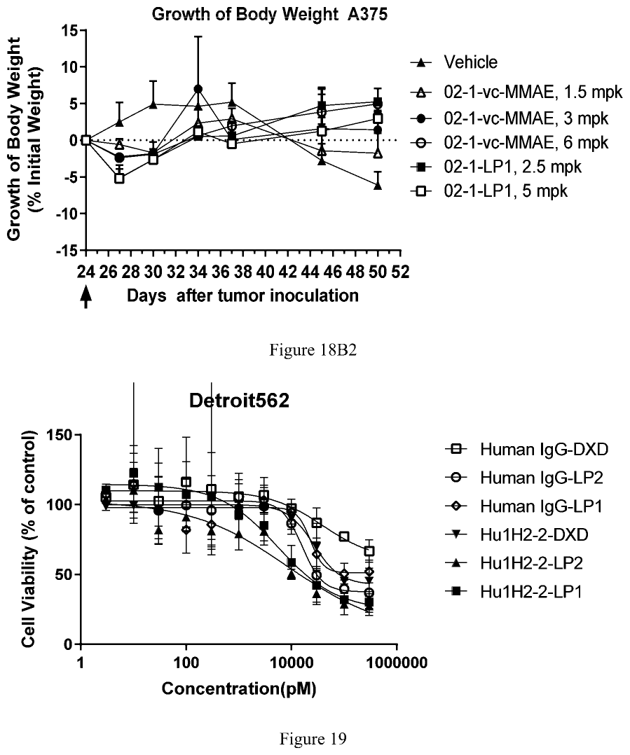

FIG. 18A1 and FIG. 18A2 showed growth inhibitory effects of MUC18 targeting antibody-drug conjugates 02-1-LP1 and 02-1-vc-MMAE on A375 xenograft. FIG. 18A1 showed that 02-1-vc-MMAE reduced a tumor growth rate, while 02-1-LP1 caused significant elimination of tumor volume. FIG. 18A2 was a partial view of FIG. 18A1, showing curves of 02-1-LP1, 02-1-vc-MMAE and Vehicle groups to further clarify trend graphs of the active ingredients.

FIG. 18B1 and FIG. 18B2 showed that MUC18 targeting antibody-drug conjugates 02-1-LP1 and 02-1-vc-MMAE did not significantly affect the body weight of the mice. FIG. 18B2 was a partial view of 18B1, showing the curves of the 02-1-LP1, 02-1-vc-MMAE and Vehicle groups to further clarify the trend graphs of the active ingredients.

### Experimental Example 3: Toxicological Experiments of 02-1-LP1 and 02-1-Vc-MMAE on Cynomolgus Monkeys

Experiment of repeated administration of 02-1-LP1 (prepared in Example 5) to cynomolgus monkeys: 6 cynomolgus monkeys (purchased from Guangxi Frontier Biotechnology Co., Ltd.) were randomly divided into 3 groups, and there were one male and one female in each group. Vehicle, 10 mg/kg of 02-1-LP1, and 30 mg/kg of 02-1-LP1 were infused intravenously on the 1st day, the 22nd day and the 43rd day, respectively. During the experiment, animals were observed for any abnormalities, and blood samples thereof were collected for hematology and blood biochemical index analysis. On the 50th day, the animals were euthanized and sampled for pathological analysis. As shown in Table 6, in the present experiment, all doses of the test substance did not cause death of the animals. Target organs related to the test substance were reticulocytes, digestive tract, kidney, and spleen. HNSTD (maximum dose without severe toxicity) was 30 mg/kg.

Experiment of repeated administration of 02-1-vc-MMAE (prepared in Comparative Example 1) to cynomolgus monkeys: 6 cynomolgus monkeys were randomly divided into 3 groups, and there were one male and one female in each group. 3 mg/kg 02-1-vc-MMAE, 6 mg/kg 02-1-vc-MMAE, and 10 mg/kg 02-1-vc-MMAE were infused intravenously on the 1st day, the 22nd day, respectively. During the experiment, animals were observed for any abnormalities, and blood samples thereof were collected for hematology and blood biochemical index analysis. On the 43rd day, the animals were euthanized and sampled for pathological analysis. As shown in Table 6, in the present experiment, two animals in the high-dose group (10 mg/kg) died on the 10th day after the first dose, and no death occurred in the 3 mg/kg and 6 mg/kg groups. Target organs related to the test substance were reticulocytes, leukocytes, and skin. HNSTD (maximum dose without severe toxicity) was 6 mg/kg.

TABLE 6
Summary of toxicological results of repeated administration
of 02-1-LP1 and 02-1-vc-MMAE to cynomolgus monkeys

| Experimental | | |
|---|---|---|
| animal | cynomolgus monkey | cynomolgus monkey |
| Test substance | 02-1-vc-MMAE | 02-1-LP1 |
| | 3, 6, and 10 mg/kg | 0, 10, and 30 mg/kg |
| | Intravenous injection | Intravenous injection |
| | Dosing on the 1st day and the 22nd | Dosing on the 1st day, the 22nd day, and |
| | day (2 times in total) | the 43rd day (3 times in total) |
| Number of | One male and one male in each | One male and one male in each group |
| animals | group | |
| Lethal dose | 10 mg/kg | >30 mg/kg |
| Body weight | ADC did not cause body weight | ADC did not cause body weight change |
| change | change in animals | in animals |
| Hematology | 3 mg/kg: Slight increase in | ≥10 mg/kg: Fluctuations in the number |
| index | reticulocytes | of reticulocytes |
| | 6 mg/kg: Slight decrease in leukocyte | |
| | and hemoglobin; fluctuations in the | |
| | number of reticulocytes | |
| Blood | ≤6 mg/kg: Normal | ≤30 mg/kg: Normal |
| biochemistry | | |
| Targeted toxic | 6 mg/kg: Sternal bone marrow | 30 mg/kg: Digestive tract, kidney, and |
| organs at | 10 mg/kg: | spleen |
| HNSTD dose | Digestive tract, inguinal | |
| | lymph node, and skin | |
| HNSTD* | 6 mg/kg | 30 mg/kg |

HNSTD*: Maximum dose without severe toxicity

The results showed that the safety of 02-1-LP1 prepared in Example 5 was significantly better than that of 02-1-vc-MMAE prepared in Comparative Example 1.

In conjunction with Experimental Example 2, 02-1-LP1 had a higher tolerable dose and better safety than those of 02-1-vc-MMAE, and at the same time, a lower dose of 02-1-LP1 can achieve better anti-tumor effect.

### Experimental Example 4: In Vitro Killing Assay of ADC on Detroit562 Cells

Detroit 562 cells (purchased from Nan Jing cobioer biosciences Co., Ltd.) were cultured so that a cell density reached to 80%. The cells were collected, plated in a 96-well plate, and the cell density was adjusted to 2-5×10⁴/ml. Each well was plated with 100 μL of cells, and the ADC molecules were serially diluted 3 times with an initial concentration of 300 nM. After the dilution was completed, the ADC molecules were added to the cell culture medium and cultured for 5 days, during which the apoptosis of the cells was observed regularly. After 5 days, 15 μL of the CCK-8 kit stock solution was added to the 96-well plate and reacted in an incubator of 37° C. for 0.5-2 h. An absorbance at 450 nm was measured, and cell survival curves were drawn based on OD (optical density) readings and ADC dilution gradients.

Human IgG-DXD prepared in Comparative Example 4, Human IgG-LP1 prepared in Comparative Example 5, Human IgG-LP2 prepared in Comparative Example 7, Hu1H2-2-LP1 prepared in Example 6, Hu1H2-2-LP2 prepared in Example 8, and Hu1H2-2-DXD prepared in Comparative Example 9 were selected as ADC molecules. Wherein Human IgG was used as the isotype control of Hu1H2-2 antibody, and DXD was used as positive drug.

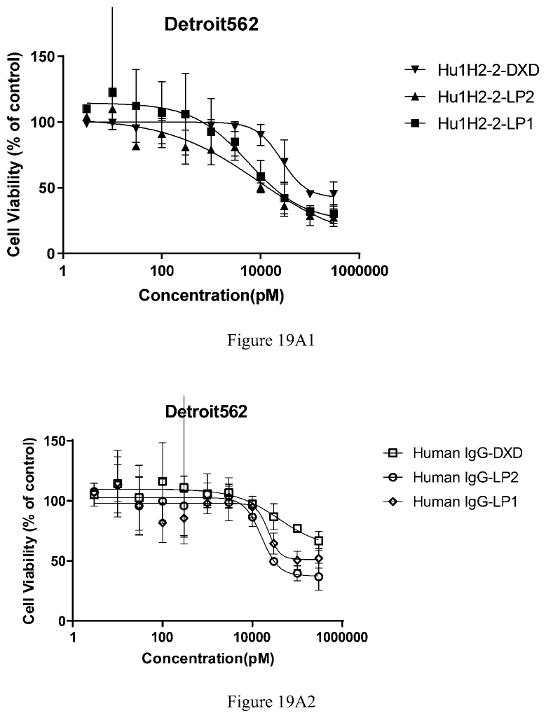

FIG. 19 showed the in vitro killing effect of the above ADC molecules on the head and neck squamous cell carcinoma cell line Detroit 562, and FIG. 19A1 and FIG. 19A2 were partial views of FIG. 19. FIG. 19A1 showed that, compared with the positive control Hu1H2-2-DXD, Hu1H2-2-LP2 and Hu1H2-2-LP1 of the present disclosure had more excellent killing effects in vitro on Detriot 562. FIG. 19A2 showed that, Human IgG-LP2 and Human IgG-LP1 had better killing effects in vitro than Human IgG-DXD, indicating that compared to GGFG-DXd, linker-payload LP-1 and linker-payload LP-2 had more excellent killing effects in vitro on Detriot562.

### Experimental Example 5: Effects of ADC on the In Vivo Efficacy of CDX Mouse Model of Detroit 562 and Body Weight Change of Mouse

6-week-old Balb/c nude mice were purchased from Jiangsu GemPharmatech Co., Ltd. Each mouse was subcutaneously inoculated with 5 million Detroit 562 cells to construct CDX (cell derived xenograft) mouse model. On the 10th day after inoculation (an average tumor volume was about 150 mm³), Human IgG-LP1 (10 mg/kg) prepared in Comparative Example 5, Hu1H2-2-LP1 (10 mg/kg) prepared in Example 6, and Hu1H2-2-LP2 (10 mg/kg) prepared in Example 8 were intravenously injected, respectively. After administration, a tumor volume was measured twice a week with a vernier caliper, and the tumor volume was calculated according to the following formula: TV=(length×width)²/2.

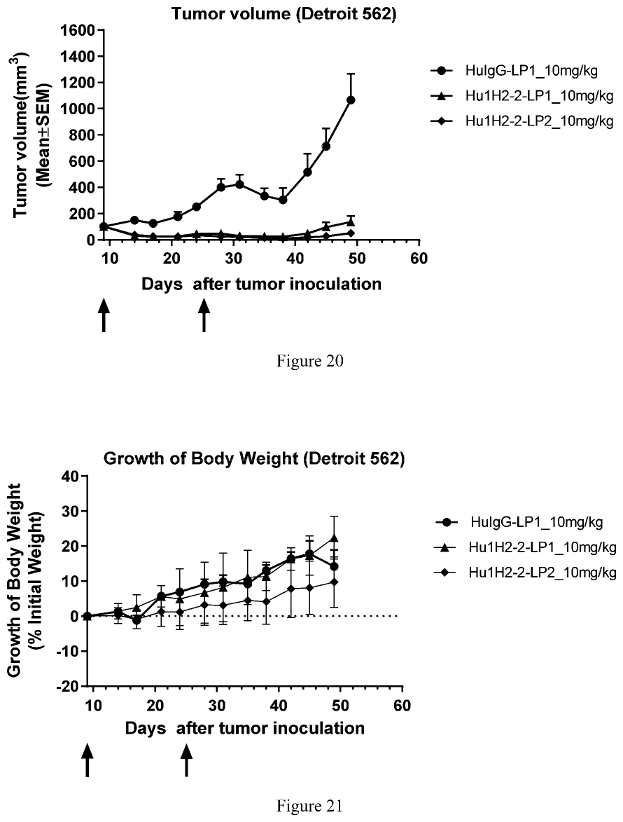

FIG. 20 showed the in vivo efficacy data of Hu1H2-2-LP1, Hu1H2-2-LP2, and Human IgG-LP1 on head and neck squamous cell carcinoma model mice constructed with Detroit562 cells. The results showed that Hu1H2-2-LP1 and Hu1H2-2-LP2 could effectively inhibit tumor growth in mice.

FIG. 21 showed effects of Hu1H2-2-LP1, Hu1H2-2-LP2, and Human IgG-LP1 on body weight of mice. During the experimental period, the body weight of mice in Hu1H2-2-LP1 group and Hu1H2-2-LP2 group continued to increase, while the body weight of Human IgG-LP1 in the control group fluctuated. The results showed that Hu1H2-2-LP1 and Hu1H2-2-LP2 had less effects on the body weight change of mice.

### Experimental Example 6: Effects of ADC on the In Vivo Efficacy of CDX Mouse Model of PC-9 and Body Weight Change of Mouse

6-week-old Balb/c nude mice were purchased from Jiangsu GemPharmatech Co. Ltd. Each mouse was subcutaneously inoculated with five million PC-9 cells (purchased from Meisen Chinese Tissue Culture Collections) to obtain CDX model. On the 13th day after inoculation (an average tumor volume was about 200 mm³), Human IgG-LP1 prepared in Comparative Example 5, Human IgG-LP1 (DAR4) prepared in Comparative Example 6, Human IgG-LP3 prepared in Comparative Example 8, Hu1H2-2-LP1 prepared in Example 6, Hu1H2-2-LP1 (DAR4) prepared in Example 7, and Hu1H2-2-LP3 prepared in Example 9 were intravenously injected, respectively. The administration doses were all 10 mpk (mg/kg), once a week for two times (QW×2). After administration, a tumor volume was measured twice a week with a vernier caliper, and the tumor volume was calculated according to the following formula: TV=(length×width)²/2.

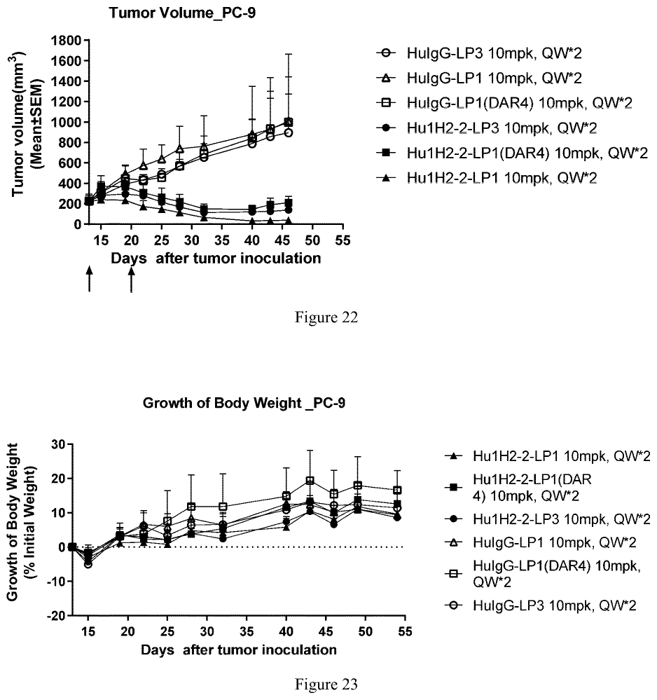

FIG. 22 showed the in vivo efficacy data of the above ADC on lung cancer model mice constructed with PC-9 cells. The results showed that Hu1H2-2-LP1, Hu1H2-2-LP1 (DAR4), and Hu1H2-2-LP3 can effectively inhibit tumor growth in mice.

FIG. 23 showed the effect of the above ADC on body weight of lung cancer model mice constructed with PC-9. The results showed that Hu1H2-2-LP1, Hu1H2-2-LP1 (DAR4), and Hu1H2-2-LP3 had little effects on the body weight of mice and had no obvious digestive tract toxicity.

### Experimental Example 7: In Vivo Inhibitory Effect on SCC-9 Human Head and Neck Squamous Cell Carcinoma CDX Model and Effect Thereof on Body Weight Change of Mice

6-week-old Balb/c nude mice were purchased from Shanghai BK/KY Biotechnology Co., Ltd. Each mouse was subcutaneously inoculated with eight million SCC-9 cells (purchased from ATCC) to obtain CDX model. On the 29th day after inoculation (an average tumor volume was about 200 mm³), Rituximab-LP1 (10 mpk) prepared in Comparative Example 3, and 02-1-LP1 (2.5, 5, 10 mpk) prepared in Example 5 were intravenously injected, respectively. DPBS (Dulbecco's Phosphate-Buffered Saline) was negative control. The administration was once a week for two times (QW×2). After administration, a tumor volume was measured twice a week with a vernier caliper, and the tumor volume was calculated according to the following formula: TV=(length×width)²/2.

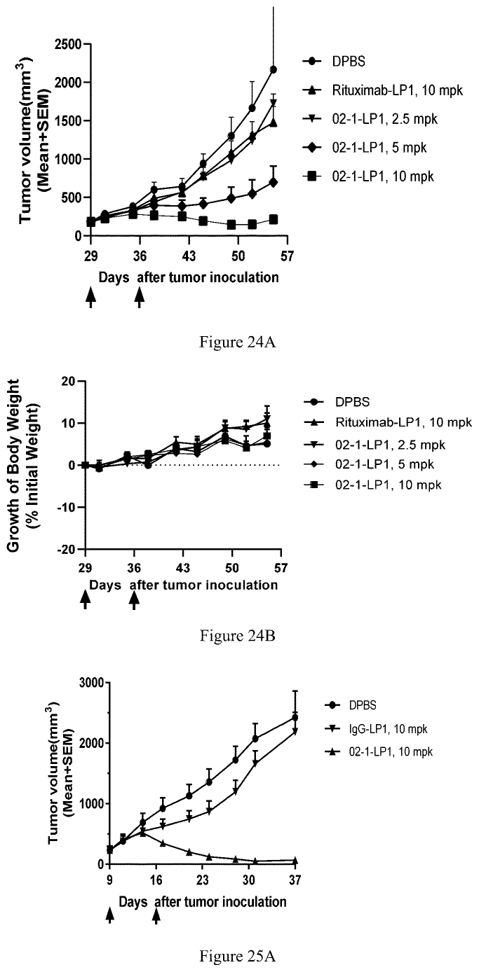

FIG. 24A showed that 02-1-LP1 can effectively inhibit tumor growth in SCC-9 CDX model. FIG. 24B showed that 02-1-LP1 had little effects on the body weight of mice in SCC-9 model.

### Experimental Example 8: In Vivo Inhibitory Effect on Huh-7 Human Liver Cancer CDX Model and Effect Thereof on Body Weight Change of Mice

6-week-old Balb/c nude mice were purchased from Shanghai BK/KY Biotechnology Co., Ltd. Each mouse was subcutaneously inoculated with five million Huh-7 cells (purchased from Shanghai BioGene Biotech Co., Ltd,) to obtain CDX model. On the 9th day after inoculation (an average tumor volume was about 230 mm³), Human IgG-LP1 prepared in Comparative Example 5, and 02-1-LP1 prepared in Example 5 were intravenously injected, respectively. The administration doses were all 10 mpk, once a week for two times (QW×2). After administration, a tumor volume was measured twice a week with a vernier caliper, and the tumor volume was calculated according to the following formula: TV=(length×width)²/2.

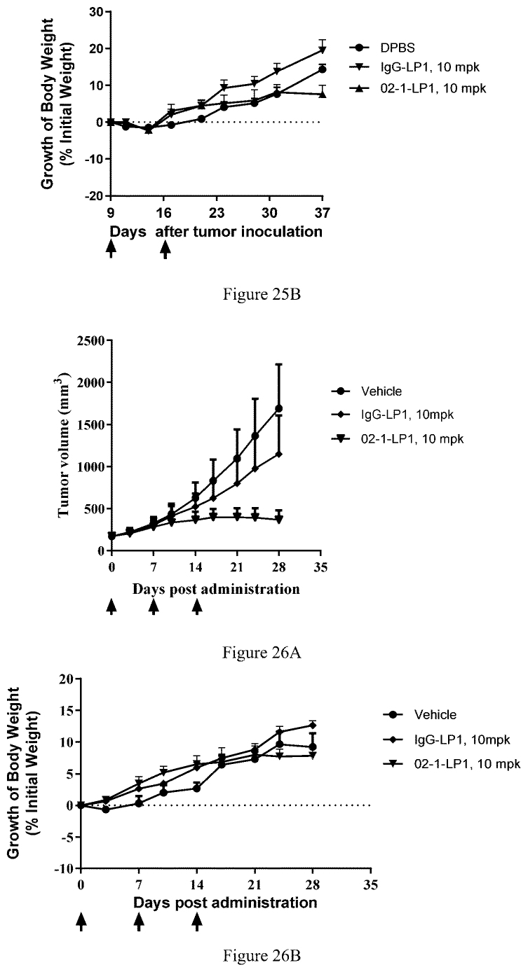

FIG. 25A showed that 02-1-LP1 can effectively inhibit tumor growth in Huh-7 CDX model. FIG. 25B showed that 02-1-LP1 had little effects on the body weight of mice in Huh-7 model.

### Experimental Example 9: In Vivo Inhibitory Effect on LD1-0015-200617 Human Esophagus Squamous Cell Carcinoma PDX Model and Effect Thereof on Body Weight Change of Mice

LD1-0015-200617 (LideBiotech CO., LTD) is a human esophagus squamous cell carcinoma patient derived xenograft model which expresses human MUC18 (H-Score=75). 6-8 weeks Nu/Nu mice were purchased from Beijing Vital River Laboratory Animal Technology Co., Ltd. Tumors of LD1-0015-200617 were sliced into about 3 mm×3 mm×3 mm fragments and implanted subcutaneously into the flank of mice. On day 39 (mean tumor size ˜171 mm³) following implanting, Human IgG-LP1 prepared in Comparative Example 5, and 02-1-LP1 prepared in Example 5 were intravenously injected, respectively. The administration doses were all 10 mpk, once a week for three times (QW×3). After administration, a tumor volume was measured twice a week with a vernier caliper, and the tumor volume was calculated according to the following formula: TV=(length×width)²/2.

FIG. 26A showed that 02-1-LP1 can effectively inhibit tumor growth in LD1-0015-200617 PDX model. FIG. 26B showed that 02-1-LP1 had little effects on the body weight of mice in LD1-0015-200617 PDX model.

### Experimental Example 10: In Vivo Inhibitory Effect on LD1-0016-390730 Human Esophagus Adenocarcinoma PDX Model and Effect Thereof on Body Weight Change of Mice

LD1-0016-390730 (LideBiotech CO., LTD) is a human esophagus adenocarcinoma patient derived xenograft model which expresses human MUC18 (H-Score=115). 6-8 weeks Nu/Nu mice were purchased from Beijing Vital River Laboratory Animal Technology Co., Ltd. Tumors of LD1-0016-390730 were sliced into about 3 mm×3 mm×3 mm fragments and implanted subcutaneously into the flank of mice. On day 45 (mean tumor size ˜191 mm³) following implanting, 02-1-LP1 prepared in Example 5 was intravenously injected. The administration dose was 10 mpk, once a week for two times (QW×2). After administration, a tumor volume was measured twice a week with a vernier caliper, and the tumor volume was calculated according to the following formula: TV=(length×width)²/2.

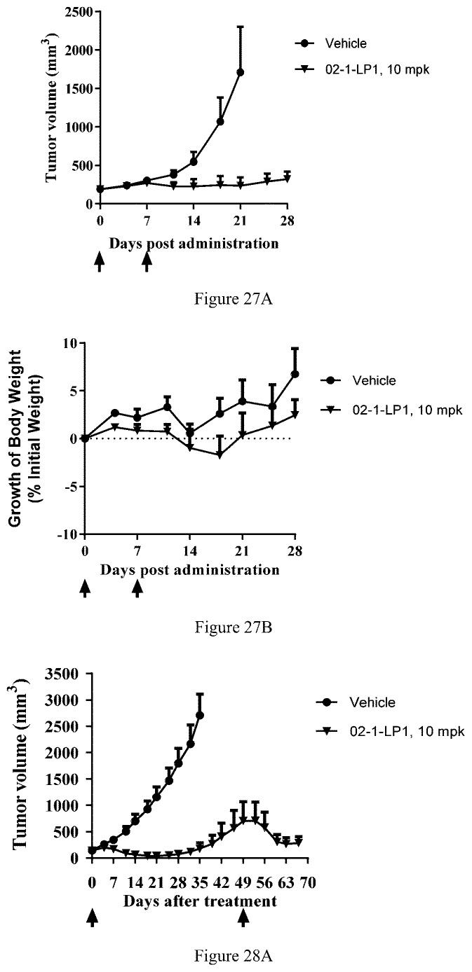

FIG. 27A showed that 02-1-LP1 can effectively inhibit tumor growth in LD1-0016-390730 PDX model. FIG. 27B showed that 02-1-LP1 had little effects on the body weight of mice in LD1-0016-390730 PDX model.

### Experimental Example 11: In Vivo Inhibitory Effect on LD1-2025-362797 Human Small Cell Lung Cancer PDX Model and Effect Thereof on Body Weight Change of Mice

LD1-2025-362797 (LideBiotech CO., LTD) is a human small cell lung cancer patient derived xenograft model which expresses human MUC18 (H-Score=110). 6-8 weeks Nu/Nu mice were purchased from Beijing Vital River Laboratory Animal Technology Co., Ltd. Tumors of LD1-2025-362797 were sliced into about 3 mm×3 mm×3 mm fragments and implanted subcutaneously into the flank of mice. On day 21 (mean tumor size ˜144 mm³) following implanting, a first dose of 10 mpk 02-1-LP1 prepared in Example 5 was intravenously injected. And a second dose of 10 mpk 02-1-LP1 was also intravenously injected when the tumor volume regrew to about 700 mm³ (49th day after the first dose). After administration, a tumor volume was measured twice a week with a vernier caliper, and the tumor volume was calculated according to the following formula: TV=(length×width)²/2.

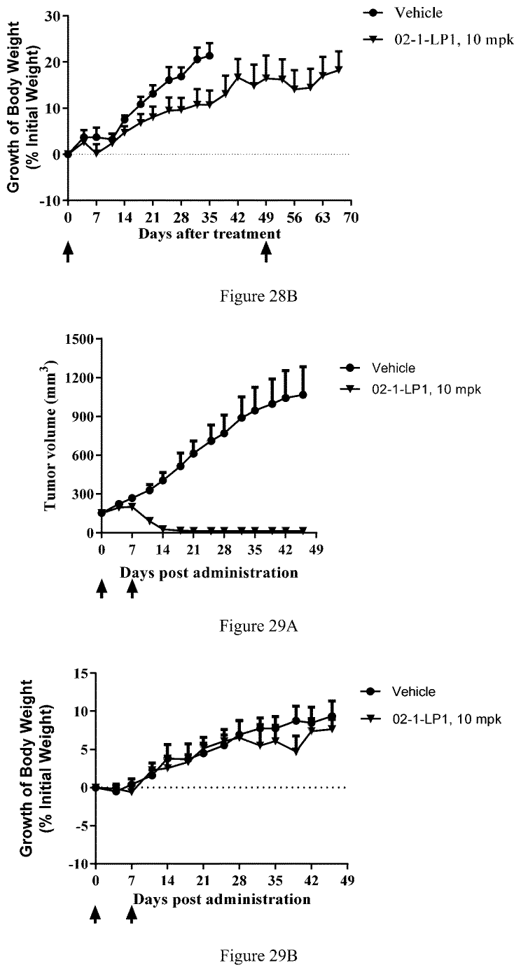

FIG. 28A showed that 02-1-LP1 can effectively inhibit tumor growth in LD1-2025-362797 PDX model. Notably, the tumor decreased again after the second dose of 02-1-LP1, indicating that 02-1-LP1 is still active against the regrown tumor. FIG. 28B showed that 02-1-LP1 had little effects on the body weight of mice in LD1-2025-362797 PDX model.

### Experimental Example 12: In Vivo Inhibitory Effect on LD1-2009-362263 Human Triple Negative Breast Cancer PDX Model and Effect Thereof on Body Weight Change of Mice

LD1-2009-362263 (LideBiotech CO., LTD) is a human triple negative breast cancer patient derived xenograft model which expresses human MUC18 (H-Score=240). 6-8 weeks Nu/Nu mice were purchased from Beijing Vital River Laboratory Animal Technology Co., Ltd. Tumors of LD1-2009-362263 were sliced into about 3 mm×3 mm×3 mm fragments and implanted subcutaneously into the flank of mice. On day 49 (mean tumor size ˜152 mm³) following implanting, 02-1-LP1 prepared in Example 5 was intravenously injected. The administration dose was 10 mpk, once a week for two times (QW×2). After administration, a tumor volume was measured twice a week with a vernier caliper, and the tumor volume was calculated according to the following formula: TV=(length×width)²/2.

FIG. 29A showed that 02-1-LP1 can effectively inhibit tumor growth in LD1-2009-362263 PDX model. FIG. 29B showed that 02-1-LP1 had little effects on the body weight of mice in LD1-2009-362263 PDX model.

### Experimental Example 13: In Vivo Inhibitory Effect on LD1-0060-200770 Human Cholangiocarcinoma PDX Model and Effect Thereof on Body Weight Change of Mice

LD1-0060-200770 (LideBiotech CO., LTD) is a human cholangiocarcinoma patient derived xenograft model which expresses human MUC18 (H-Score=15). 6-8 weeks Nu/Nu mice were purchased from Beijing Vital River Laboratory Animal Technology Co., Ltd. Tumors of LD1-0060-200770 were sliced into about 3 mm×3 mm×3 mm fragments and implanted subcutaneously into the flank of mice. On day 42 (mean tumor size ˜155 mm³) following implanting, 02-1-LP1 prepared in Example 5 was intravenously injected. The administration dose was 10 mpk, once a week for two times (QW×2). After administration, a tumor volume was measured twice a week with a vernier caliper, and the tumor volume was calculated according to the following formula: TV=(length×width)²/2.

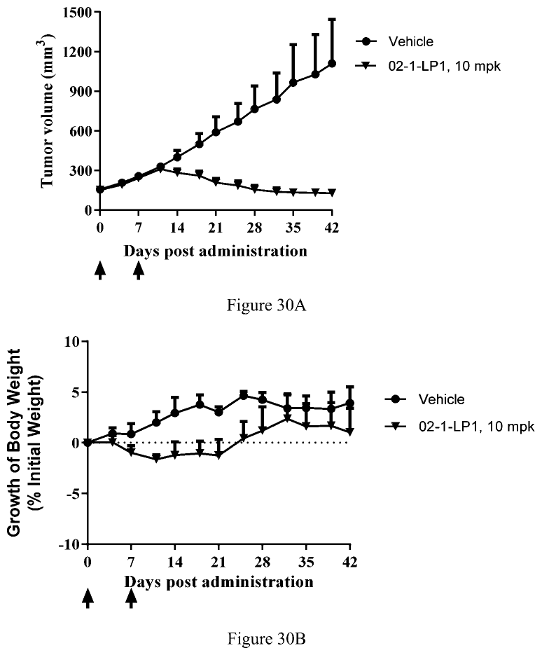

FIG. 30A showed that 02-1-LP1 can effectively inhibit tumor growth in LD1-0060-200770 PDX model. FIG. 30B showed that 02-1-LP1 had little effects on the body weight of mice in LD1-0060-200770 PDX model.

### Experimental Example 14: In Vivo Inhibitory Effect on OV-10-0073 Human Ovarian Cancer PDX Model and Effect Thereof on Body Weight Change of Mice

OV-10-0073 (WuXi AppTech (Shanghai) Co., Ltd) is a human ovarian cancer patient derived xenograft model which expresses human MUC18 (H-Score=40). 6-8 weeks BALB/c nude mice were purchased from Beijing Vital River Laboratory Animal Technology Co., Ltd. Tumors of OV-10-0073 were sliced into about 30 mm³ fragments and implanted subcutaneously into the flank of mice. On day 34 (mean tumor size ˜185 mm³) following implanting, a single dose of Human IgG-LP1 (2.5, 5 mpk) prepared in Comparative Example 5, and 02-1-LP1 (1.25, 2.5, 5 mpk) prepared in Example 5 were intravenously injected, respectively. After administration, a tumor volume was measured twice a week with a vernier caliper, and the tumor volume was calculated according to the following formula: TV=(length×width)²/2.

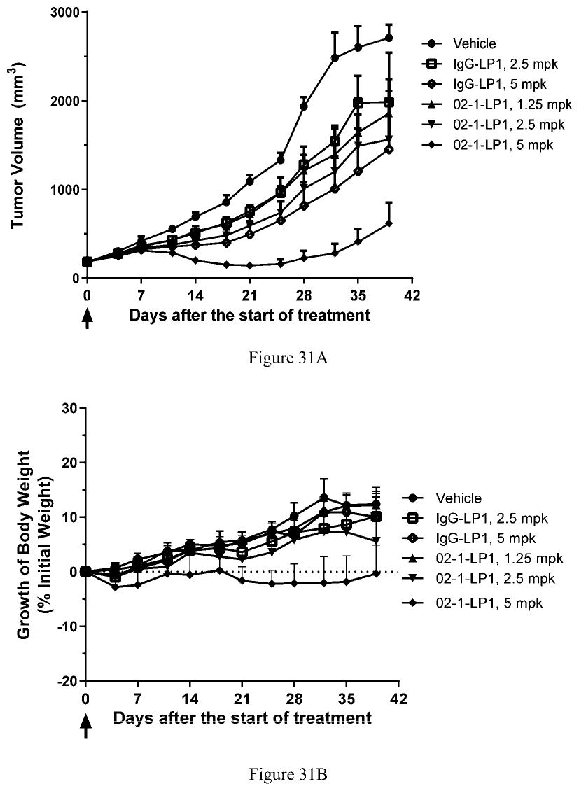

FIG. 31A showed that 02-1-LP1 can effectively inhibit tumor growth in OV-10-0073 PDX model. FIG. 31B showed that 02-1-LP1 had little effects on the body weight of mice in OV-10-0073 PDX model.

It can be seen from the above experimental results that, in the ADCs provided by the present disclosure, after bonding with exatecan, the linker is coupled with the antibody targeting MUC18 or CD44v7/8 by a simple chemical method. Compared with conventional random conjugation methods, the DAR values (DAR8) of anti-MUC18 antibody-drug conjugate or anti-CD44v7/8 antibody-drug conjugate obtained by using such linker is higher. The HIC detection showed that the chromatographic peaks of the ADC provided by the present disclosure were narrower than those of the linker-payload of vc-MMAE or GGFG-DXd, suggesting that the prepared products were highly homogeneous and hydrophilic. Compared with conventional vc-MMAE and GGFG-DXd conjugates, the in vitro tumor cell proliferation inhibitory activity of the conjugates in the present disclosure is improved or maintained in terms of biological activity and safety.

The ADCs prepared in the present disclosure have specific MUC18 or CD44v7/8-dependent antitumor activity and have extremely high killing activity on tumor cells with high expression of MUC18 or CD44v7/8. The ADCs with MUC18 specificity prepared in the disclosure have an increased HNSTD, indicating improved safety and decreased toxic and side effects. Surprisingly, the results of experimental example 9-14 also showed that the ADCs with MUC18 specificity prepared in the disclosure show excellent killing activity on tumor cells with different expression levels (low, moderate, and high) of MUC18.

The ADCs prepared in the present disclosure have more excellent anti-tumor efficacy in vivo than that of the ADC conjugated with vc-MMAE or GGFG-DXd, and at the same time show significantly better safety than the ADC conjugated to vc-MMAE and have similar MTD (maximum tolerated dose) (Yusuke OgitaniClin, et al., Cancer Res. 2016 Oct. 15; 22 (20): 5097-5108) with GGFG-DXd ADC. It is expected that this drug conjugate will have a higher therapeutic window than the existing clinical molecules vc-MMAE or GGFG-DXd.

The ADCs provided by the present disclosure have better stability in vitro and are with reduced aggregation in the preparation process. The ADCs with linker of formula I have better stability in plasma, it is expected that the ADCs may have better drug metabolism properties in vivo, such as a longer half-life, a lower amount of free small molecule toxins, and the like.

1. An antibody-drug conjugate, or an isomer, an isotopic variant, a pharmaceutically acceptable salt, prodrug, solvate thereof, or combinations thereof, comprising
an anti-MUC18 or anti-CD44v7/8 antibody or antigen-binding fragment thereof, a payload, and a linker of formula I, wherein: 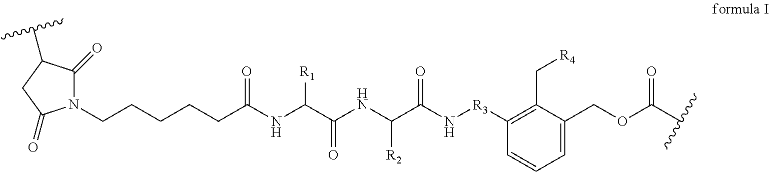
a succinimidyl group of the linker of formula I forms a thioether bond with a thiol group obtained by reduction of an interchain disulfide chain of the antibody or antigen-binding fragment thereof;
a carbonyl group in an ester group of the linker of formula I is connected to an amino group of the payload;
R₁ and R₂ are independently selected from hydrogen, methyl, and isopropyl group;
R₃ represents —(CR₅HCONH)n¹-(CH₂CONH)n²- or a single bond, R₅ is hydrogen or benzyl, n¹ represents an integer of 0 to 2, and n² represents an integer of 0 to 2;
R₄ represents a methylamino group or —(NCH₃COCH₂)n³-NCH₃COCH₃, and n³ represents an integer of 1 to 20.

2. The antibody-drug conjugate of claim 1, wherein R₄ represents —(NCH₃COCH₂)n³-NCH₃COCH₃, and n³ represents an integer of 8 to 15;
optionally, wherein R₄ represents —(NCH₃COCH₂)n³-NCH₃COCH₃, and n³ represents an integer of 10 to 12.

3. (canceled)

4. The antibody-drug conjugate of claim 1, wherein R₃ represents —(CR₅H₂CONH)n¹-(CH₂CONH)n²- or a single bond, R₅ is benzyl, n¹ represents 1 or 2, and n² represents 1 or 2;
R₄ represents —(NCH₃COCH₂)n³-NCH₃COCH₃, and n³ represents an integer of 8 to 15;
optionally, R₃ represents a single bond, R₄ represents —(NCH₃COCH₂)n³-NCH₃COCH₃, and n³ represents an integer of 8 to 15;
optionally, R₃ represents a single bond, and R₄ represents a methylamino group.

5. (canceled)

6. (canceled)

7. The antibody-drug conjugate of claim 1, wherein the linker is selected from any one of the following: 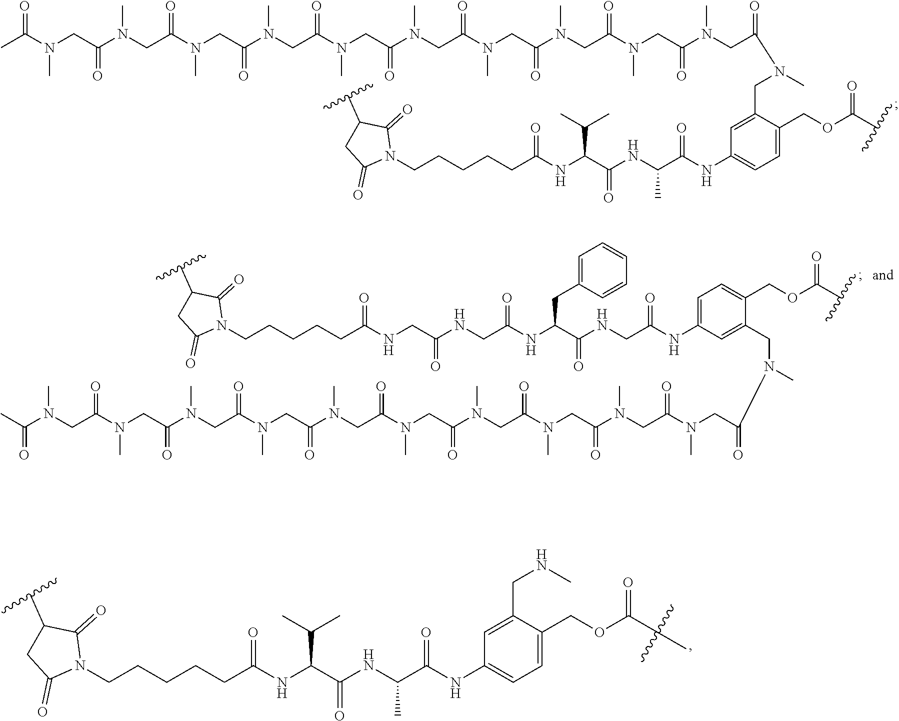 [SMILES: *C(=O)OCc1ccc(NC(=O)[C@H](C)NC(=O)[C@@H](NC(=O)CCCCCN2C(=O)CC(*)C2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *C(=O)OCc1ccc(NC(=O)CNC(=O)[C@H](Cc2ccccc2)NC(=O)CNC(=O)CNC(=O)CCCCCN2C(=O)CC(*)C2=O)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *C(=O)OCc1ccc(NC(=O)[C@H](C)NC(=O)[C@@H](NC(=O)CCCCCN2C(=O)CC(*)C2=O)C(C)C)cc1CNC]
or combinations thereof.

8. The antibody-drug conjugate of claim 1, wherein the payload is at least one selected from the group consisting of cytotoxic agents, labels, nucleic acids, radionuclides, hormones, immunomodulators, prodrug converting enzymes, ribonucleases, agonistic antibodies, antagonistic antibodies and fragments thereof, fusion proteins or derivatives thereof;
optionally, the cytotoxic agent includes a tubulin inhibitor and/or a topoisomerase inhibitor, the tubulin inhibitor includes auristatin or derivatives thereof, maytansine or derivatives thereof; and the topoisomerase inhibitor includes camptothecin and derivatives thereof.

9. (canceled)

10. The antibody-drug conjugate of claim 1, wherein the payload is exatecan of formula II, which is connected to the linker by a nitrogen atom of an amino group on a cyclohexane ring thereof,

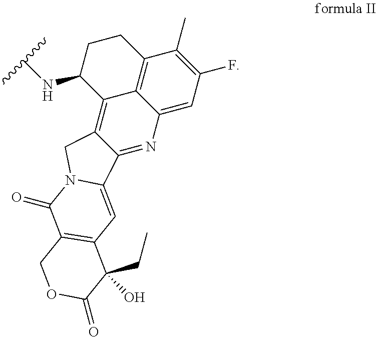 [SMILES: *N[C@H]1CCc2c(C)c(F)cc3nc4c(c1c23)Cn1c-4cc2c(c1=O)COC(=O)[C@]2(O)CC]

11. The antibody-drug conjugate of claim 1, wherein the anti-MUC18 antibody or antigen-binding fragment thereof comprises:
HCDR1 with amino acid sequence as shown in SEQ ID NO: 4, HCDR2 with amino acid sequence as shown in SEQ ID NO: 16, HCDR3 with amino acid sequence as shown in SEQ ID NO: 28, and LCDR1 with amino acid sequence as shown in SEQ ID NO: 38, LCDR2 with amino acid sequence STS, LCDR3 with amino acid sequence as shown in SEQ ID NO: 52;
HCDR1 with amino acid sequence as shown in SEQ ID NO: 1, HCDR2 with amino acid sequence as shown in SEQ ID NO: 11, HCDR3 with amino acid sequence as shown in SEQ ID NO: 23, and LCDR1 with amino acid sequence as shown in SEQ ID NO: 34, LCDR2 with amino acid sequence LAS, LCDR3 with amino acid sequence as shown in SEQ ID NO: 46;
HCDR1 with amino acid sequence as shown in SEQ ID NO: 1, HCDR2 with amino acid sequence as shown in SEQ ID NO: 12, HCDR3 with amino acid sequence as shown in SEQ ID NO: 23, and LCDR1 with amino acid sequence as shown in SEQ ID NO: 35, LCDR2 with amino acid sequence LAS, LCDR3 with amino acid sequence as shown in SEQ ID NO: 47;
HCDR1 with amino acid sequence as shown in SEQ ID NO: 2, HCDR2 with amino acid sequence as shown in SEQ ID NO: 13, HCDR3 with amino acid sequence as shown in SEQ ID NO: 24, and LCDR1 with amino acid sequence as shown in SEQ ID NO: 36, LCDR2 with amino acid sequence NAK, LCDR3 with amino acid sequence as shown in SEQ ID NO: 48;
HCDR1 with amino acid sequence as shown in SEQ ID NO: 3, HCDR2 with amino acid sequence as shown in SEQ ID NO: 14, HCDR3 with amino acid sequence as shown in SEQ ID NO: 25, and LCDR1 with amino acid sequence as shown in SEQ ID NO: 37, LCDR2 with amino acid sequence FAS, LCDR3 with amino acid sequence as shown in SEQ ID NO: 49;
HCDR1 with amino acid sequence as shown in SEQ ID NO: 4, HCDR2 with amino acid sequence as shown in SEQ ID NO: 15, HCDR3 with amino acid sequence as shown in SEQ ID NO: 26, and LCDR1 with amino acid sequence as shown in SEQ ID NO: 38, LCDR2 with amino acid sequence STS, LCDR3 with amino acid sequence as shown in SEQ ID NO: 50;
HCDR1 with amino acid sequence as shown in SEQ ID NO: 4, HCDR2 with amino acid sequence as shown in SEQ ID NO: 15, HCDR3 with amino acid sequence as shown in SEQ ID NO: 27, and LCDR1 with amino acid sequence as shown in SEQ ID NO: 39, LCDR2 with amino acid sequence STS, LCDR3 with amino acid sequence as shown in SEQ ID NO: 51;
HCDR1 with amino acid sequence as shown in SEQ ID NO: 5, HCDR2 with amino acid sequence as shown in SEQ ID NO: 17, HCDR3 with amino acid sequence as shown in SEQ ID NO: 29, and LCDR1 with amino acid sequence as shown in SEQ ID NO: 40, LCDR2 with amino acid sequence WAS, LCDR3 with amino acid sequence as shown in SEQ ID NO: 53;
HCDR1 with amino acid sequence as shown in SEQ ID NO: 6, HCDR2 with amino acid sequence as shown in SEQ ID NO: 18, HCDR3 with amino acid sequence as shown in SEQ ID NO: 29, and LCDR1 with amino acid sequence as shown in SEQ ID NO: 41, LCDR2 with amino acid sequence WAS, LCDR3 with amino acid sequence as shown in SEQ ID NO: 53;
HCDR1 with amino acid sequence as shown in SEQ ID NO: 7, HCDR2 with amino acid sequence as shown in SEQ ID NO: 19, HCDR3 with amino acid sequence as shown in SEQ ID NO: 30, and LCDR1 with amino acid sequence as shown in SEQ ID NO: 42, LCDR2 with amino acid sequence RTS, LCDR3 with amino acid sequence as shown in SEQ ID NO: 54;
HCDR1 with amino acid sequence as shown in SEQ ID NO: 8, HCDR2 with amino acid sequence as shown in SEQ ID NO: 20, HCDR3 with amino acid sequence as shown in SEQ ID NO: 31, and LCDR1 with amino acid sequence as shown in SEQ ID NO: 43, LCDR2 with amino acid sequence WAS, LCDR3 with amino acid sequence as shown in SEQ ID NO: 55;
HCDR1 with amino acid sequence as shown in SEQ ID NO: 9, HCDR2 with amino acid sequence as shown in SEQ ID NO: 21, HCDR3 with amino acid sequence as shown in SEQ ID NO: 32, and LCDR1 with amino acid sequence as shown in SEQ ID NO: 44, LCDR2 with amino acid sequence WAS, LCDR3 with amino acid sequence as shown in SEQ ID NO: 56; or
HCDR1 with amino acid sequence as shown in SEQ ID NO: 10, HCDR2 with amino acid sequence as shown in SEQ ID NO: 22, HCDR3 with amino acid sequence as shown in SEQ ID NO: 33, and LCDR1 with amino acid sequence as shown in SEQ ID NO: 45, LCDR2 with amino acid sequence LMS, LCDR3 with amino acid sequence as shown in SEQ ID NO: 57.

12. The antibody-drug conjugate of claim 1, wherein the anti-MUC18 antibody or antigen-binding fragment thereof comprises:
heavy chain variable region with amino acid sequence as shown in SEQ ID NO: 84, and light chain variable region with amino acid sequence as shown in SEQ ID NO: 85;
heavy chain variable region with amino acid sequence as shown in SEQ ID NO: 86, and light chain variable region with amino acid sequence as shown in SEQ ID NO: 87; or
heavy chain variable region with amino acid sequence as shown in SEQ ID NO: 88, and light chain variable region with amino acid sequence as shown in SEQ ID NO: 89, or conservative variants thereof.

13. The antibody-drug conjugate of claim 1, wherein the anti-CD44 v7/8 antibody or antigen-binding fragment thereof specifically binds to a binding peptide in human CD44 v7/8, wherein the binding peptide comprises an amino acid sequence as shown in SEQ ID NO: 90;
optionally, the anti-CD44 v7/8 antibody or antigen-binding fragment thereof does not bind to a binding peptide with an amino acid sequence as shown in SEQ ID NO: 91 and/or SEQ ID NO: 92 in human CD44 v7/8.

14. (canceled)

15. The antibody-drug conjugate of claim 13, wherein the anti-CD44 v7/8 antibody or antigen-binding fragment thereof comprises HCDR1 with amino acid sequence as shown in SEQ ID NO: 93, HCDR2 with amino acid sequence as shown in SEQ ID NO: 94, and HCDR3 with amino acid sequence as shown in SEQ ID NO: 95; and LCDR1 with amino acid sequence as shown in SEQ ID NO: 96, LCDR2 with amino acid sequence RAN, and LCDR3 with amino acid sequence as shown in SEQ ID NO: 97.

16. The antibody-drug conjugate of claim 13, wherein the anti-CD44v7/8 antibody or antigen-binding fragment thereof comprises:
heavy chain variable region with amino acid sequence as shown in SEQ ID NO: 100, and light chain variable region with amino acid sequence as shown in SEQ ID NO: 101; or
heavy chain variable region with amino acid sequence as shown in SEQ ID NO: 100, and light chain variable region with amino acid sequence as shown in SEQ ID NO: 104, or conservative variants thereof.

17. The antibody-drug conjugate of claim 1, wherein a DAR is 1 to 10;
optionally, the DAR is 4 to 10.

18. (canceled)

19. A preparation method of the antibody-drug conjugate of claim 1, or an isomer, an isotopic variant, a pharmaceutically acceptable salt, prodrug, solvate thereof, or combinations thereof, and a pharmaceutically acceptable excipient, comprising the following steps: reducing an anti-MUC18 or anti-CD44v7/8 antibody or antigen-binding fragment thereof such that disulfide bonds thereof are at least partially reduced, and reacting with a carbon atom at position 3 of maleimide-N-yl of the linker of formula III, 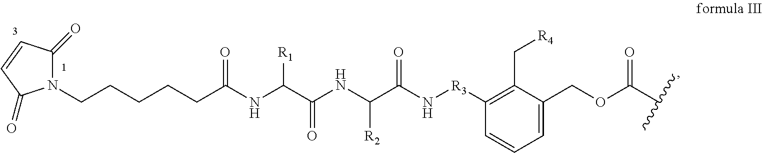
wherein
in the linker-payload, a carbonyl group in an ester group of the linker of formula III is connected to an amino group of the payload;
R₁ and R₂ are independently selected from hydrogen, methyl and isopropyl group;
R₃ represents —(CR₅HCONH)n¹-(CH₂CONH)n²- or a single bond, R₅ is hydrogen or benzyl, n¹ represents an integer of 0 to 2, n² represents an integer of 0 to 2; and
R₄ represents a methylamino group or —(NCH₃COCH₂)n³-NCH₃COCH₃, n³ represents an integer of 1 to 20.

20. The preparation method of claim 19, comprising: reacting the antibody or antigen-binding fragment thereof with a reducing agent in a buffer solution containing a chelating agent, and then adding a solution of the linker-payload, the linker having a structure of formula III, and adjusting pH of a reaction solution.

21. The preparation method of claim 19, wherein the payload is exatecan of formula II, which is connected to a carbonyl group of an ester group in formula III by a nitrogen atom of an amino group on a cyclohexane ring thereof, 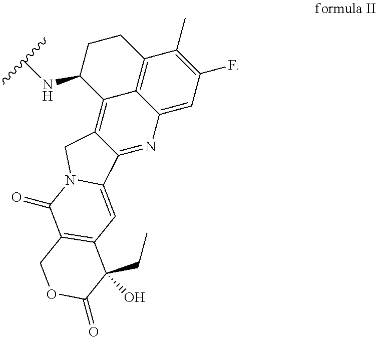 [SMILES: *N[C@H]1CCc2c(C)c(F)cc3nc4c(c1c23)Cn1c-4cc2c(c1=O)COC(=O)[C@]2(O)CC]
optionally, the antibody-drug conjugate has a DAR of 1 to 10.

22. (canceled)

23. The preparation method of claim 19, wherein
the anti-MUC18 antibody or antigen-binding fragment thereof comprises heavy chain variable region with amino acid sequence as shown in SEQ ID NO: 84, and light chain variable region with amino acid sequence as shown in SEQ ID NO: 85;
heavy chain variable region with amino acid sequence as shown in SEQ ID NO: 86, and light chain variable region with amino acid sequence as shown in SEQ ID NO: 87; or
heavy chain variable region with amino acid sequence as shown in SEQ ID NO: 88, and light chain variable region with amino acid sequence as shown in SEQ ID NO: 89, or conservative variants thereof;
the anti-CD44v7/8 antibody or antigen-binding fragment thereof comprises heavy chain variable region with amino acid sequence as shown in SEQ ID NO: 100, and light chain variable region with amino acid sequence as shown in SEQ ID NO: 101; or
heavy chain variable region with amino acid sequence as shown in SEQ ID NO: 100, and light chain variable region with amino acid sequence as shown in SEQ ID NO: 104, or conservative variants thereof.

24. A pharmaceutical composition comprising the antibody-drug conjugate of claim 1, or an isomer, an isotopic variant, a pharmaceutically acceptable salt, prodrug, solvate thereof, or combinations thereof, and a pharmaceutically acceptable excipient.

25. A kit, comprising the antibody-drug conjugate of claim 1, or an isomer, an isotopic variant, a pharmaceutically acceptable salt, prodrug, solvate thereof, or combinations thereof.

26. A method of manufacture, comprising using the antibody-drug conjugate of claim 1, in the manufacture of a therapeutic agent for diagnosis, prevention and treatment of neoplastic disease;
optionally, the neoplastic disease includes benign tumors and malignant tumors expressing MUC18 and/or CD44v7/8;
optionally, therapeutic agent targets to MUC18 and/or CD44v7/8.

27. (canceled)

28. (canceled)

29. (canceled)

30. A method of diagnosing, preventing, and treating neoplastic diseases, comprising administering to a subject a therapeutically effective amount of a therapeutic agent, wherein the therapeutic agent includes the antibody-drug conjugate of claim 1,
optionally, the neoplastic disease includes melanoma, pharyngeal cancer, triple-negative breast cancer, esophageal adenocarcinoma, esophagus squamous cell carcinoma, liver cancer, ovarian cancer, cervical cancer, urothelial cancer, bladder neuroendocrine tumor, small cell lung cancer, non-small cell lung cancer, cutaneous squamous cell carcinoma, cholangiocarcinoma, metastatic pancreatic carcinoma, lung squamous cell carcinoma, head and neck squamous cell carcinoma and/or esophageal squamous cell carcinoma.


SEQUENCE LISTING (104 sequences)

SEQ ID NO: 1 - protein, 9 aa, synthetic construct
GYSITSGYS

SEQ ID NO: 2 - protein, 8 aa, synthetic construct
GFTFTGYY

SEQ ID NO: 3 - protein, 8 aa, synthetic construct
GYTFTEYA

SEQ ID NO: 4 - protein, 8 aa, synthetic construct
GYTFTDYN

SEQ ID NO: 5 - protein, 8 aa, synthetic construct
GFTFSTYG

SEQ ID NO: 6 - protein, 8 aa, synthetic construct
GFTFNNYG

SEQ ID NO: 7 - protein, 9 aa, synthetic construct
GYSITSGFY

SEQ ID NO: 8 - protein, 9 aa, synthetic construct
GYSITSDYA

SEQ ID NO: 9 - protein, 8 aa, synthetic construct
GYTFTDYS

SEQ ID NO: 10 - protein, 8 aa, synthetic construct
GYTFTSYD

SEQ ID NO: 11 - protein, 7 aa, synthetic construct
MHYSATP

SEQ ID NO: 12 - protein, 7 aa, synthetic construct
IHYSTTT

SEQ ID NO: 13 - protein, 8 aa, synthetic construct
IYPGNVNT

SEQ ID NO: 14 - protein, 8 aa, synthetic construct
ITAYSGDA

SEQ ID NO: 15 - protein, 8 aa, synthetic construct
INPNNGGT

SEQ ID NO: 16 - protein, 8 aa, synthetic construct
INPNNGAT

SEQ ID NO: 17 - protein, 8 aa, synthetic construct
INSRASYT

SEQ ID NO: 18 - protein, 8 aa, synthetic construct
INRGGSYT

SEQ ID NO: 19 - protein, 7 aa, synthetic construct
INFGGTN

SEQ ID NO: 20 - protein, 7 aa, synthetic construct
ITYSGST

SEQ ID NO: 21 - protein, 8 aa, synthetic construct
INTETGEP

SEQ ID NO: 22 - protein, 8 aa, synthetic construct
IFPGDGST

SEQ ID NO: 23 - protein, 11 aa, synthetic construct
SRSLNWDVLDY

SEQ ID NO: 24 - protein, 14 aa, synthetic construct
ARSGYFASSYEFAY

SEQ ID NO: 25 - protein, 13 aa, synthetic construct
ARGLGRPNYGMDY

SEQ ID NO: 26 - protein, 13 aa, synthetic construct
ARSGYDDYYAFDY

SEQ ID NO: 27 - protein, 13 aa, synthetic construct
ARSGYDNYYALDY

SEQ ID NO: 28 - protein, 13 aa, synthetic construct
TRSGYDDYYALDY

SEQ ID NO: 29 - protein, 15 aa, synthetic construct
ARHRRDWDSYYAMDY

SEQ ID NO: 30 - protein, 11 aa, synthetic construct
ARGGNDGGFTY

SEQ ID NO: 31 - protein, 15 aa, synthetic construct
ARRIRRRTGDWYFDV

SEQ ID NO: 32 - protein, 10 aa, synthetic construct
VSYDYDAVSY

SEQ ID NO: 33 - protein, 8 aa, synthetic construct
ARYHFFDN

SEQ ID NO: 34 - protein, 10 aa, synthetic construct
KSVRTSGYNY

SEQ ID NO: 35 - protein, 10 aa, synthetic construct
KSVRTSGYSY

SEQ ID NO: 36 - protein, 6 aa, synthetic construct
ENIYSY

SEQ ID NO: 37 - protein, 12 aa, synthetic construct
QSLLNSRYQKNY

SEQ ID NO: 38 - protein, 5 aa, synthetic construct
SSVGY

SEQ ID NO: 39 - protein, 5 aa, synthetic construct
STVGY

SEQ ID NO: 40 - protein, 12 aa, synthetic construct
QSVLYNSNQKNY

SEQ ID NO: 41 - protein, 12 aa, synthetic construct
RSVLYNSNQKNY

SEQ ID NO: 42 - protein, 7 aa, synthetic construct
SSVSSTY

SEQ ID NO: 43 - protein, 12 aa, synthetic construct
QNLLNSVNQKNF

SEQ ID NO: 44 - protein, 6 aa, synthetic construct
QYVGAA

SEQ ID NO: 45 - protein, 11 aa, synthetic construct
KSLLYKDGKTY

SEQ ID NO: 46 - protein, 9 aa, synthetic construct
QHSREFPFT

SEQ ID NO: 47 - protein, 9 aa, synthetic construct
QHTREFPFT

SEQ ID NO: 48 - protein, 9 aa, synthetic construct
QHHYGTPLT

SEQ ID NO: 49 - protein, 9 aa, synthetic construct
QQHYGTPFT

SEQ ID NO: 50 - protein, 9 aa, synthetic construct
HQWSSYPST

SEQ ID NO: 51 - protein, 9 aa, synthetic construct
HQWSSYPCT

SEQ ID NO: 52 - protein, 9 aa, synthetic construct
HQWSNYPST

SEQ ID NO: 53 - protein, 8 aa, synthetic construct
HQYLSSWT

SEQ ID NO: 54 - protein, 9 aa, synthetic construct
HQWSGYPYT

SEQ ID NO: 55 - protein, 9 aa, synthetic construct
QNDYIYPYT

SEQ ID NO: 56 - protein, 8 aa, synthetic construct
QQYGNYRT

SEQ ID NO: 57 - protein, 9 aa, synthetic construct
QQLVEYPYT

SEQ ID NO: 58 - protein, 118 aa, synthetic construct
DVQLQESGPDLVKPYQSLSLTCTVTGYSITSGYSWHWIRQFPGNKLEWLGYMHYSATPNYNPSLKSRISITRDTSKNQFFLQLNSVTTEDTATYYCSRSLNWDVLDYWGQGTTLIVSS

SEQ ID NO: 59 - protein, 111 aa, synthetic construct
DIVLTQSPAYLPVSLGQRATISCRASKSVRTSGYNYLHWYQQKPGQPPKLLIYLASNPESGVPGRFSGSGSGTDFTLNIHPVEEEDSATYYCQHSREFPFTFGGGTQLEIK

SEQ ID NO: 60 - protein, 118 aa, synthetic construct
DVQLQESGPDLVKPSQSLSLTCTVTGYSITSGYSWHWIRQFPGNKLEWMGYIHYSTTTNYNPSLKSRISITRDTSKNQFFLQLSSVTTEDTATYFCSRSLNWDVLDYWGQGTTLIVSS

SEQ ID NO: 61 - protein, 111 aa, synthetic construct
DIVLTQSPAYLPVSLGQRATISCRASKSVRTSGYSYLHWYQQKPGQPPKLLIYLASNLESGVPDRFSGSGSGTDFTLNIHPVEEEDAATYYCQHTREFPFTFGGGTKLEIK

SEQ ID NO: 62 - protein, 121 aa, synthetic construct
QVQLQQSGPELVKPGTSVKISCKASGFTFTGYYLHWLKQRPGQGLEWIGWIYPGNVNTEFNEKFKGQATLTADKSSSTAYMQLSSLTSEDSAVYFCARSGYFASSYEFAYWGQGTLVTVSA

SEQ ID NO: 63 - protein, 107 aa, synthetic construct
DIQMTQSPASLSASVGETVTITCRASENIYSYLAWYQQKQGKSPQLLVYNAKTLAEGVPSRFSVSGSGTHFSLKINSLQPEDFGSYYCQHHYGTPLTFGAGTKLELK

SEQ ID NO: 64 - protein, 120 aa, synthetic construct
QVQLQQSGAELLRPGVSVKISCKGSGYTFTEYAMHWVKQSHAKSLEWIGVITAYSGDAFYNLKFKGKATVTVDKSSSTAYMELARLTSEDSAIYYCARGLGRPNYGMDYWGQGTSVTVSS

SEQ ID NO: 65 - protein, 113 aa, synthetic construct
DIVMTQSPSSLAMSVGQKVTMSCKSSQSLLNSRYQKNYLAWYQQKPGQSPKLLLYFASTRESGVPDRFIGSGSGTDFTLTISSVQAEDLADYFCQQHYGTPFTFGSGTKLEIK

SEQ ID NO: 66 - protein, 120 aa, synthetic construct
EVLLQQSGPELVKPGASVKIPCKASGYTFTDYNIDWVKQSHGKSLEWIGDINPNNGGTLYNQKFKAKATMTVDKSSSTAYMELRSLTSDDTAVYYCARSGYDDYYAFDYWGQGTSVTVSS

SEQ ID NO: 67 - protein, 106 aa, synthetic construct
QVVLTQSPAIMSAFLGEKITLTCSASSSVGYLHWYQQKSGTSPKLLIYSTSNVASGVPSRFSGSGSGTFYSLKISSVEAEDAADYYCHQWSSYPSTFGAGTRLELK

SEQ ID NO: 68 - protein, 120 aa, synthetic construct
EVLLQQSGPELVQPGTSVKIPCKASGYTFTDYNMDWVKQSHGKSLEWIGDINPNNGGTLYNQKFKGKATLSVDKSSSTAYMELRSLTSEDAAVYYCARSGYDNYYALDYWGQGTSVTVSS

SEQ ID NO: 69 - protein, 106 aa, synthetic construct
QIVLTQSPPIMSASLGEEITLTCSASSTVGYMHWFQQKSGTSPKLLIYSTSNLASGVPSRFSGSGSGTFYSLTISSVEAEDAADYYCHQWSSYPCTFGAGTKLELK

SEQ ID NO: 70 - protein, 120 aa, synthetic construct
EVLLQQSGPELVKPGASVKIPCKASGYTFTDYNMDWVKQSHGKSLEWIGDINPNNGATLYNQNFRGKATLTVDKSSSTAYMELRSLTSEDTAVYYCTRSGYDDYYALDYWGQGTSVTVSS

SEQ ID NO: 71 - protein, 106 aa, synthetic construct
QTVLTQSPAIMSASLGEEITLTCSASSSVGYMHWYQQKSGTSPKLLIYSTSNLASGVPSRFSGRGSGTFYSLTISTVEAEDAADYYCHQWSNYPSTFGTGTKLELK

SEQ ID NO: 72 - protein, 122 aa, synthetic construct
EVQLVESGGDLVRPGGSLKLSCAASGFTFSTYGMSWVRQSPDKRLEWVATINSRASYTYYPDSVKGRFTISRDNAKNTLYLQMSSLKSEDTGLYFCARHRRDWDSYYAMDYWGQGTSVTVSS

SEQ ID NO: 73 - protein, 112 aa, synthetic construct
NIMLTQSPSSLAVSTGDKVTMNCKSSQSVLYNSNQKNYLAWYQQKPGQSPTLLIYWASTSESGVPDRFTGSGSGTDFTLTISSVQAEDLAVYYCHQYLSSWTFGGGTKLEIK

SEQ ID NO: 74 - protein, 122 aa, synthetic construct
AVQLVESGGDLLRPGGSLKLSCVASGFTFNNYGMSWVRQTPDKRLEWVATINRGGSYTYYTDSVKGRFTISRDNAKKTLYLQMSSLKSEDTAMYYCARHRRDWDSYYAMDYWGQGTSVTVSS

SEQ ID NO: 75 - protein, 112 aa, synthetic construct
NIMMTQSPSSLAVSAGDKVAMSCKSSRSVLYNSNQKNYLAWYQLKPGQSPKLLIYWASTRESGVPDRFTGSGSGTDFTLTISSVQAEDLAVYYCHQYLSSWTFGGGTKVEIK

SEQ ID NO: 76 - protein, 118 aa, synthetic construct
DVQLQESGPGLVKPSQSLSLTCSVTGYSITSGFYWNWIRQLPGNKLEWLGYINFGGTNNYNPSLKIRGSITRDTSKNQFFLKLNSVTTEDTATYYCARGGNDGGFTYWGQGTLVTVSA

SEQ ID NO: 77 - protein, 108 aa, synthetic construct
ENVLTQSPAIVAASLGQKVTMTCRASSSVSSTYFHWYQQKSGASPKPLLHRTSNLASGVPARFSGSGSGTSYSLTISSVEAEDDATYYCHQWSGYPYTFGGGTKLEIK

SEQ ID NO: 78 - protein, 122 aa, synthetic construct
DVQLQESGPGLVKPSQSLSLTCTVTGYSITSDYAWHWIRQCPGNKLEWMAYITYSGSTSYNPSLKGRFSITRDTSKNQFFLQLNSVTSEDSATYYCARRIRRRTGDWYFDVWGAGTTVTVSS

SEQ ID NO: 79 - protein, 113 aa, synthetic construct
DIVMTQSPSSLTVTAGEKATMSCKSSQNLLNSVNQKNFLTWYQQKPGQPPKLLIYWASARESGVPDRFTGSGSGTDFTLTISSVQAEDLAVYYCQNDYIYPYTFGGGTKLEIK

SEQ ID NO: 80 - protein, 117 aa, synthetic construct
QIQLVQSGPELKKPGETVKISCKTSGYTFTDYSMHWVKQAPRKGLKWMGWINTETGEPTYADDFKGRFAFSLETSASTAYLQINNLRNEDTATYFCVSYDYDAVSYWGQGTLVTVSA

SEQ ID NO: 81 - protein, 106 aa, synthetic construct
DIVMTQSHKFMSTSVGDRVNITCKASQYVGAAVAWYQQKPGQSPKLLIYWASTRHTGVPDRFTGSGSGTDFTLTINNVQSEDLADYFCQQYGNYRTFGGGTKLEIK

SEQ ID NO: 82 - protein, 115 aa, synthetic construct
QVQLQQSGAELVKPGASVKLSCKASGYTFTSYDINWVRQRPEQGLEWIGWIFPGDGSTDYNEKFKGKATLTTDKSSSTAYMQLSRLTSEDSAVYFCARYHFFDNWGQGTTLTVSS

SEQ ID NO: 83 - protein, 112 aa, synthetic construct
DIVITQDELSNPVTSGESVSISCRSSKSLLYKDGKTYLSWFLQRPGQSPQLLIYLMSTRASGVSDRFSGSGSGTDFTLEISRVKAEDVGVYYCQQLVEYPYTFGGGTKLEIK

SEQ ID NO: 84 - protein, 120 aa, synthetic construct
QVQLVQSGAEVKKPGASVKVSCKASGYTFTDYNMDWVRQSPGQSLEWMGDINPNNGATLYNQNFRGRVTITRDTSASTAYMELSSLRSEDTAVYYCTRSGYDDYYALDYWGQGTTVTVSS

SEQ ID NO: 85 - protein, 106 aa, synthetic construct
DTQLTQSPSFLSASVGDRVTITCSASSSVGYMHWYQQKPGKAPKLLIYSTSNLASGVPSRFSGSGSGTEYTLTISSLQPEDFATYYCHQWSNYPSTFGQGTKLEIK

SEQ ID NO: 86 - protein, 115 aa, synthetic construct
QVQLVQSGAEVKKPGASVKVSCKASGYTFTSYDINWVRQAPGQGLEWMGWIFPGDGSTDYNEKFKGRVTMTTDKSSSTAYMELSRLRSDDTAVYYCARYHFFDNWGQGTTVTVSS

SEQ ID NO: 87 - protein, 112 aa, synthetic construct
DIVMTQSPLSLAVSLGERATINCRSSKSLLYKDGKTYLSWFQQKPGQPPKLLIYLMSTRASGVPDRFSGSGSGTDFTLTISSLQAEDVAVYYCQQLVEYPYTFGGGTKVEIK

SEQ ID NO: 88 - protein, 115 aa, synthetic construct
QVQLVQSGSELKKPGASVKVSCKASGYTFTSYDINWVRQAPGQGLEWMGWIFPGDGSTDYNEKFKGRAVLSTDTSVSTAYLQISSLKAEDTAVYYCARYHFFDNWGQGTTVTVSS

SEQ ID NO: 89 - protein, 112 aa, synthetic construct
DIVITQSPLSLAVSLGERATINCRSSKSLLYKDGKTYLSWFQQKPGQPPQLLIYLMSTRASGVPDRFSGSGSGTDFTLTISSLQAEDVAVYYCQQLVEYPYTFGGGTKVEIK

SEQ ID NO: 90 - protein, 15 aa, synthetic construct
QAGRRMDMDSSHSIT

SEQ ID NO: 91 - protein, 15 aa, synthetic construct
PISHPMGRGHQAGRR

SEQ ID NO: 92 - protein, 15 aa, synthetic construct
SHSITLQPTANPNTG

SEQ ID NO: 93 - protein, 8 aa, synthetic construct
GYTFTNYW

SEQ ID NO: 94 - protein, 8 aa, synthetic construct
IYPSDSYT

SEQ ID NO: 95 - protein, 8 aa, synthetic construct
TKWGDSDY

SEQ ID NO: 96 - protein, 6 aa, synthetic construct
QDINRY

SEQ ID NO: 97 - protein, 9 aa, synthetic construct
LQYDEFPFT

SEQ ID NO: 98 - protein, 115 aa, synthetic construct
QVHLQQPGAELVRPGASVKLSCKASGYTFTNYWINWVKQRPGQGLEWIGNIYPSDSYTNYNQKFKDKATLTVDKSSSTAYVHLSSPTSEDSAVYYCTKWGDSDYWGQGTTLTVSS

SEQ ID NO: 99 - protein, 107 aa, synthetic construct
DIKMTQSPSSMYASLGERVTITCKASQDINRYLRWFQQKPGKSPKTLIYRANRLVDGVPSRFSGSGSGQDYSLTISSLEYEDMGIYYCLQYDEFPFTFGSGTKLEIK

SEQ ID NO: 100 - protein, 115 aa, synthetic construct
QVQLVQSGSELKKPGASVKVSCKASGYTFTNYWINWVRQAPGQGLEWMGNIYPSDSYTNYNQKFKDRAVFSVDKSVSTAYLQISSLKAEDTAVYYCTKWGDSDYWGQGTTVTVSS

SEQ ID NO: 101 - protein, 107 aa, synthetic construct
DIQMTQSPSSLSASVGDRVTITCKASQDINRYLRWFQQKPGKSPKTLIYRANRLVDGVPSRFSGSGSGQDYTLTISSLQPEDFATYYCLQYDEFPFTFGQGTKLEIK

SEQ ID NO: 102 - protein, 330 aa, synthetic construct [sequence omitted]

SEQ ID NO: 103 - protein, 107 aa, synthetic construct [sequence omitted]

SEQ ID NO: 104 - protein, 107 aa, synthetic construct
DIQMTQSPSSLSASVGDRVTITCKASQDINRYLRWFQQKPGKSPKTLIYRANRLVDGVPSRFSGSGSGTDFTLTISSLQPEDFATYYCLQYDEFPFTFGQGTKLEIK